(19) United States
(12) Patent Application Publication
(10) Pub. No.: US 2025/0122142 A1
(43) Pub. Date: Apr. 17, 2025

(54) COMPOUND FOR ORGANIC ELECTRIC ELEMENT, ORGANIC ELECTRIC ELEMENT USING SAME, AND ELECTRONIC DEVICE THEREOF

(71) Applicant: DUK SAN NEOLUX CO., LTD., Cheonan-si, Chungcheongnam-do (KR)

(72) Inventors: Hyoung Keun PARK, Cheonan-si, Chungcheongnam-do (KR); Jung Geun LEE, Cheonan-si, Chungcheongnam-do (KR); Sun Hee LEE, Cheonan-si, Chungcheongnam-do (KR); Yong Wook PARK, Cheonan-si, Chungcheongnam-do (KR); Young Hoon KANG, Cheonan-si, Chungcheongnam-do (KR)

(73) Assignee: DUK SAN NEOLUX CO., LTD., Cheonan-si, Chungcheongnam-do (KR)

(21) Appl. No.: 18/834,015

(22) PCT Filed: Aug. 3, 2023
(86) PCT No.: PCT/KR2023/011431

§ 371 Date: Jul. 29, 2024

(30) Foreign Application Priority Data
Aug. 16, 2022 (KR) 10-2022-0101878

(51) Int. Cl. C07C 211/61; C07B 59/00; C07D 209/90; C07D 213/16; C07D 263/56; C07D 307/91; C07D 333/76; H10K 50/13; H10K 50/15; H10K 85/60

(52) U.S. Cl. CPC C07C 211/61; C07B 59/004; C07D 209/90; C07D 213/16; C07D 263/56; C07D 307/91; C07D 333/76; H10K 50/13; H10K 50/15; H10K 85/615; H10K 85/631; H10K 85/654; H10K 85/6572; H10K 85/6574; H10K 85/6576; C07B 2200/05

(57) ABSTRACT

Provided are a compound of Formula 1 that can improve the luminous efficiency, stability, and lifespan of an organic electronic element employing the compound, and the organic electronic element, and an electronic device thereof.

### BACKGROUND

### Technical Field

The present invention relates to compounds for organic electronic element, organic electronic elements using the same, and electronic devices thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, for example, may include a hole injection layer, a hole transport layer, an emitting layer, an electron transport layer, an electron injection layer etc.

A material used as an organic material layer in an organic electronic element may be classified into a light emitting material and a charge transport material, such as a hole injection material, a hole transport material, an electron transport material, an electron injection material etc. depending on its function.

Currently, the portable display market is increasing in size toward large-area displays, which requires greater power consumption than that required by existing portable displays. Therefore, power consumption has become a very important factor for portable displays that have a limited power source such as batteries, and issues of efficiency and lifespan must also be resolved.

Efficiency, lifespan and driving voltage are related to each other, and when the efficiency is increased, the driving voltage is relatively decreased, and as the driving voltage is decreased, crystallization of organic materials due to Joule heating generated during driving decreases, and consequently, the lifespan tends to increase. However, the efficiency cannot be maximized simply by improving the organic material layer. This is because, when the energy level and T1 value between each organic material layer, and the intrinsic properties (mobility, interfacial properties, etc.) of materials are optimally combined, long lifespan and high efficiency can be achieved at the same time.

That is, in order to sufficiently exhibit the excellent characteristics of the organic electronic element, a material for forming an organic material layer in an element such as a hole injection material, a hole transport material, a light emitting material, an electron transport material, an electron injection material, an emitting-auxiliary layer material should be supported by stable and efficient materials. However, such a stable and efficient organic material layer material for an organic electronic element has not been sufficiently developed yet. Therefore, development of new materials is continuously required.

### DETAILED DESCRIPTION OF THE INVENTION

### Summary

In order to solve the problems of the above-mentioned background technology, the present invention has discovered a compound with a novel structure, and has revealed the fact that when the compound is applied to an organic electronic element, the luminous efficiency, stability, and lifespan of the element can be greatly improved. Accordingly, the purpose of the present invention is to provide a novel compound, an organic electronic element using the same, and an electronic device thereof.

### Technical Solution

The present invention provides a compound represented by Formula 1.

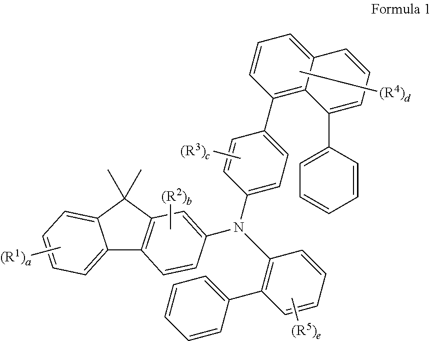

In another aspect, the present invention provides an organic electronic element and an electronic device comprising the compound represented by Formula 1.

### Effects of the Invention

By using the compound according to the present invention, it is possible to achieve a high luminous efficiency, a low driving voltage, and a high heat resistance of the element, and can greatly improve the color purity and lifespan of the element.

### BRIEF DESCRIPTION OF THE DRAWINGS

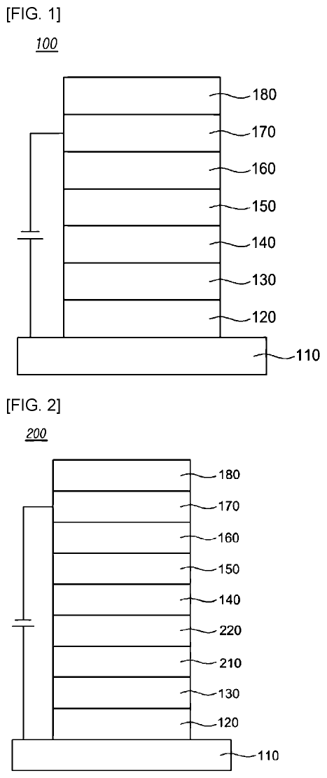

FIG. 1 to FIG. 3 illustrate an example of an organic electronic element
according to the present invention.

| | |
|---|---|
| 100, 200, 300: organic electronic | 110: the first electrode |
| element | |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected “,” coupled” or “connected” between each component.

As used in the specification and the accompanying claims, unless otherwise stated, the following is the meaning of the term as follows.

Unless otherwise stated, the term “halo” or “halogen”, as used herein, includes fluorine (F), bromine (Br), chlorine (Cl), or iodine (I).

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

Unless otherwise stated, the term “aryl group” or “arylene group”, as used herein, has 6 to 60 carbon atoms, but is not limited thereto. Herein, the aryl group or arylene group means a monocyclic and polycyclic aromatic group, and may also be formed in conjunction with an adjacent group. Examples of “aryl group” may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of monocyclic and polycyclic rings, and may include heteroaliphatic ring and/or heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes compound below.

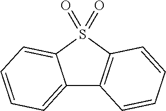 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

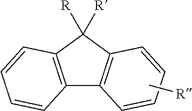

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro-’ and ‘tri-spiro-’, respectively, depending on the number of atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include one or more heteroatoms, but are not limited thereto.

Unless otherwise stated, the term “substituted or unsubstituted”, as used herein, means that substitution is substituted by at least one substituent selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiophen group, a C₆-C₂₀ arylthiophen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited thereto.

Unless otherwise expressly stated, the Formula used in the present invention, as used herein, is applied in the same manner as the substituent definition according to the definition of the exponent of the following Formula.

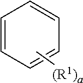

wherein, when a is an integer of 0, the substituent R¹ is absent, when a is an integer of 1, the sole substituent R¹ is linked to any one of the carbon constituting the benzene ring, when a is an integer of 2 or 3, each substituent R¹s may be the same and different, when a is an integer of 4 to 6, and is linked to the benzene ring in a similar manner, whereas the indication of hydrogen bound to the carbon forming the benzene ring is omitted.

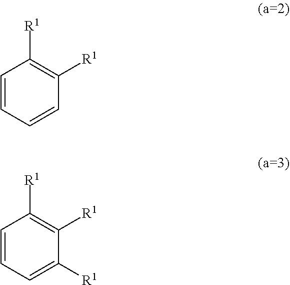

Hereinafter, a compound according to one aspect of the present invention and an organic electronic element comprising the same will be described.

The present invention provides a compound represented by Formula 1.

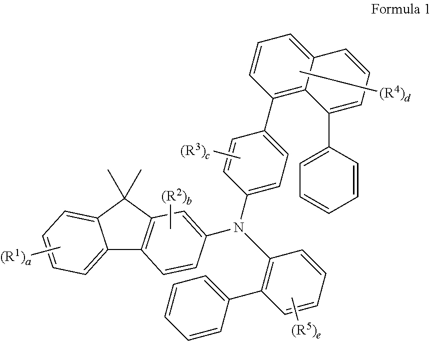

Wherein:
1) R¹, R², R³, R⁴ and R⁵ are each independently the same or different, and each independently selected from the group consisting of hydrogen; deuterium; cyano group: an C₆-C₆₀ aryl group; fluorenyl group; a C₂-C₆₀ heteroaryl group including at least one heteroatom of O, N, S, Si or P; a C₃-C₆₀ cycloalkyl group; a C₁-C₆₀ alkyl group; a C₂-C₂₀ alkenyl group; a C₂-C₂₀ alkynyl group; a C₁-C₃₀ alkoxyl group; and a C₆-C₃₀ aryloxy group; however, adjacent groups cannot be bonded to each other to form a ring,
Wherein when R¹, R², R³, R⁴ and R⁵ are an aryl group, preferably a C₆-C₃₀ aryl group, more preferably a C₆-C₂₅ aryl group, for example, it may be phenyl, biphenyl, naphthyl, terphenyl, phenanthrenyl, triphenylenyl, etc,
Wherein when R¹, R², R³, R⁴ and R⁵ are a heteroaryl group, preferably a C₂˜C₃₀ heteroaryl group, more preferably a C₂˜C₂₄ heteroaryl group, for example, pyridine, pyrimidine, pyrazine, quinoline, quinazoline, quinoxaline, dibenzofuran, dibenzothiophene, carbazole, naphthobenzofuran, naphthobenzothiophene, benzocarbazole, benzofuropyrimidine, benzothienopyrimidine, pyrimidoindole, 5-phenyl-5H-pyrimido[5,4-b]indole, benzoquinazoline, dibenzoquinazoline, phenothiazine, phenylphenothiazine, etc.,
Wherein when R¹, R², R³, R⁴ and R⁵ are cycloalkyl groups, preferably C₃-C₃₀ cycloalkyl groups; more preferably a C₃-C₂₀ cycloalkyl group.
Wherein when R¹, R², R³, R⁴ and R⁵ are an alkyl group, preferably a C₁-C₃₀ alkyl group, more preferably a C₁-C₂₀ alkyl group.
Wherein when R¹, R², R³, R⁴ and R⁵ are alkoxyl groups, preferably C₁˜C₂₀ alkoxyl groups.
Wherein when R¹, R², R³, R⁴ and R⁵ are an aryloxy group, preferably an C₆˜C₂₀ aryloxy group.
2) a, c and e are independently integers from 0 to 4, b is an integer from 0 to 3, d is an integer from 0 to 6,
3) wherein the aryl group, heteroaryl group, fluorenyl group, cycloalkyl group, alkyl group, alkenyl group, alkynyl group, alkoxyl group and aryloxy group may be substituted with one or more substituents selected from the group consisting of deuterium; cyano group; nitro group; C₁-C₂₀ alkylthio group; C₁-C₂₀ alkoxyl group; C₆-C₂₀ aryloxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and additionally, the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, any one of R¹, R², R³, R⁴ and R⁵ is selected from the group consisting of an C₁-C₁₀ alkyl group; C₃-C₁₀ cycloalkyl group; and an C₆-C₁₈ aryl group.

Also, any one of R¹, R², R³, R⁴ and R⁵ is selected from the group consisting of hydrogen; deuterium; and substituents represented by Formulas R-1 to R-13:

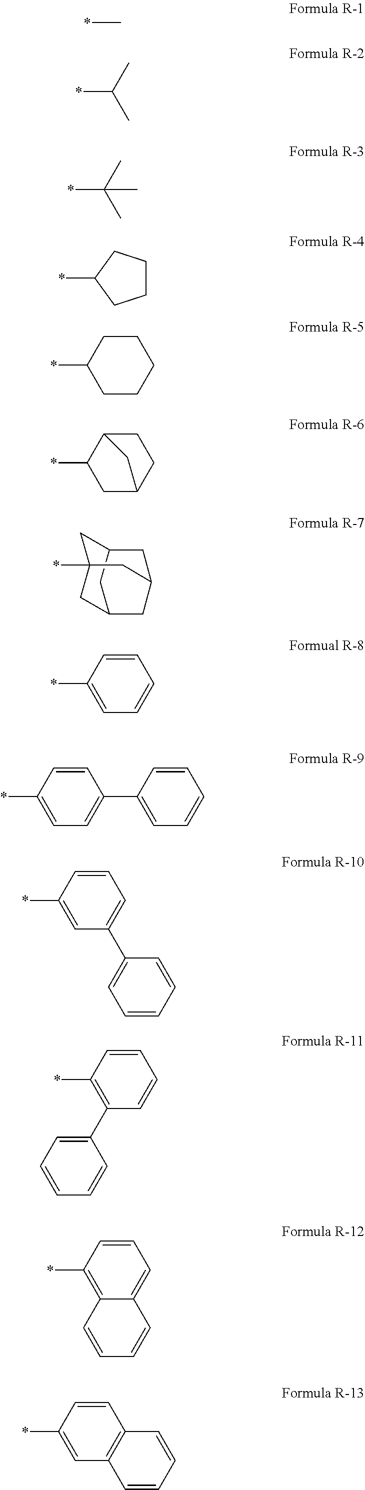

Wherein, * indicates the position to be bonded.

Also, R¹, R², R³, R⁴ and R⁵ are hydrogen; or deuterium;

Also, Formula 1 is represented by any of Formulas 1-1 to 1-9:

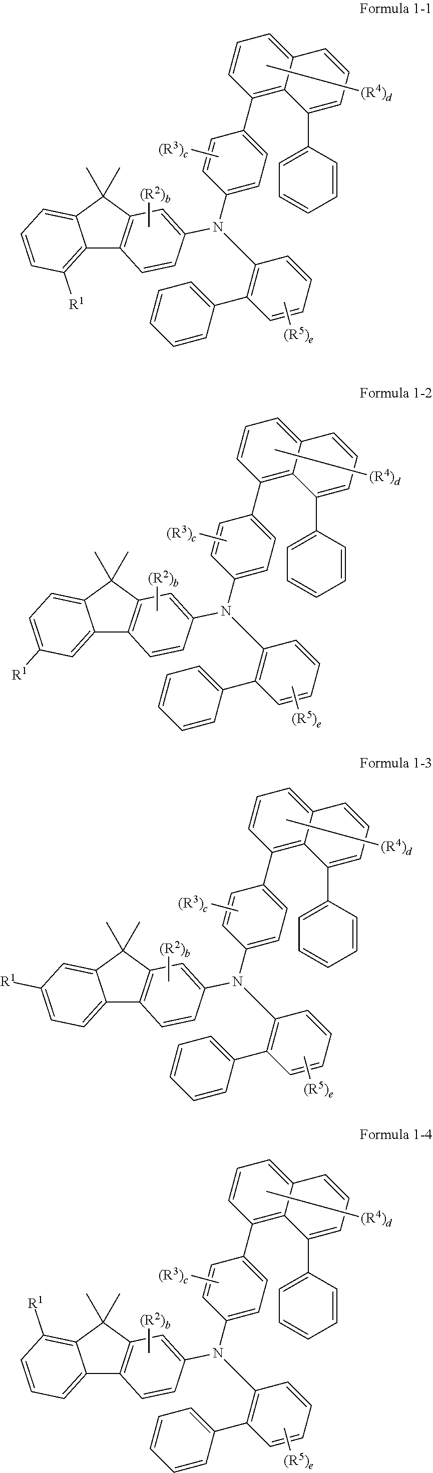

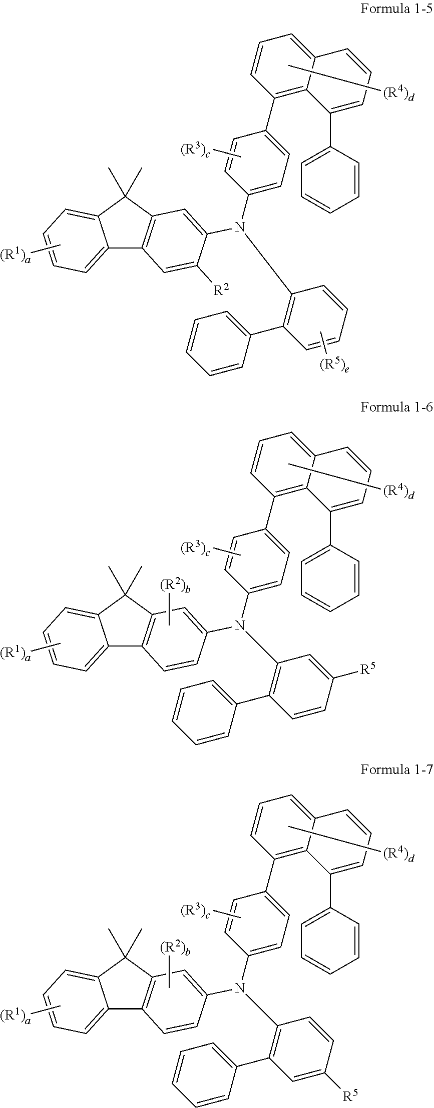

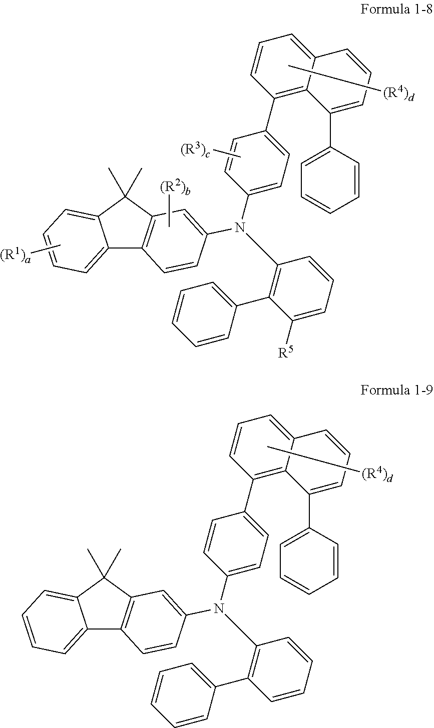

Wherein: R¹, R², R³, R⁴, R⁵, a, b, c, d and e are the same as defined above.

Also, the compound represented by Formula 1 is represented by any one of the following compounds P-1 to P-76.

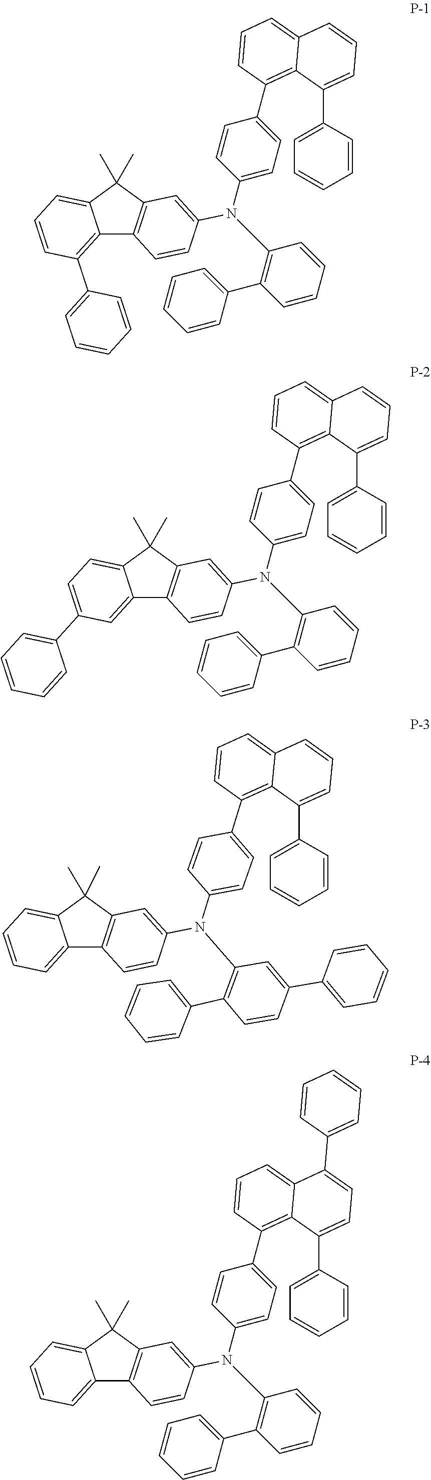

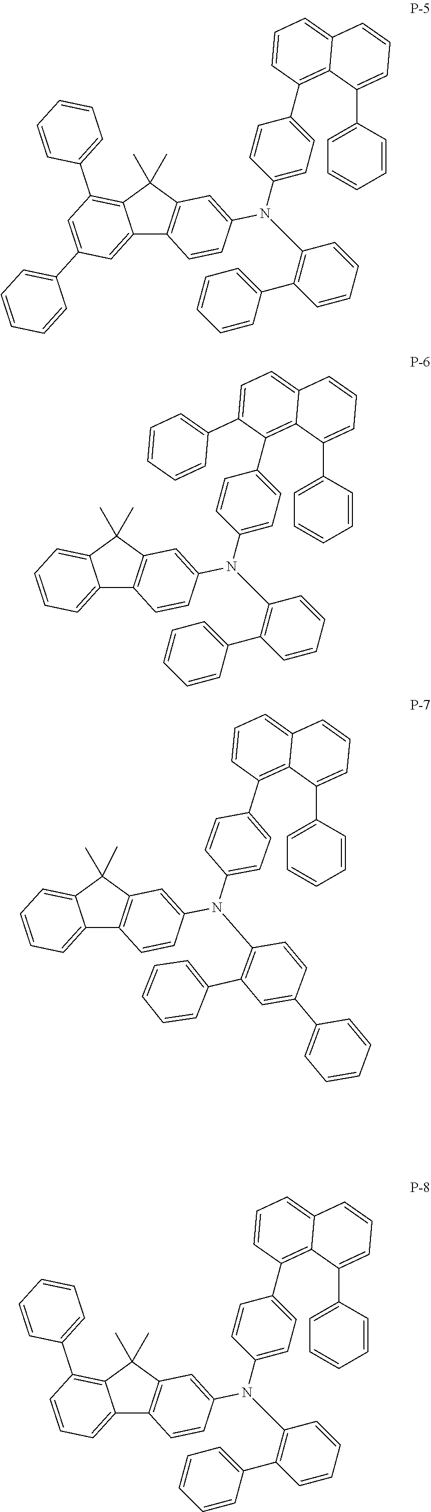 [SMILES: CC1(C)c2cc(N(c3ccc(-c4cccc5cccc(-c6ccccc6)c45)cc3)c3ccccc3-c3ccccc3)ccc2-c2cc(-c3ccccc3)cc(-c3ccccc3)c21 ; CC1(C)c2ccccc2-c2ccc(N(c3ccc(-c4c(-c5ccccc5)ccc5cccc(-c6ccccc6)c45)cc3)c3ccccc3-c3ccccc3)cc21 ; CC1(C)c2ccccc2-c2ccc(N(c3ccc(-c4cccc5cccc(-c6ccccc6)c45)cc3)c3ccc(-c4ccccc4)cc3-c3ccccc3)cc21 ; CC1(C)c2cc(N(c3ccc(-c4cccc5cccc(-c6ccccc6)c45)cc3)c3ccccc3-c3ccccc3)ccc2-c2cccc(-c3ccccc3)c21]

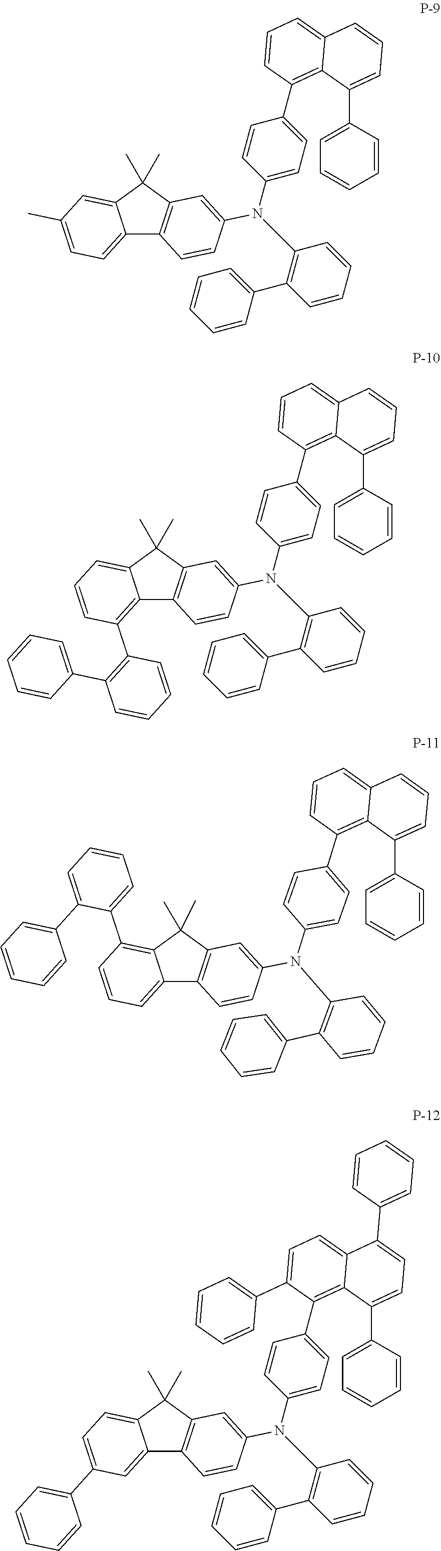 [SMILES: Cc1ccc2c(c1)C(C)(C)c1cc(N(c3ccc(-c4cccc5cccc(-c6ccccc6)c45)cc3)c3ccccc3-c3ccccc3)ccc1-2 ; CC1(C)c2cc(N(c3ccc(-c4cccc5cccc(-c6ccccc6)c45)cc3)c3ccccc3-c3ccccc3)ccc2-c2c(-c3ccccc3-c3ccccc3)cccc21 ; CC1(C)c2cc(N(c3ccc(-c4cccc5cccc(-c6ccccc6)c45)cc3)c3ccccc3-c3ccccc3)ccc2-c2cccc(-c3ccccc3-c3ccccc3)c21 ; CC1(C)c2ccc(-c3ccccc3)cc2-c2ccc(N(c3ccc(-c4c(-c5ccccc5)ccc5c(-c6ccccc6)ccc(-c6ccccc6)c45)cc3)c3ccccc3-c3ccccc3)cc21]

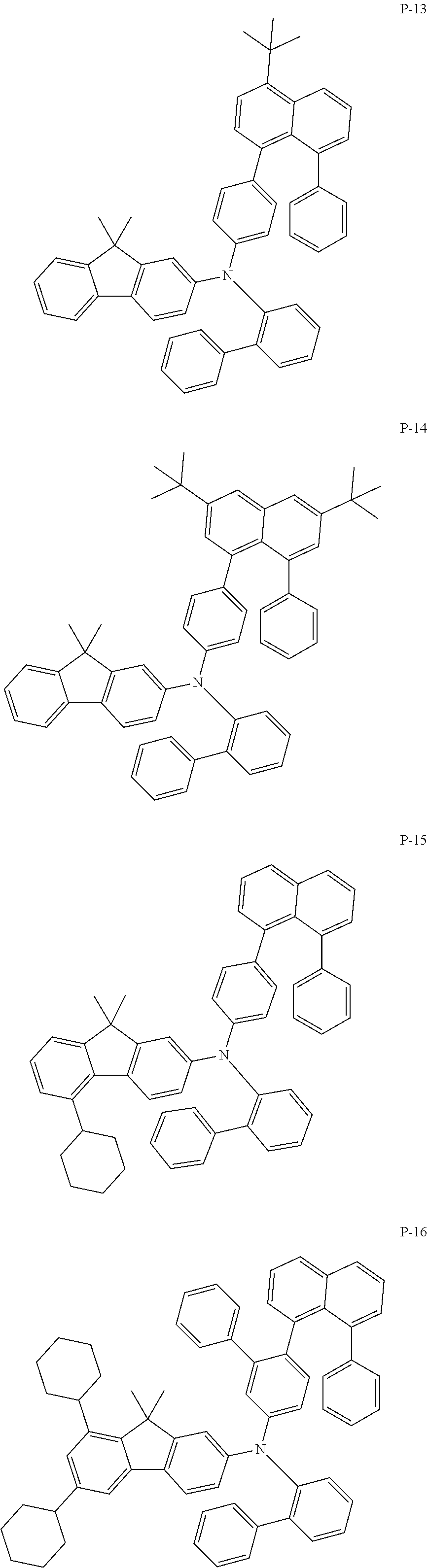 [SMILES: CC(C)(C)c1ccc(-c2ccc(N(c3ccc4c(c3)C(C)(C)c3ccccc3-4)c3ccccc3-c3ccccc3)cc2)c2c(-c3ccccc3)cccc12 ; CC(C)(C)c1cc(-c2ccccc2)c2c(-c3ccc(N(c4ccc5c(c4)C(C)(C)c4ccccc4-5)c4ccccc4-c4ccccc4)cc3)cc(C(C)(C)C)cc2c1 ; CC1(C)c2cc(N(c3ccc(-c4cccc5cccc(-c6ccccc6)c45)cc3)c3ccccc3-c3ccccc3)ccc2-c2c(C3CCCCC3)cccc21 ; CC1(C)c2cc(N(c3ccc(-c4cccc5cccc(-c6ccccc6)c45)c(-c4ccccc4)c3)c3ccccc3-c3ccccc3)ccc2-c2cc(C3CCCCC3)cc(C3CCCCC3)c21]

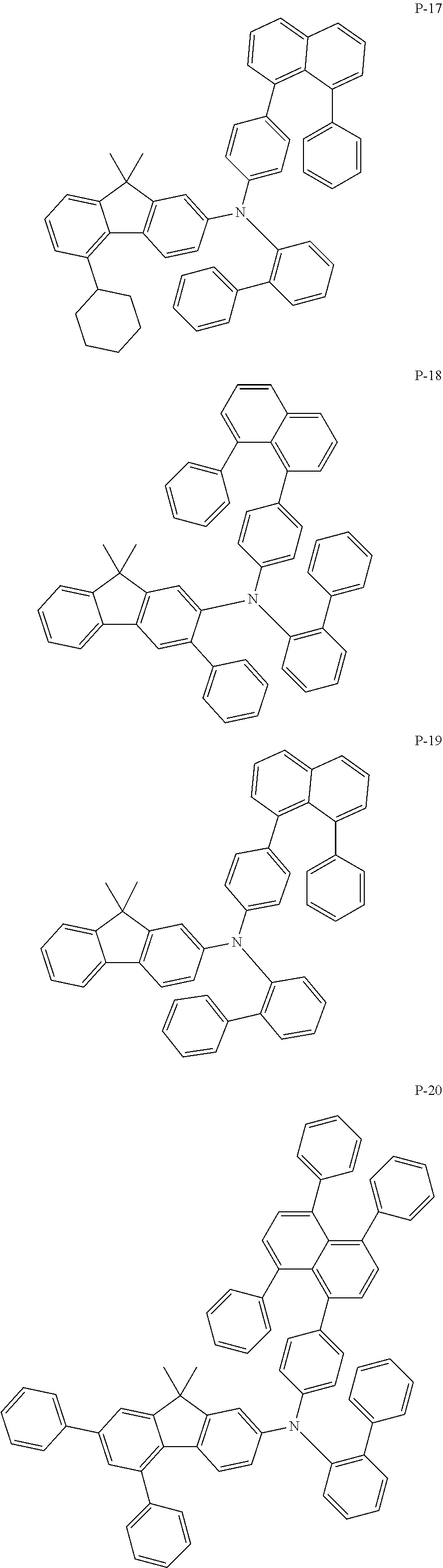

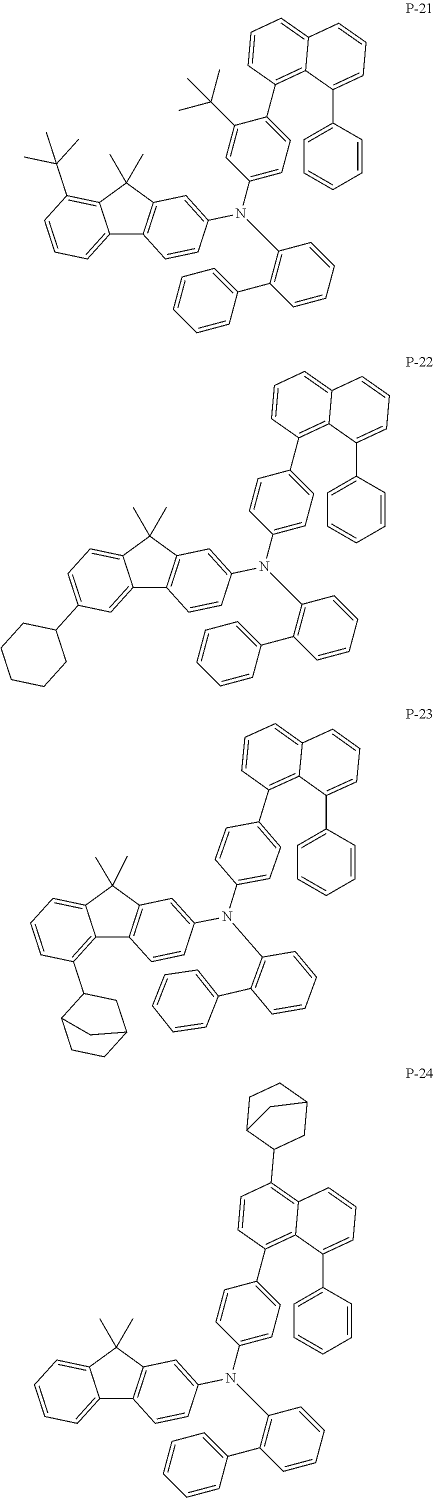 [SMILES: CC(C)(C)c1cc(N(c2ccc3c(c2)C(C)(C)c2c-3cccc2C(C)(C)C)c2ccccc2-c2ccccc2)ccc1-c1cccc2cccc(-c3ccccc3)c12 ; CC1(C)c2ccc(C3CCCCC3)cc2-c2ccc(N(c3ccc(-c4cccc5cccc(-c6ccccc6)c45)cc3)c3ccccc3-c3ccccc3)cc21 ; CC1(C)c2cc(N(c3ccc(-c4cccc5cccc(-c6ccccc6)c45)cc3)c3ccccc3-c3ccccc3)ccc2-c2c(C3CC4CCC3C4)cccc21 ; CC1(C)c2ccccc2-c2ccc(N(c3ccc(-c4ccc(C5CC6CCC5C6)c5cccc(-c6ccccc6)c45)cc3)c3ccccc3-c3ccccc3)cc21]

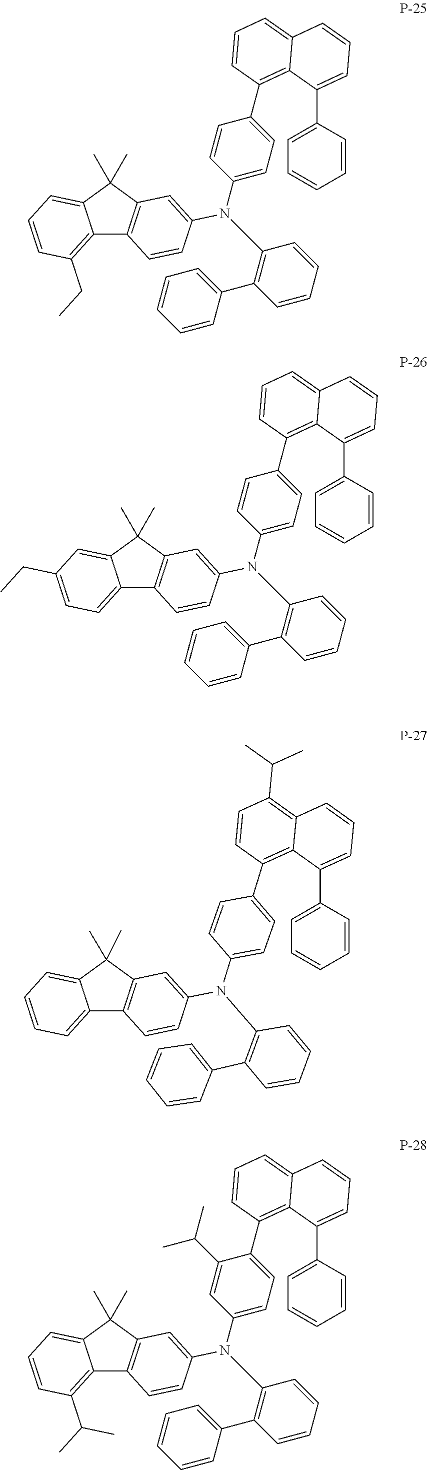 [SMILES: CCc1cccc2c1-c1ccc(N(c3ccc(-c4cccc5cccc(-c6ccccc6)c45)cc3)c3ccccc3-c3ccccc3)cc1C2(C)C ; CCc1ccc2c(c1)C(C)(C)c1cc(N(c3ccc(-c4cccc5cccc(-c6ccccc6)c45)cc3)c3ccccc3-c3ccccc3)ccc1-2 ; CC(C)c1ccc(-c2ccc(N(c3ccc4c(c3)C(C)(C)c3ccccc3-4)c3ccccc3-c3ccccc3)cc2)c2c(-c3ccccc3)cccc12 ; CC(C)c1cc(N(c2ccc3c(c2)C(C)(C)c2cccc(C(C)C)c2-3)c2ccccc2-c2ccccc2)ccc1-c1cccc2cccc(-c3ccccc3)c12]

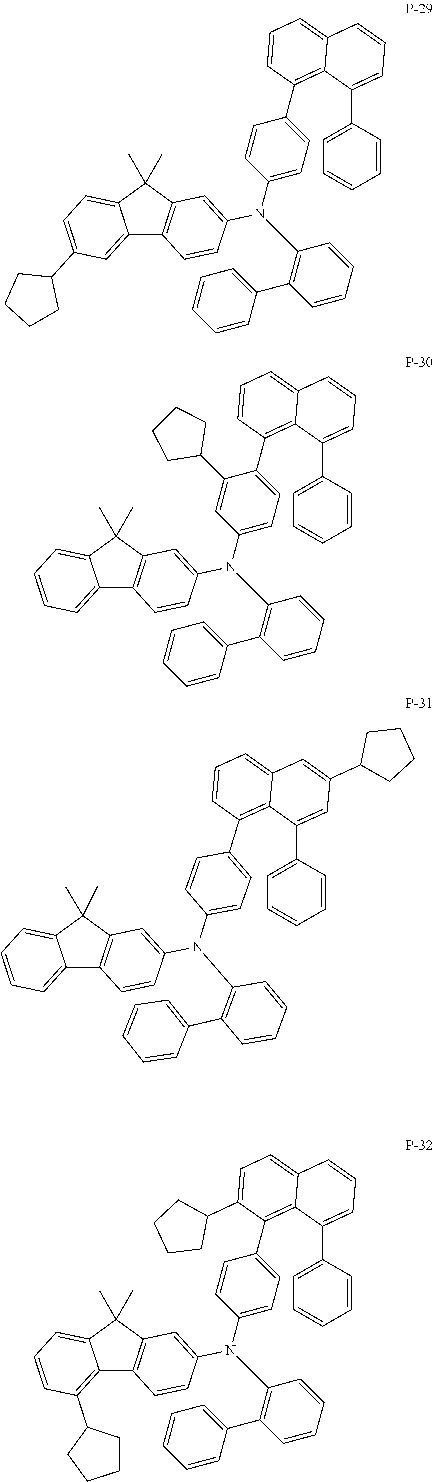 [SMILES: CC1(C)c2ccc(C3CCCC3)cc2-c2ccc(N(c3ccc(-c4cccc5cccc(-c6ccccc6)c45)cc3)c3ccccc3-c3ccccc3)cc21 ; CC1(C)c2ccccc2-c2ccc(N(c3ccc(-c4cccc5cccc(-c6ccccc6)c45)c(C4CCCC4)c3)c3ccccc3-c3ccccc3)cc21 ; CC1(C)c2ccccc2-c2ccc(N(c3ccc(-c4cccc5cc(C6CCCC6)cc(-c6ccccc6)c45)cc3)c3ccccc3-c3ccccc3)cc21 ; CC1(C)c2cc(N(c3ccc(-c4c(C5CCCC5)ccc5cccc(-c6ccccc6)c45)cc3)c3ccccc3-c3ccccc3)ccc2-c2c(C3CCCC3)cccc21]

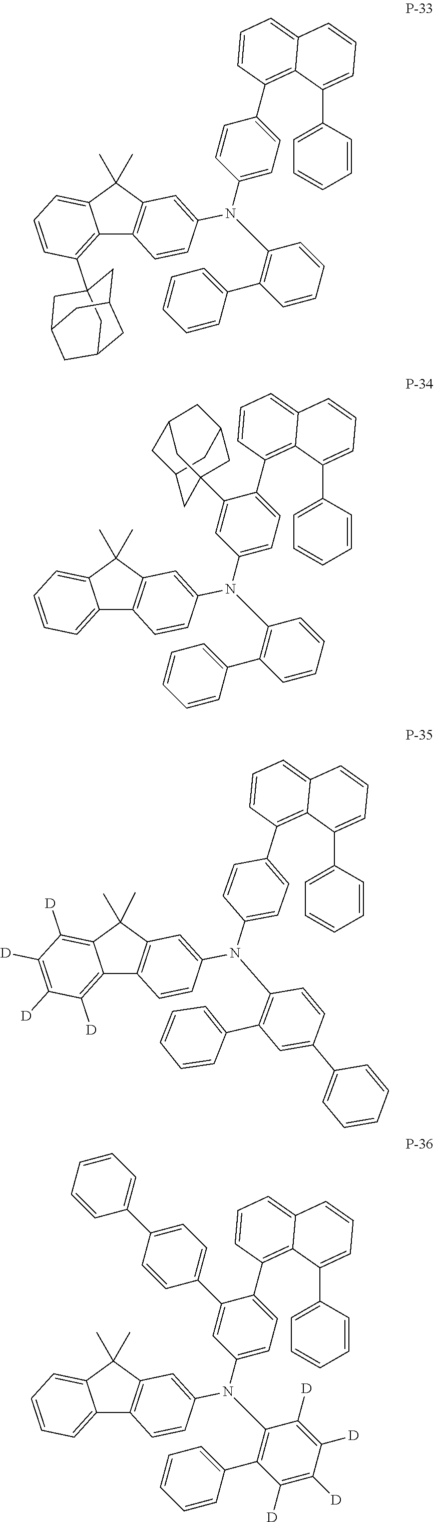 [SMILES: CC1(C)c2cc(N(c3ccc(-c4cccc5cccc(-c6ccccc6)c45)cc3)c3ccccc3-c3ccccc3)ccc2-c2c(C34CC5CC(CC(C5)C3)C4)cccc21 ; CC1(C)c2ccccc2-c2ccc(N(c3ccc(-c4cccc5cccc(-c6ccccc6)c45)c(C45CC6CC(CC(C6)C4)C5)c3)c3ccccc3-c3ccccc3)cc21 ; [2H]c1c([2H])c([2H])c2c(c1[2H])-c1ccc(N(c3ccc(-c4cccc5cccc(-c6ccccc6)c45)cc3)c3ccc(-c4ccccc4)cc3-c3ccccc3)cc1C2(C)C ; [2H]c1c([2H])c([2H])c(N(c2ccc(-c3cccc4cccc(-c5ccccc5)c34)c(-c3ccc(-c4ccccc4)cc3)c2)c2ccc3c(c2)C(C)(C)c2ccccc2-3)c(-c2ccccc2)c1[2H]]

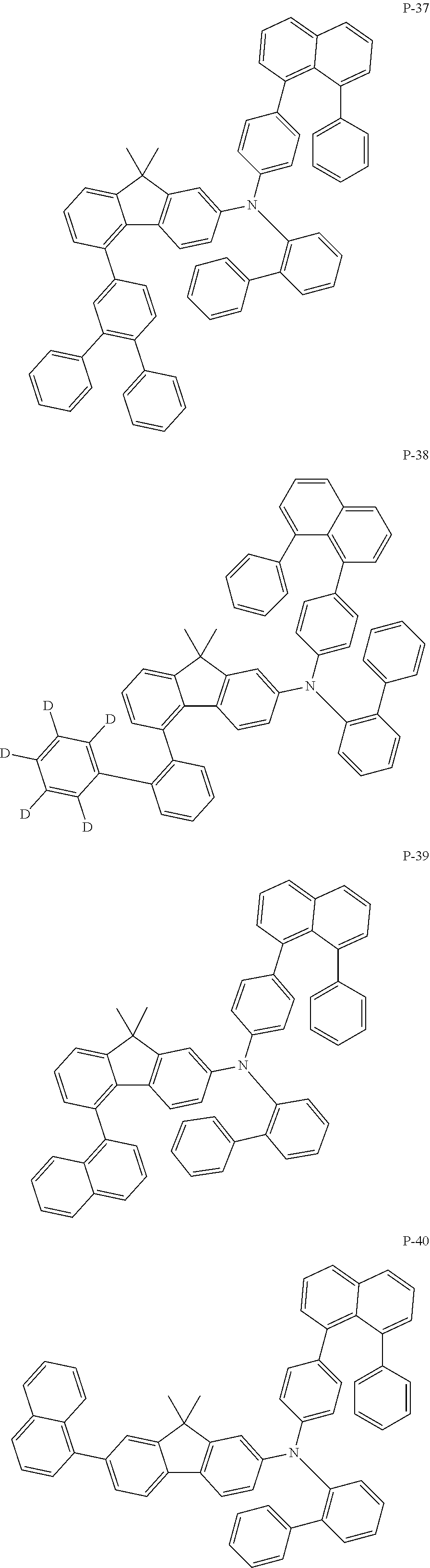 [SMILES: CC1(C)c2cc(N(c3ccc(-c4cccc5cccc(-c6ccccc6)c45)cc3)c3ccccc3-c3ccccc3)ccc2-c2c(-c3ccc(-c4ccccc4)c(-c4ccccc4)c3)cccc21 ; [2H]c1c([2H])c([2H])c(-c2ccccc2-c2cccc3c2-c2ccc(N(c4ccc(-c5cccc6cccc(-c7ccccc7)c56)cc4)c4ccccc4-c4ccccc4)cc2C3(C)C)c([2H])c1[2H] ; CC1(C)c2cc(N(c3ccc(-c4cccc5cccc(-c6ccccc6)c45)cc3)c3ccccc3-c3ccccc3)ccc2-c2c(-c3cccc4ccccc34)cccc21 ; CC1(C)c2cc(-c3cccc4ccccc34)ccc2-c2ccc(N(c3ccc(-c4cccc5cccc(-c6ccccc6)c45)cc3)c3ccccc3-c3ccccc3)cc21]

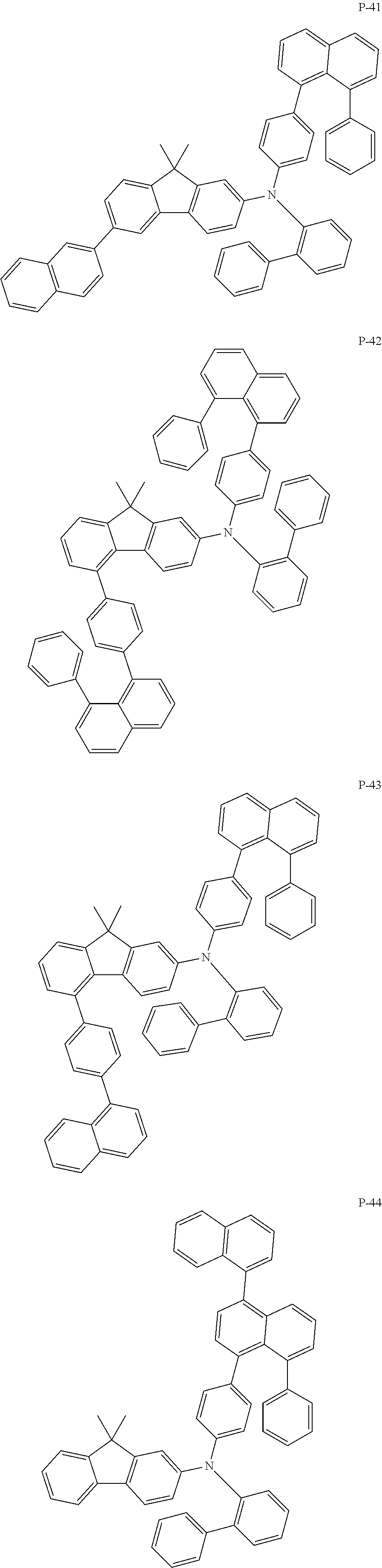 [SMILES: CC1(C)c2ccc(-c3ccc4ccccc4c3)cc2-c2ccc(N(c3ccc(-c4cccc5cccc(-c6ccccc6)c45)cc3)c3ccccc3-c3ccccc3)cc21 ; CC1(C)c2cc(N(c3ccc(-c4cccc5cccc(-c6ccccc6)c45)cc3)c3ccccc3-c3ccccc3)ccc2-c2c(-c3ccc(-c4cccc5cccc(-c6ccccc6)c45)cc3)cccc21 ; CC1(C)c2cc(N(c3ccc(-c4cccc5cccc(-c6ccccc6)c45)cc3)c3ccccc3-c3ccccc3)ccc2-c2c(-c3ccc(-c4cccc5ccccc45)cc3)cccc21 ; CC1(C)c2ccccc2-c2ccc(N(c3ccc(-c4ccc(-c5cccc6ccccc56)c5cccc(-c6ccccc6)c45)cc3)c3ccccc3-c3ccccc3)cc21]

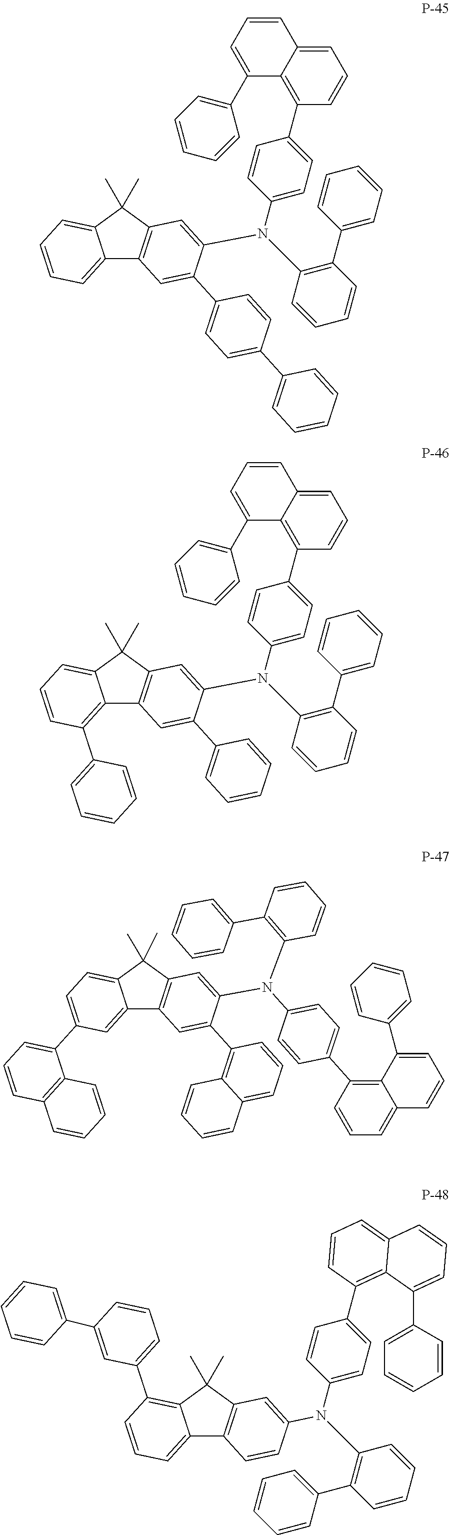

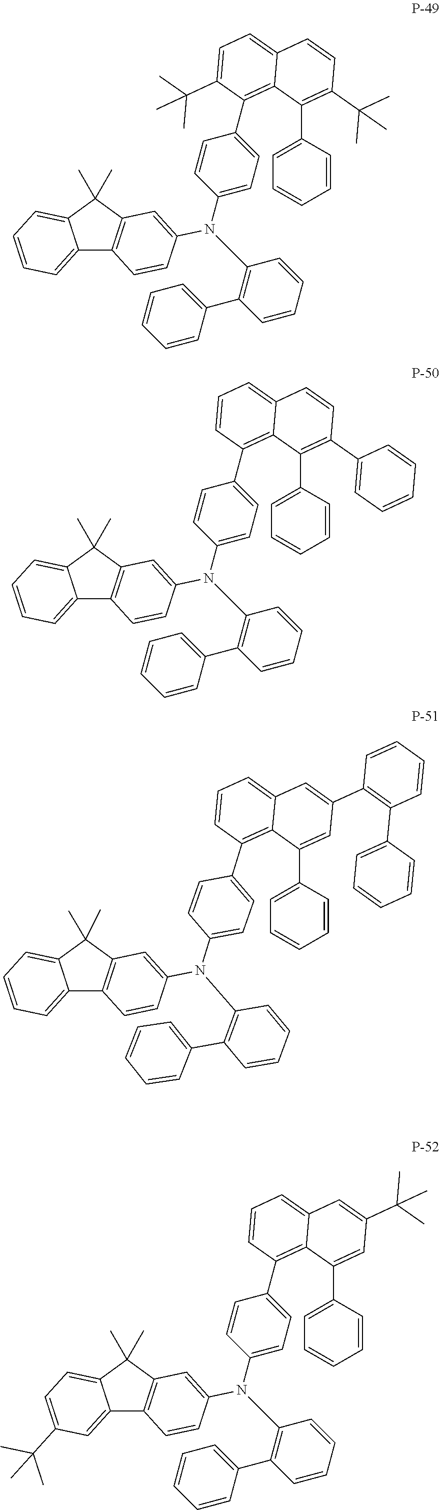

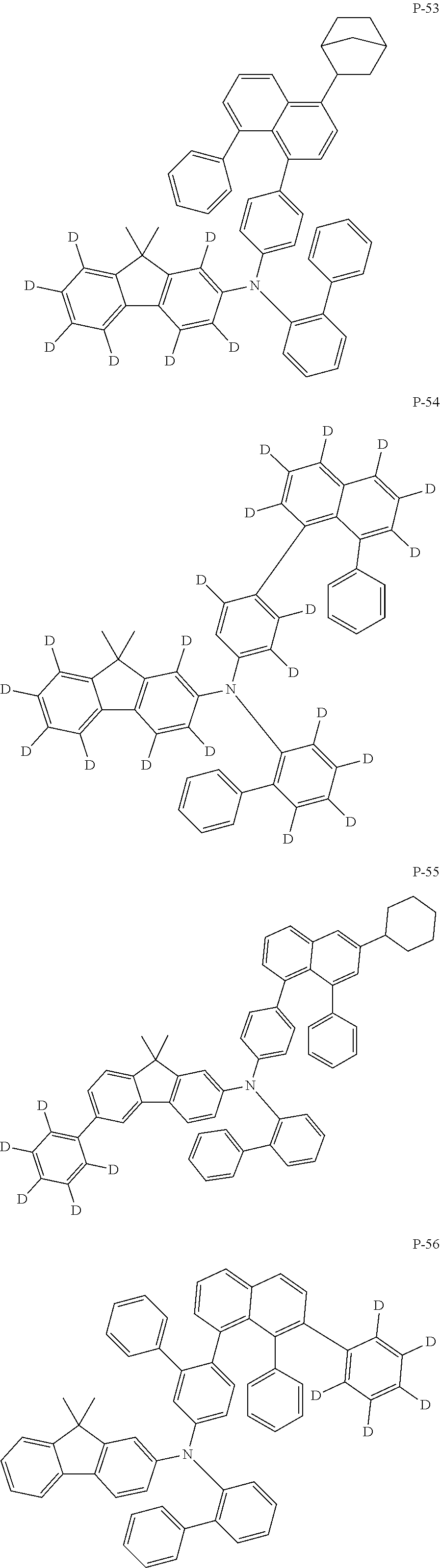

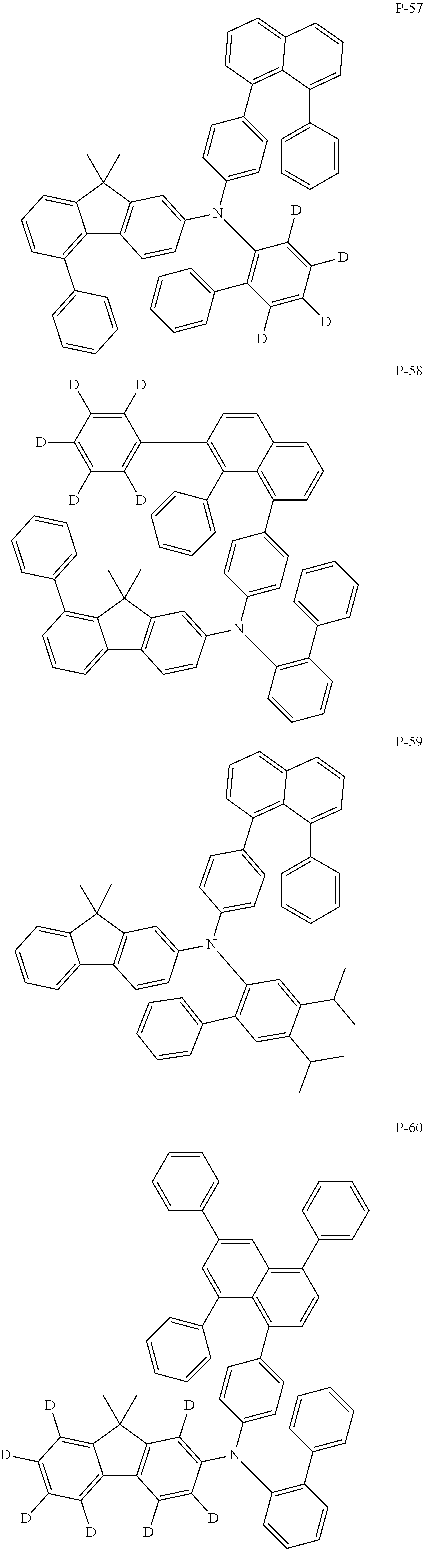

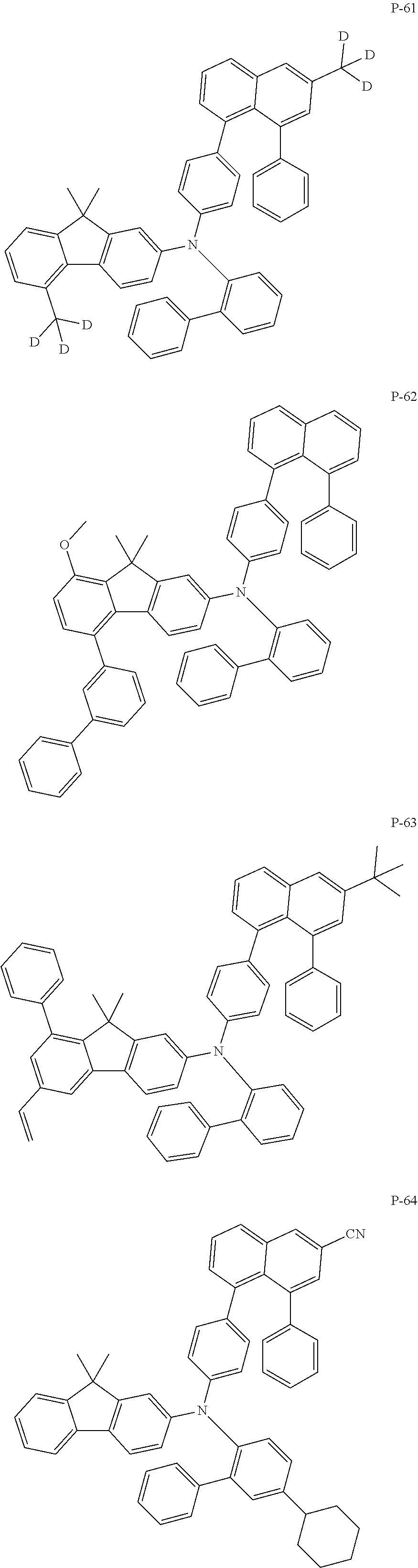 [SMILES: [2H]C([2H])([2H])c1cc(-c2ccccc2)c2c(-c3ccc(N(c4ccc5c(c4)C(C)(C)c4cccc(C([2H])([2H])[2H])c4-5)c4ccccc4-c4ccccc4)cc3)cccc2c1 ; COc1ccc(-c2cccc(-c3ccccc3)c2)c2c1C(C)(C)c1cc(N(c3ccc(-c4cccc5cccc(-c6ccccc6)c45)cc3)c3ccccc3-c3ccccc3)ccc1-2 ; C=Cc1cc(-c2ccccc2)c2c(c1)-c1ccc(N(c3ccc(-c4cccc5cc(C(C)(C)C)cc(-c6ccccc6)c45)cc3)c3ccccc3-c3ccccc3)cc1C2(C)C ; CC1(C)c2ccccc2-c2ccc(N(c3ccc(-c4cccc5cc(C#N)cc(-c6ccccc6)c45)cc3)c3ccc(C4CCCCC4)cc3-c3ccccc3)cc21]

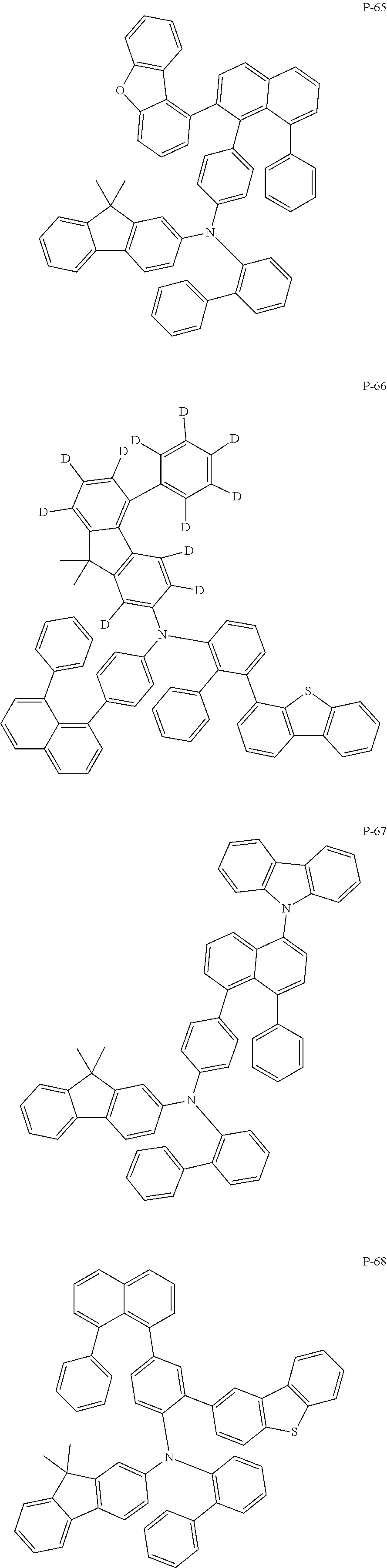 [SMILES: CC1(C)c2ccccc2-c2ccc(N(c3ccc(-c4c(-c5cccc6oc7ccccc7c56)ccc5cccc(-c6ccccc6)c45)cc3)c3ccccc3-c3ccccc3)cc21 ; [2H]c1c([2H])c([2H])c(-c2c([2H])c([2H])c([2H])c3c2-c2c([2H])c([2H])c(N(c4ccc(-c5cccc6cccc(-c7ccccc7)c56)cc4)c4cccc(-c5cccc6c5sc5ccccc56)c4-c4ccccc4)c([2H])c2C3(C)C)c([2H])c1[2H] ; CC1(C)c2ccccc2-c2ccc(N(c3ccc(-c4cccc5c(-n6c7ccccc7c7ccccc76)ccc(-c6ccccc6)c45)cc3)c3ccccc3-c3ccccc3)cc21 ; CC1(C)c2ccccc2-c2ccc(N(c3ccccc3-c3ccccc3)c3ccc(-c4cccc5cccc(-c6ccccc6)c45)cc3-c3ccc4sc5ccccc5c4c3)cc21]

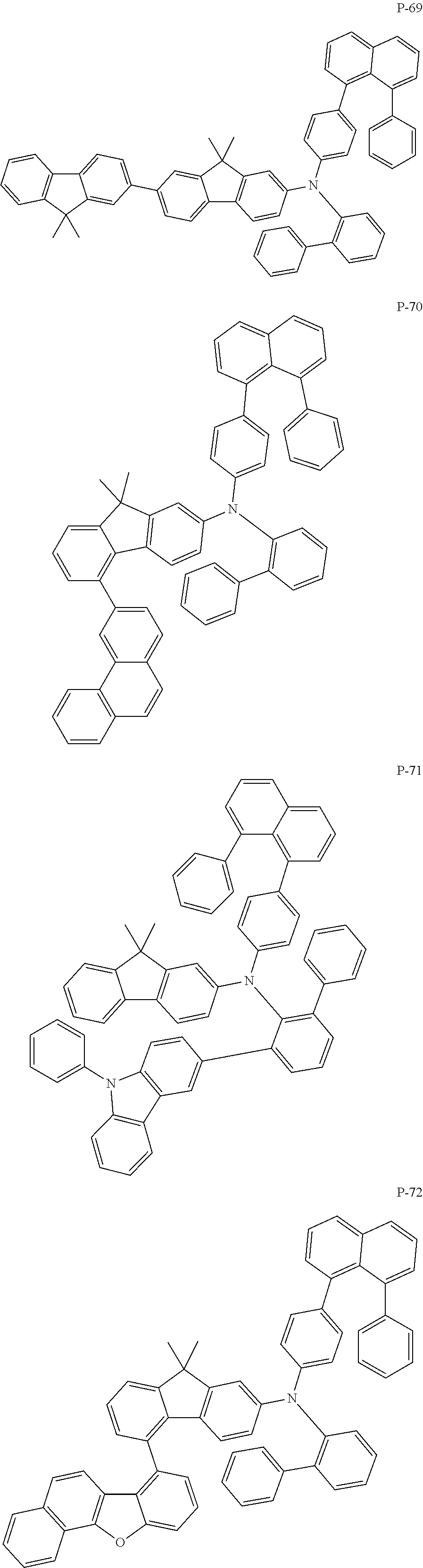

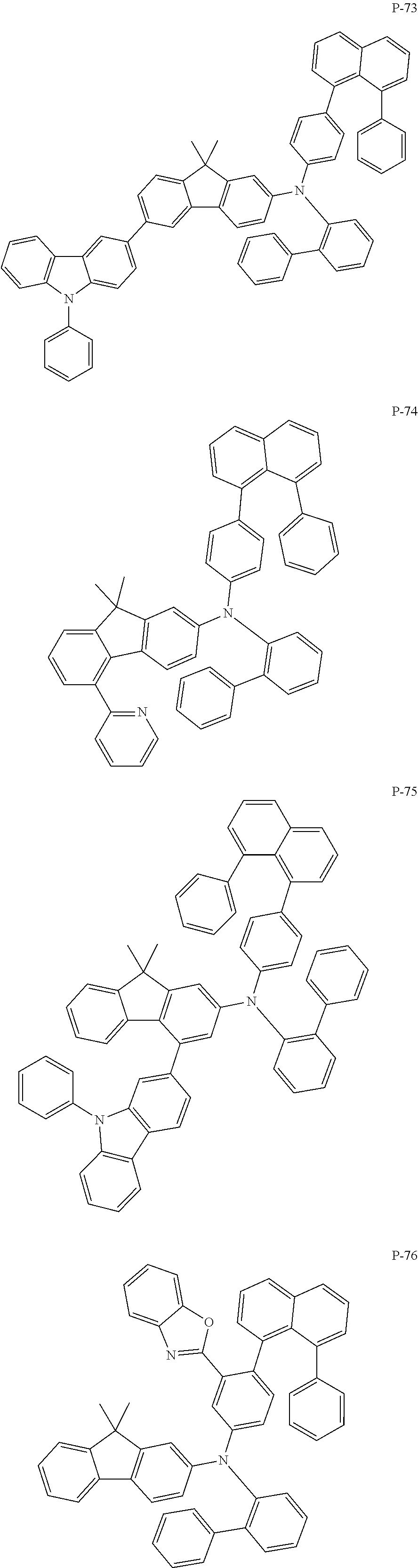 [SMILES: CC1(C)c2ccc(-c3ccc4c(c3)c3ccccc3n4-c3ccccc3)cc2-c2ccc(N(c3ccc(-c4cccc5cccc(-c6ccccc6)c45)cc3)c3ccccc3-c3ccccc3)cc21 ; CC1(C)c2cc(N(c3ccc(-c4cccc5cccc(-c6ccccc6)c45)cc3)c3ccccc3-c3ccccc3)ccc2-c2c(-c3ccccn3)cccc21 ; CC1(C)c2ccccc2-c2c(-c3ccc4c5ccccc5n(-c5ccccc5)c4c3)cc(N(c3ccc(-c4cccc5cccc(-c6ccccc6)c45)cc3)c3ccccc3-c3ccccc3)cc21 ; CC1(C)c2ccccc2-c2ccc(N(c3ccc(-c4cccc5cccc(-c6ccccc6)c45)c(-c4nc5ccccc5o4)c3)c3ccccc3-c3ccccc3)cc21]

In another aspect, the present invention provides a method for reusing a compound of Formula 1 comprising:
recovering a crude organic light emitting material comprising the compound of Formula 1 of from a deposition apparatus used in the process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.

The step of removing impurities from the crude organic light emitting material recovered from the deposition apparatus may preferably comprise performing a pre-purification process to obtain a purity of 98% or more by recrystallization in a recrystallization solvent.

The recrystallization solvent may be preferably a polar solvent having a polarity index (PI) of 5.5 to 7.2.

The recrystallization solvent may preferably be used by mixing a polar solvent having a polarity value of 5.5 to 7.2 and a non-polar solvent having a polarity value of 2.0 to 4.7.

When a mixture of a polar solvent and a non-polar solvent is used, the recrystallization solvent may be used in an amount of 15% (v/v) or less of the non-polar solvent compared to the polar solvent.

The recrystallization solvent may preferably be used by mixing N-Methylpyrrolidone (NMP) single solvent; or a polar solvent mixed any one selected from the group consisting of 1,3-Dimethyl-2-imidazolidinone, 2-pyrrolidone, N, N-Dimethyl formamide, Dimethyl acetamide, and Dimethyl sulfoxide to the N-Methylpyrrolidone; or alone; or mixed non-polar solvents; selected from the group consisting of Toluene, Dichloromethane (DCM), Dichloroethane (DCE), Tetrahydrofuran (THF), Chloroform, Ethyl acetate and Butanone; or a polar solvent and a non-polar solvent.

The pre-purification process may comprise a step of precipitating crystals of by cooling to 0° C. to 5° C. after dissolving the crude organic light emitting material recovered from the deposition apparatus in a polar solvent at 90° C. to 120° C.

The pre-purification process may comprise a step of precipitating crystals by cooling to 35° C. to 40° C., adding a non-polar solvent, and then cooling to 0° C. to 5° C. after dissolving the crude organic light emitting material recovered from the deposition apparatus in a polar solvent at 90° C. to 120° C.

The pre-purification process may comprise a step of precipitating crystals while concentrating the solvent and removing the non-polar solvent, after dissolving the crude organic light emitting material recovered from the deposition apparatus in a non-polar solvent.

The pre-purification process may comprise a step of recrystallizing again with a non-polar solvent after recrystallizing first with a polar solvent.

The step of purifying the recovered impurities to a purity of 99.9% or higher may comprise performing an adsorption separation process to adsorb and remove impurities by adsorbing on the adsorbent.

The adsorbent may be activated carbon, silica gel, alumina, or a material for known adsorption purposes.

The step of purifying the recovered impurities to a purity of 99.9% or higher may comprise performing sublimation purification.

Referring to FIG. 1, the organic electronic element (100) according to the present invention comprises a first electrode (110), a second electrode (170), an organic material layer formed between the first electrode (110) and the second electrode (170) and a light efficiency enhancing layer, wherein the light efficiency enhancing layer (180) is formed on a surface not in contact with the organic material layer among both surfaces of the first electrode (110) or the second electrode (170), wherein the organic layer or light efficiency enhancing layer comprises a single compound or 2 or more compounds represented by Formula 1.

Wherein, the first electrode (110) may be an anode or a positive electrode, and the second electrode (170) may be a cathode or a negative electrode. In the case of an inverted organic electronic element, the first electrode may be a cathode, and the second electrode may be an anode.

The organic material layer may sequentially comprise a hole injection layer (120), a hole transport layer (130), an emitting layer (140), an electron transport layer (150), and an electron injection layer (160) on the first electrode (110). Here, the remaining layers except the emitting layer (140) may not be formed. The organic material layer may further comprise a hole blocking layer, an electron blocking layer, an emitting-auxiliary layer (220), a buffer layer (210), etc., and the electron transport layer (150) and the like may serve as a hole blocking layer (see FIG. 2).

Also, the organic electronic element according to an embodiment of the present invention may further include a protective layer.

The organic electronic element according to an embodiment of the present invention may further comprise a protective layer.

The compound according to an embodiment of the present invention may be used as a material for the organic material layer, that is a hole injection layer (120), a hole transport layer (130), an emitting-auxiliary layer (220), an electron transport auxiliary layer, an electron transport layer (150), an electron injection layer (160), or a host or dopant of an emitting layer (140), or the light efficiency enhancing layer. Preferably, for example, the compound according to Formula 1 of the present invention can be used as a hole transport layer material.

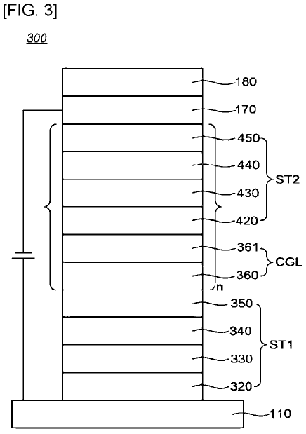

The organic material layer may comprise 2 or more stacks comprising a hole transport layer, an emitting layer and an electron transport layer sequentially formed on the anode, and may further comprise a charge generation layer formed between the 2 or more stacks (see FIG. 3).

Otherwise, even if the same core is used, the band gap, the electrical characteristics, the interface characteristics, etc. may vary depending on which substituent is bonded at which position, therefore the choice of core and the combination of sub-substituents associated therewith is also very important.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer comprising the hole injection layer (120), the hole transport layer (130), the emitting layer (140), the electron transport layer (150), and the electron injection layer (160) is formed thereon, and then depositing a material usable as a cathode thereon can be manufactured.

Additionally, in the present invention, the organic material layer is formed by any one of a spin coating process, a nozzle printing process, an inkjet printing process, a slot coating process, a dip coating process, and a roll-to-roll process.

As another specific example, the present invention provides a hole transport layer composition comprising the compound represented by Formula 1.

Also, a mixture of the same or different compounds of the compound represented by Formula 1 is used in the hole transport layer.

Also, the present invention also provides an electronic device comprising a display device comprising the organic electronic element; and a control unit for driving the display device.

According to another aspect, the present invention provides a display device wherein the organic electronic element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, Synthesis examples of the compound represented by Formula 1 of the present invention and preparation examples of the organic electronic element of the present invention will be described in detail by way of example, but are not limited to the following examples.

### Synthesis Example 1

The compound (final products) represented by Formula 1 according to the present invention can be synthesized as shown in Scheme 1, but is not limited thereto.

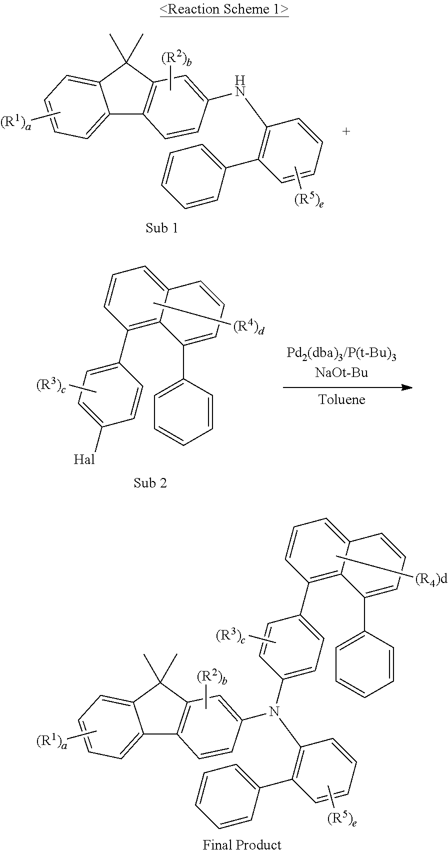

Wherein,
1) Hal is I, Br or Cl,
2) R¹, R², R³, R⁴, R⁵, a, b, c, d and e are the same as defined above.

### I. Synthesis of Sub-1

Sub 1 of Reaction Scheme 1 is synthesized through the reaction route of Reaction Scheme 2, but is not limited thereto. Hal is I, Br or Cl.

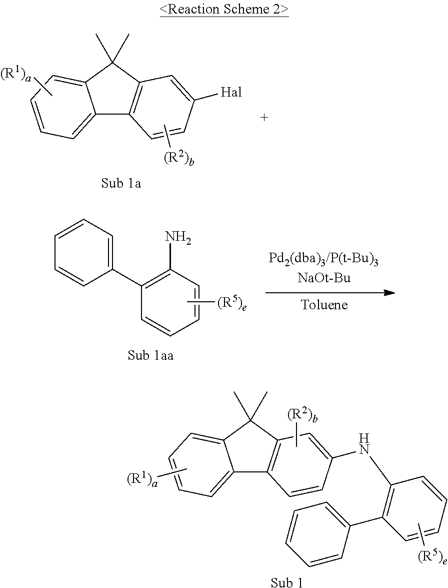

### 1. Synthesis Example of Sub 1-4

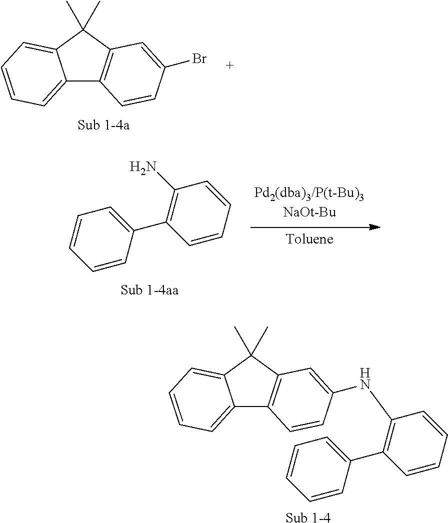

Sub 1-4a (50.0 g, 183.0 mmol) was dissolved in toluene (915 mL) in a round bottom flask, and Sub 1-4aa (29.9 g, 183.0 mmol), Pd₂(dba)₃ (5.0 g, 5.5 mmol), P(t-Bu)₃ (2.2 g, 11.0 mmol), NaOt-Bu (35.2 g, 366.1 mmol) were added and stirred at 120° C. When the reaction was completed, extraction was performed with CH₂Cl₂ and water, the organic layer was dried with MgSO₄, concentrated, and the resulting compound was recrystallized using a silicagel column to obtain 48.8 g of product. (yield: 73.8%)

### 2. Synthesis Example of Sub1-6

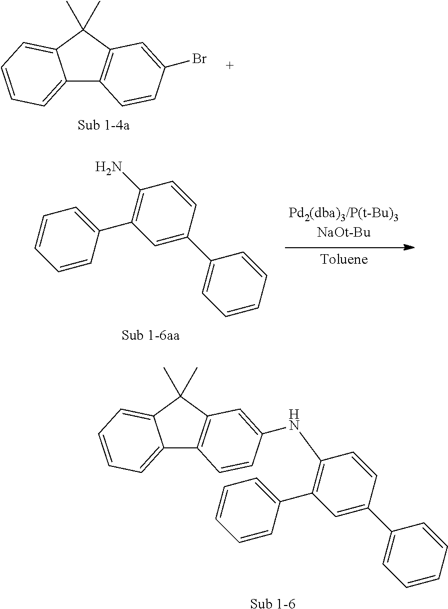 [SMILES: CC1(C)c2ccccc2-c2ccc(Br)cc21 ; Nc1ccc(-c2ccccc2)cc1-c1ccccc1 ; CC1(C)c2ccccc2-c2ccc(Nc3ccc(-c4ccccc4)cc3-c3ccccc3)cc21 ; "Sub 1-6"]

Sub 1-4a (50.0 g, 183.0 mmol) and Sub 1-6aa (43.4 g, 183.0 mmol), Pd₂(dba)₃ (5.0 g, 5.5 mmol), P(t-Bu)₃ (2.2 g, 11.0 mmol), NaOt-Bu (35.2 g, 366.1 mmol), toluene (915 mL) were tested in the same manner as Sub 1-4 in a round bottom flask to obtain 57.3 g of product. (Yield: 71.5%)

### 3. Synthesis Example of Sub1-13

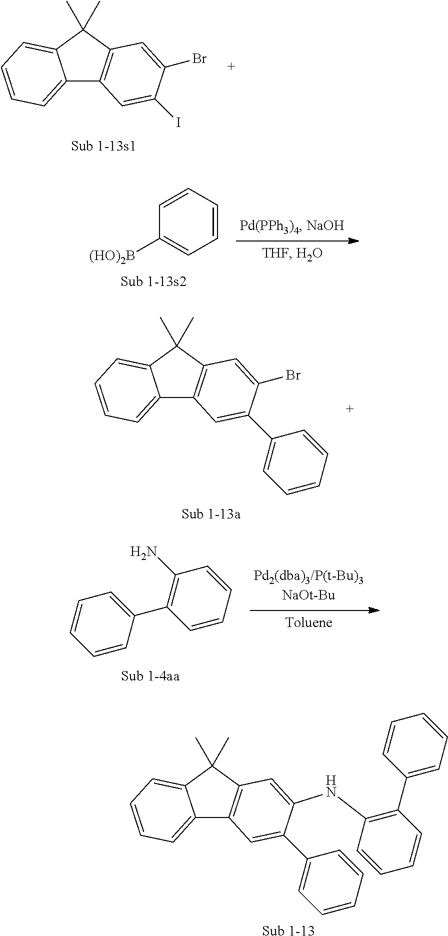 [SMILES: CC1(C)c2ccccc2-c2cc(I)c(Br)cc21 ; OB(O)c1ccccc1 ; CC1(C)c2ccccc2-c2cc(-c3ccccc3)c(Br)cc21 ; Nc1ccccc1-c1ccccc1 ; CC1(C)c2ccccc2-c2cc(-c3ccccc3)c(Nc3ccccc3-c3ccccc3)cc21 ; "Sub 1-13"]

### 1) Synthesis Sub1-13a

Sub 1-13s1 (50.0 g, 125.3 mmol) was dissolved in THF (626 mL) in a round bottom flask, Sub 1-13s2 (15.3 g, 125.3 mmol), Pd(PPh₃)₄ (8.7 g, 7.5 mmol), NaOH (15.0 g, 375.9 mmol), Water (313 ml) were added and the reaction proceeds at 80° C. When the reaction was completed, extraction was performed with CH₂Cl₂ and water, the organic layer was dried with MgSO₄, concentrated, and the resulting compound was recrystallized using a silicagel column to obtain 36.2 g of product. (yield: 82.7%)

### 2) Synthesis Sub1-13

Sub 1-13a (30.0 g, 85.9 mmol) and Sub 1-4aa (14.0 g, 85.9 mmol), Pd₂(dba)₃ (2.4 g, 2.6 mmol), P(t-Bu)₃ (1.0 g, 5.2 mmol), NaOt-Bu (16.5 g, 171.8 mmol), toluene (429 mL) were tested in the same manner as Sub 1-4 in a round bottom flask to obtain 25.9 g of product. (Yield: 68.9%)

### 4. Synthesis Example of Sub1-17

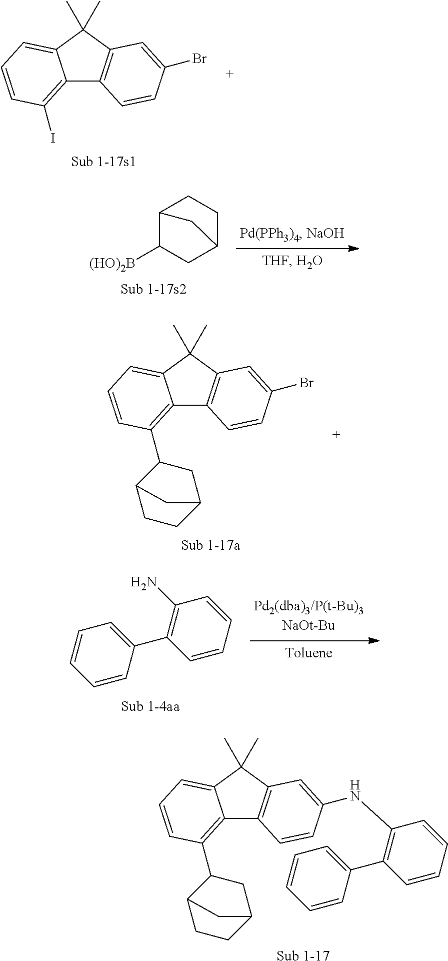 [SMILES: CC1(C)c2cc(Br)ccc2-c2c(I)cccc21 ; OB(O)C1CC2CCC1C2 ; CC1(C)c2cc(Br)ccc2-c2c(C3CC4CCC3C4)cccc21 ; Nc1ccccc1-c1ccccc1 ; CC1(C)c2cc(Nc3ccccc3-c3ccccc3)ccc2-c2c(C3CC4CCC3C4)cccc21 ; "Sub 1-17"]

### 1) Synthesis Sub1-17a

Sub 1-17s1 (50.0 g, 125.3 mmol) was dissolved in THF (626 mL) in a round bottom flask, Sub 1-17s2 (17.5 g, 125.3 mmol), Pd(PPh₃)₄ (8.7 g, 7.5 mmol), NaOH (15.0 g, 375.9 mmol), water (313 ml) were added and 35.1 g of product was obtained by performing the same manner as Sub 1-13a. (Yield: 76.2%)

### 2) Synthesis Sub1-17

Sub 1-17a (30.0 g, 81.7 mmol) and Sub 1-4aa (13.4 g, 81.7 mmol), Pd₂(dba)₃ (2.2 g, 2.5 mmol), P(t-Bu)₃ (1.0 g, 4.9 mmol), NaOt-Bu (15.7 g, 163.3 mmol), toluene (408 mL) were tested in the same manner as Sub 1-4 in a round bottom flask to obtain 25.1 g of product. (Yield: 67.5%)

### 5. Synthesis Example of Sub1-52

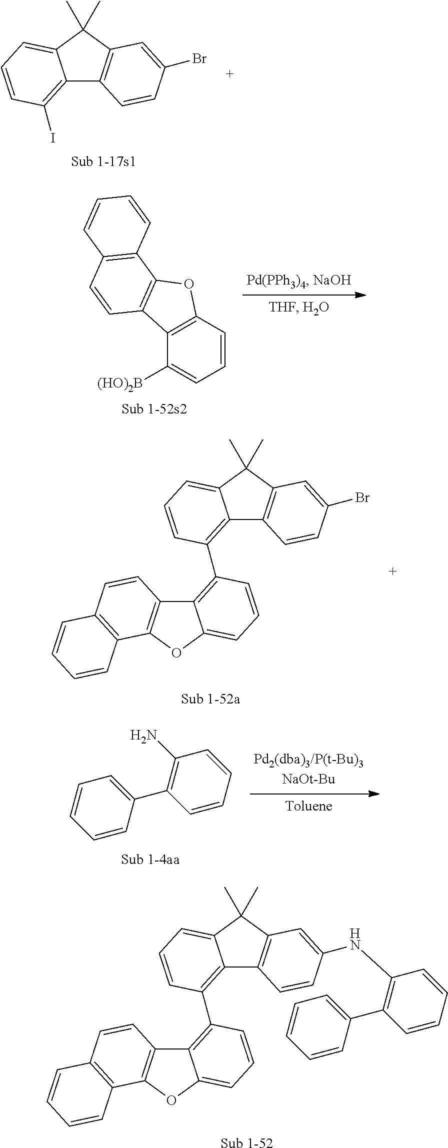 [SMILES: CC1(C)c2cc(Br)ccc2-c2c(I)cccc21 ; OB(O)c1cccc2oc3c4ccccc4ccc3c12 ; CC1(C)c2cc(Br)ccc2-c2c(-c3cccc4oc5c6ccccc6ccc5c34)cccc21 ; Nc1ccccc1-c1ccccc1 ; CC1(C)c2cc(Nc3ccccc3-c3ccccc3)ccc2-c2c(-c3cccc4oc5c6ccccc6ccc5c34)cccc21 ; "Sub 1-52"]

### 1) Synthesis Sub1-52a

Sub 1-17s1 (50.0 g, 125.3 mmol) was dissolved in THF (626 mL) in a round bottom flask, Sub 1-52s2 (32.8 g, 125.3 mmol), Pd(PPh₃)₄ (8.7 g, 7.5 mmol), NaOH (15.0 g, 375.9 mmol), water (313 ml) were added and 48.1 g of product was obtained by performing the same manner as Sub 1-13a. (Yield: 78.5%)

### 2) Synthesis Sub1-52

Sub 1-52a (30.0 g, 61.3 mmol) and Sub 1-4aa (15.5 g, 61.3 mmol), Pd₂(dba)₃ (1.7 g, 1.8 mmol), P(t-Bu)₃ (0.7 g, 3.7 mmol), NaOt-Bu (11.8 g, 122.6 mmol), toluene (306 mL) were tested in the same manner as Sub 1-4 in a round bottom flask to obtain 24.2 g of product. (Yield: 68.3%)

Compounds belonging to Sub 1 may be, but are not limited to, the following compounds, and Table 1 shows the FD-MS (Field Desorption-Mass Spectrometry) values of the compounds belonging to Sub 1.

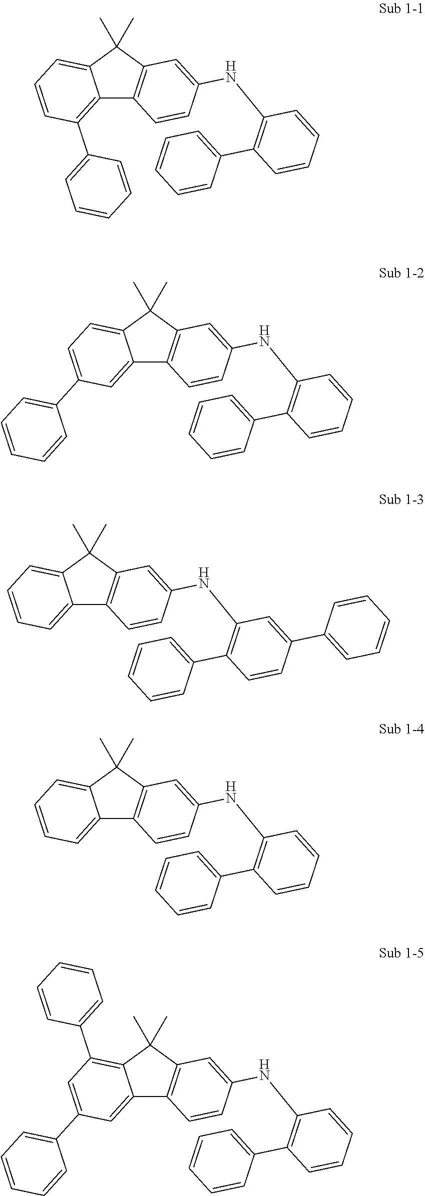

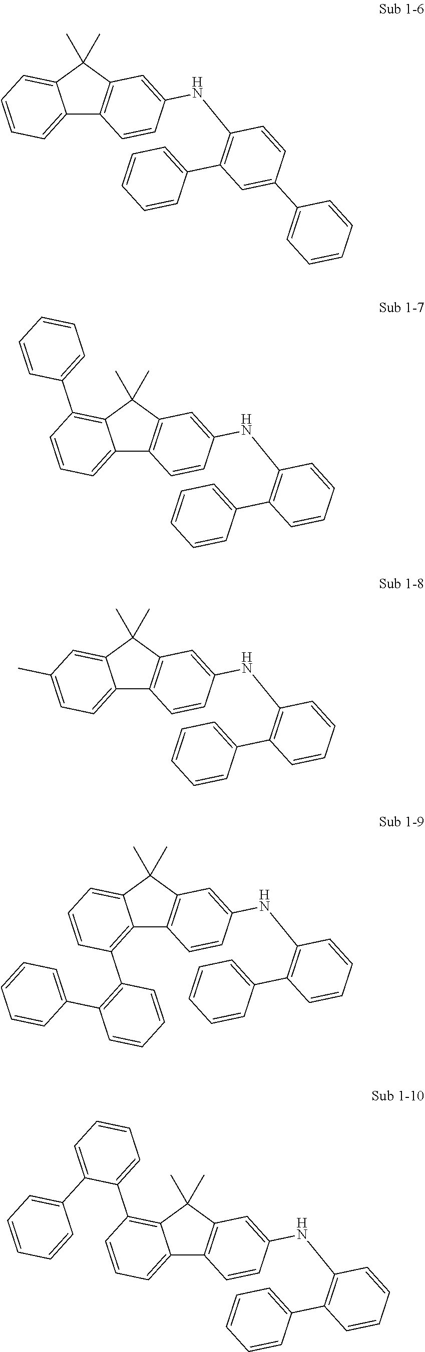 [SMILES: CC1(C)c2ccccc2-c2ccc(Nc3ccc(-c4ccccc4)cc3-c3ccccc3)cc21 ; CC1(C)c2cc(Nc3ccccc3-c3ccccc3)ccc2-c2cccc(-c3ccccc3)c21 ; Cc1ccc2c(c1)C(C)(C)c1cc(Nc3ccccc3-c3ccccc3)ccc1-2 ; CC1(C)c2cc(Nc3ccccc3-c3ccccc3)ccc2-c2c(-c3ccccc3-c3ccccc3)cccc21 ; CC1(C)c2cc(Nc3ccccc3-c3ccccc3)ccc2-c2cccc(-c3ccccc3-c3ccccc3)c21]

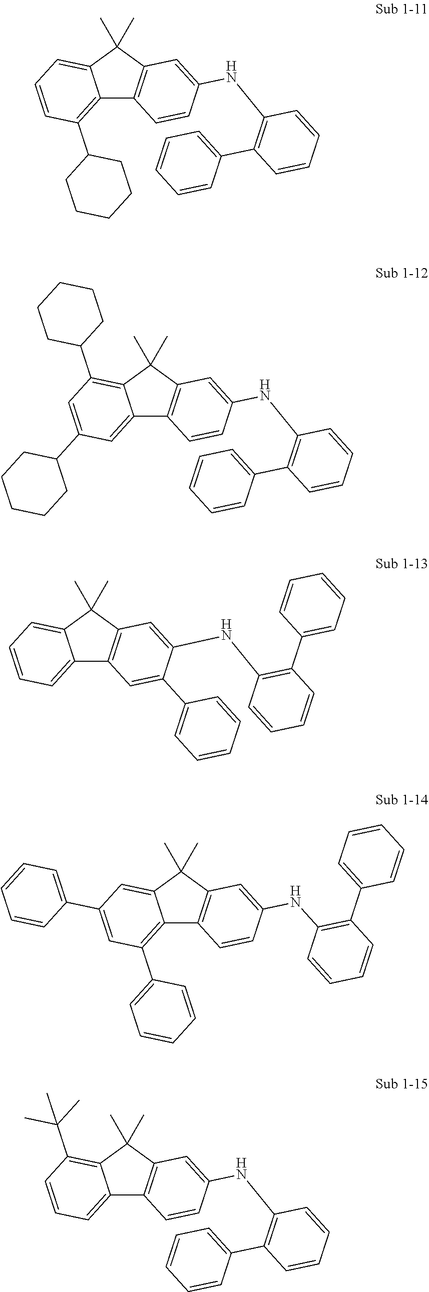 [SMILES: CC1(C)c2cc(Nc3ccccc3-c3ccccc3)ccc2-c2c(C3CCCCC3)cccc21 ; CC1(C)c2cc(Nc3ccccc3-c3ccccc3)ccc2-c2cc(C3CCCCC3)cc(C3CCCCC3)c21 ; CC1(C)c2ccccc2-c2cc(-c3ccccc3)c(Nc3ccccc3-c3ccccc3)cc21 ; CC1(C)c2cc(Nc3ccccc3-c3ccccc3)ccc2-c2c(-c3ccccc3)cc(-c3ccccc3)cc21 ; CC(C)(C)c1cccc2c1C(C)(C)c1cc(Nc3ccccc3-c3ccccc3)ccc1-2]

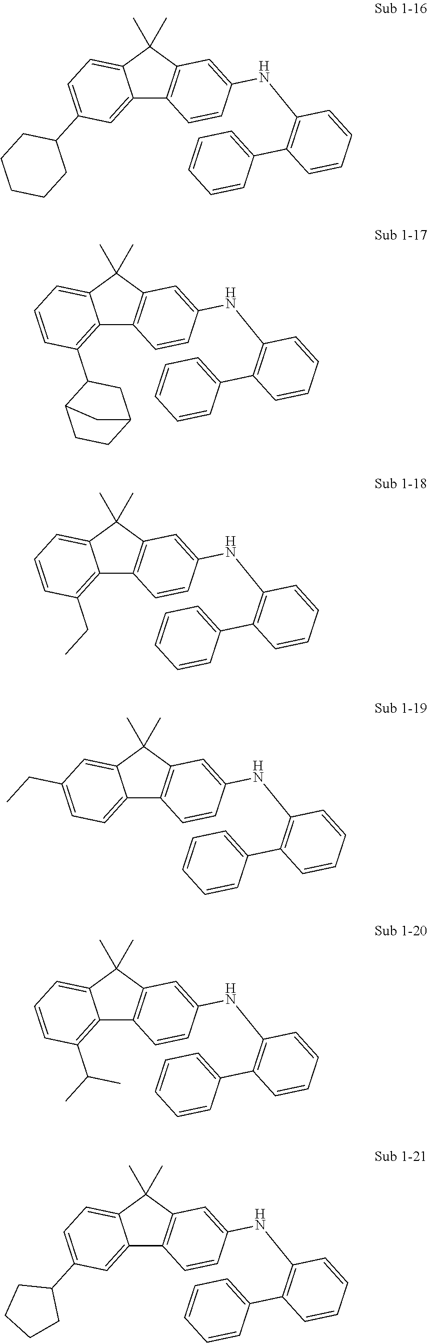 [SMILES: CC1(C)c2ccc(C3CCCCC3)cc2-c2ccc(Nc3ccccc3-c3ccccc3)cc21 ; CC1(C)c2cc(Nc3ccccc3-c3ccccc3)ccc2-c2c(C3CC4CCC3C4)cccc21 ; CCc1cccc2c1-c1ccc(Nc3ccccc3-c3ccccc3)cc1C2(C)C ; CCc1ccc2c(c1)C(C)(C)c1cc(Nc3ccccc3-c3ccccc3)ccc1-2 ; CC(C)c1cccc2c1-c1ccc(Nc3ccccc3-c3ccccc3)cc1C2(C)C ; CC1(C)c2ccc(C3CCCC3)cc2-c2ccc(Nc3ccccc3-c3ccccc3)cc21]

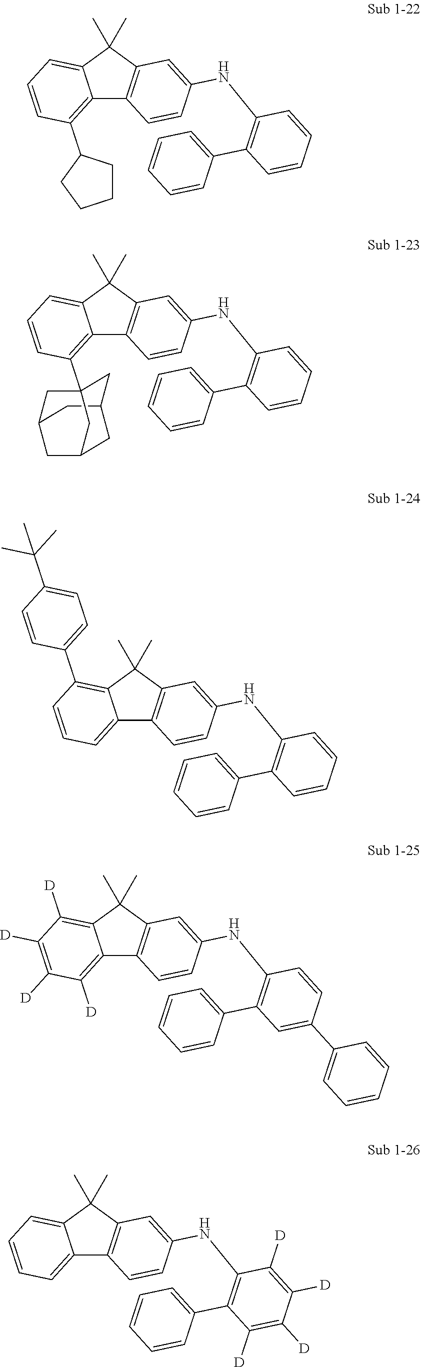 [SMILES: CC1(C)c2cc(Nc3ccccc3-c3ccccc3)ccc2-c2c(C3CCCC3)cccc21 ; CC1(C)c2cc(Nc3ccccc3-c3ccccc3)ccc2-c2c(C34CC5CC(CC(C5)C3)C4)cccc21 ; CC(C)(C)c1ccc(-c2cccc3c2C(C)(C)c2cc(Nc4ccccc4-c4ccccc4)ccc2-3)cc1 ; [2H]c1c([2H])c([2H])c2c(c1[2H])-c1ccc(Nc3ccc(-c4ccccc4)cc3-c3ccccc3)cc1C2(C)C ; [2H]c1c([2H])c([2H])c(-c2ccccc2)c(Nc2ccc3c(c2)C(C)(C)c2ccccc2-3)c1[2H]]

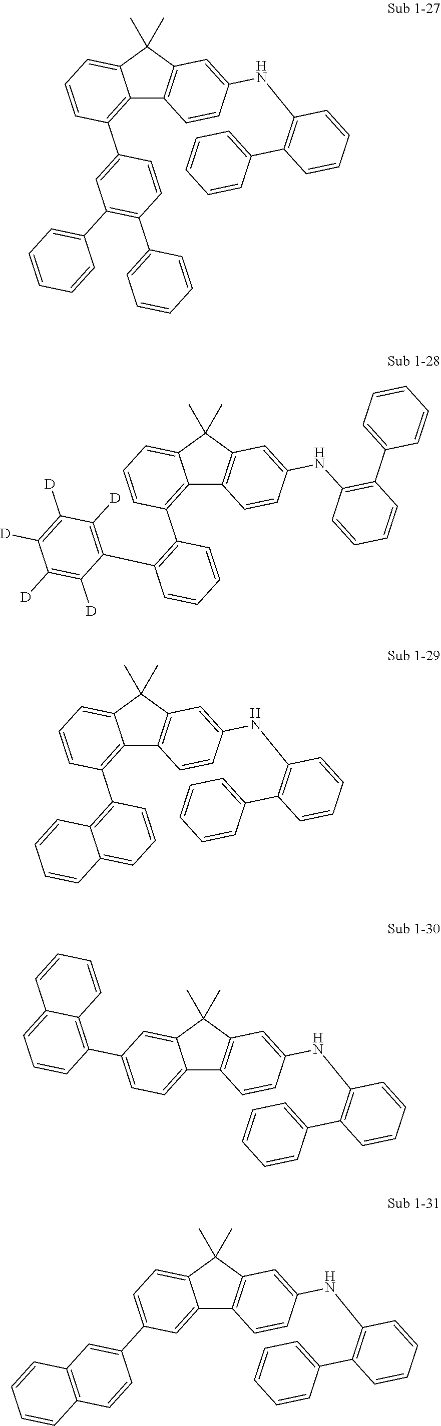 [SMILES: CC1(C)c2cc(Nc3ccccc3-c3ccccc3)ccc2-c2c(-c3ccc(-c4ccccc4)c(-c4ccccc4)c3)cccc21 ; [2H]c1c([2H])c([2H])c(-c2ccccc2-c2cccc3c2-c2ccc(Nc4ccccc4-c4ccccc4)cc2C3(C)C)c([2H])c1[2H] ; CC1(C)c2cc(Nc3ccccc3-c3ccccc3)ccc2-c2c(-c3cccc4ccccc34)cccc21 ; CC1(C)c2cc(Nc3ccccc3-c3ccccc3)ccc2-c2ccc(-c3cccc4ccccc34)cc21 ; CC1(C)c2ccc(-c3ccc4ccccc4c3)cc2-c2ccc(Nc3ccccc3-c3ccccc3)cc21]

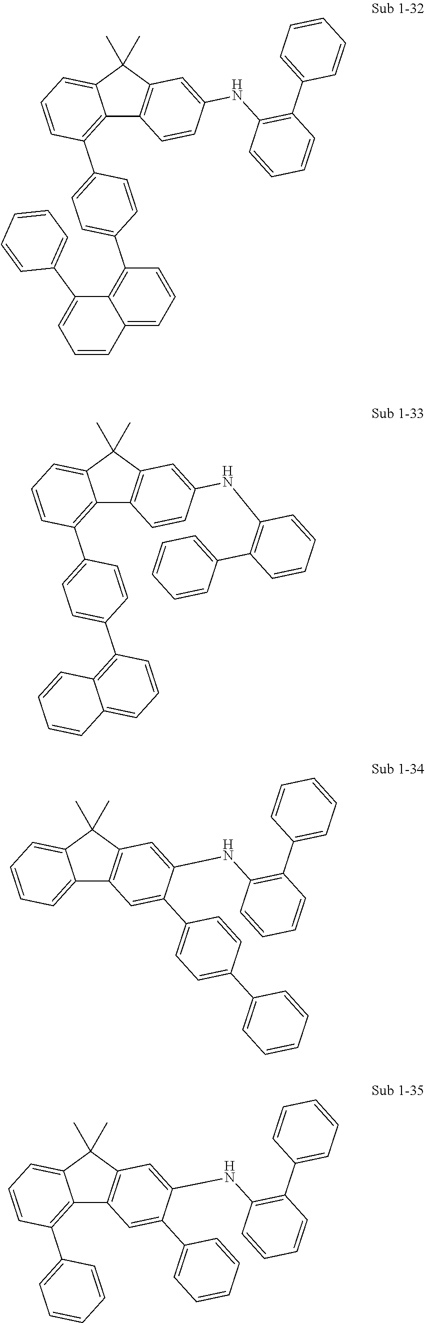 [SMILES: CC1(C)c2cc(Nc3ccccc3-c3ccccc3)ccc2-c2c(-c3ccc(-c4cccc5cccc(-c6ccccc6)c45)cc3)cccc21 ; CC1(C)c2cc(Nc3ccccc3-c3ccccc3)ccc2-c2c(-c3ccc(-c4cccc5ccccc45)cc3)cccc21 ; CC1(C)c2ccccc2-c2cc(-c3ccc(-c4ccccc4)cc3)c(Nc3ccccc3-c3ccccc3)cc21 ; CC1(C)c2cc(Nc3ccccc3-c3ccccc3)c(-c3ccccc3)cc2-c2c(-c3ccccc3)cccc21]

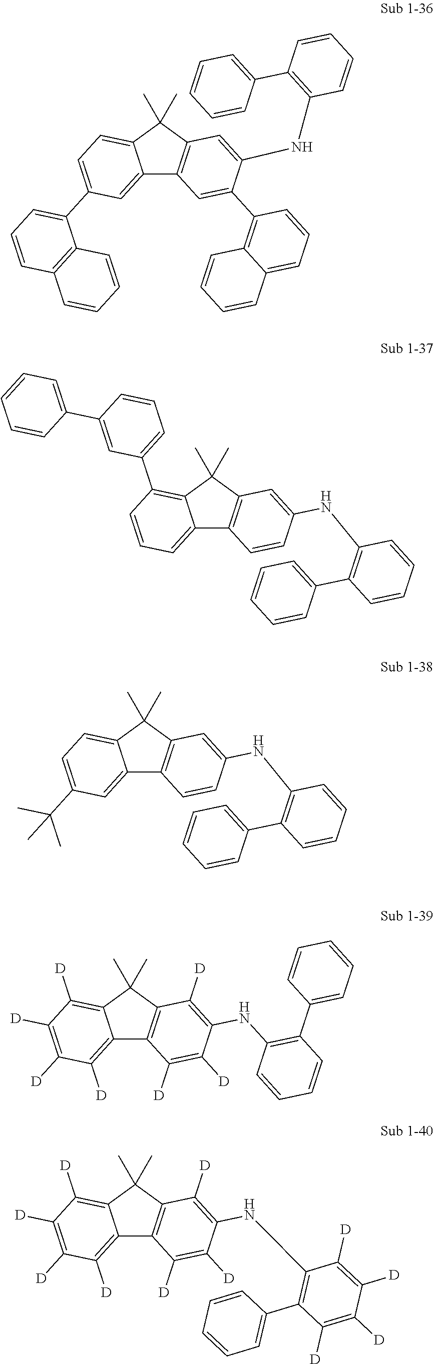 [SMILES: CC1(C)c2ccc(-c3cccc4ccccc34)cc2-c2cc(-c3cccc4ccccc34)c(Nc3ccccc3-c3ccccc3)cc21 ; CC1(C)c2cc(Nc3ccccc3-c3ccccc3)ccc2-c2cccc(-c3cccc(-c4ccccc4)c3)c21 ; CC(C)(C)c1ccc2c(c1)-c1ccc(Nc3ccccc3-c3ccccc3)cc1C2(C)C ; [2H]c1c([2H])c([2H])c2c(c1[2H])-c1c([2H])c([2H])c(Nc3ccccc3-c3ccccc3)c([2H])c1C2(C)C ; [2H]c1c([2H])c([2H])c(-c2ccccc2)c(Nc2c([2H])c([2H])c3c(c2[2H])C(C)(C)c2c([2H])c([2H])c([2H])c([2H])c2-3)c1[2H]]

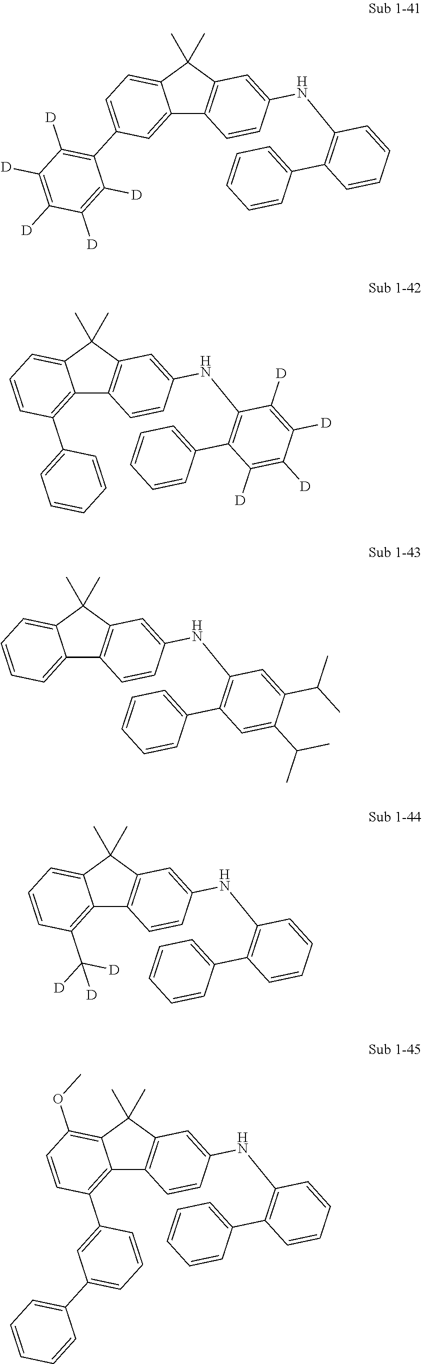 [SMILES: [2H]c1c([2H])c([2H])c(-c2ccc3c(c2)-c2ccc(Nc4ccccc4-c4ccccc4)cc2C3(C)C)c([2H])c1[2H] ; [2H]c1c([2H])c([2H])c(-c2ccccc2)c(Nc2ccc3c(c2)C(C)(C)c2cccc(-c4ccccc4)c2-3)c1[2H] ; CC(C)c1cc(Nc2ccc3c(c2)C(C)(C)c2ccccc2-3)c(-c2ccccc2)cc1C(C)C ; [2H]C([2H])([2H])c1cccc2c1-c1ccc(Nc3ccccc3-c3ccccc3)cc1C2(C)C ; COc1ccc(-c2cccc(-c3ccccc3)c2)c2c1C(C)(C)c1cc(Nc3ccccc3-c3ccccc3)ccc1-2]

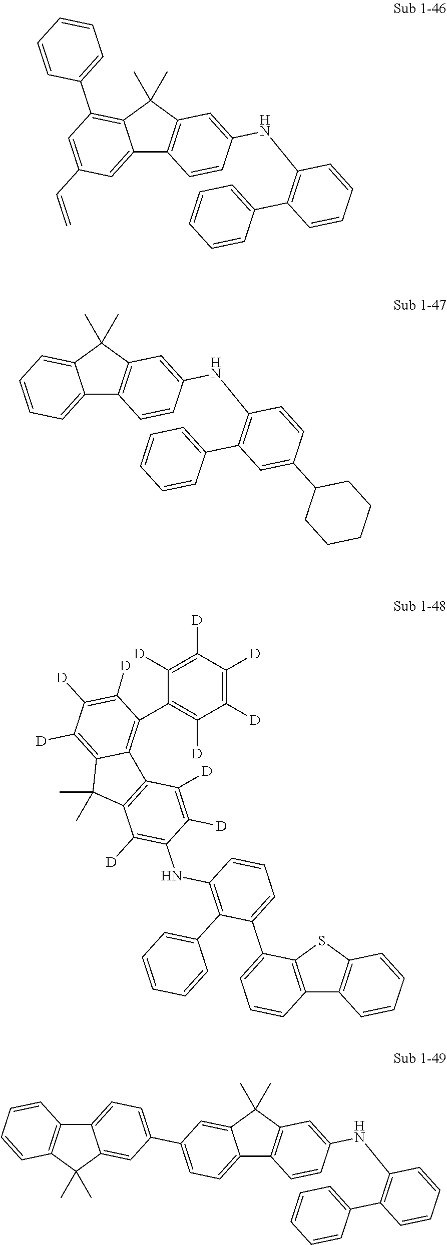 [SMILES: C=Cc1cc(-c2ccccc2)c2c(c1)-c1ccc(Nc3ccccc3-c3ccccc3)cc1C2(C)C ; CC1(C)c2ccccc2-c2ccc(Nc3ccc(C4CCCCC4)cc3-c3ccccc3)cc21 ; [2H]c1c([2H])c([2H])c(-c2c([2H])c([2H])c([2H])c3c2-c2c([2H])c([2H])c(Nc4cccc(-c5cccc6c5sc5ccccc56)c4-c4ccccc4)c([2H])c2C3(C)C)c([2H])c1[2H] ; CC1(C)c2ccccc2-c2ccc(-c3ccc4c(c3)C(C)(C)c3cc(Nc5ccccc5-c5ccccc5)ccc3-4)cc21]

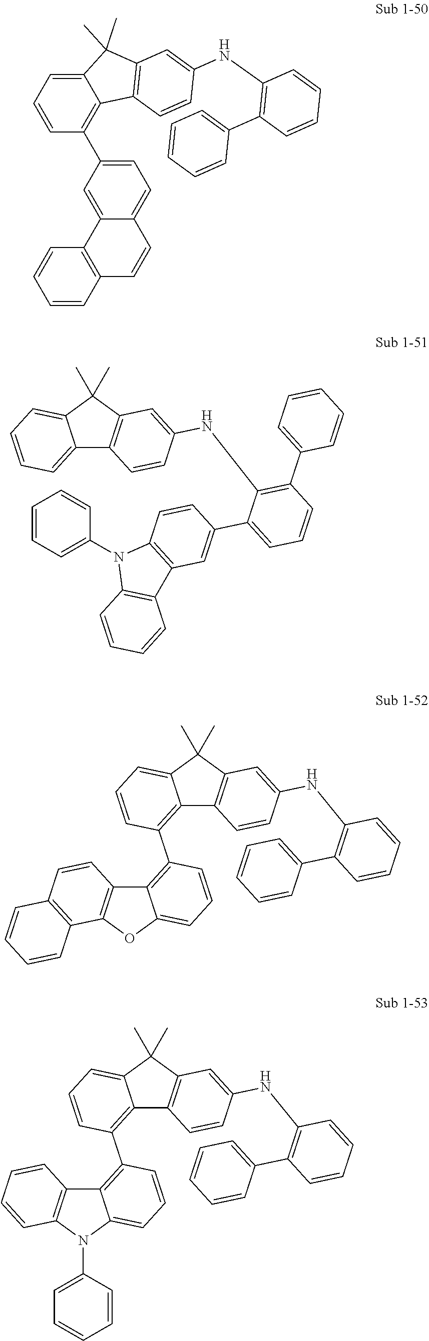 [SMILES: CC1(C)c2cc(Nc3ccccc3-c3ccccc3)ccc2-c2c(-c3ccc4ccc5ccccc5c4c3)cccc21 ; CC1(C)c2ccccc2-c2ccc(Nc3c(-c4ccccc4)cccc3-c3ccc4c(c3)c3ccccc3n4-c3ccccc3)cc21 ; CC1(C)c2cc(Nc3ccccc3-c3ccccc3)ccc2-c2c(-c3cccc4oc5c6ccccc6ccc5c34)cccc21 ; CC1(C)c2cc(Nc3ccccc3-c3ccccc3)ccc2-c2c(-c3cccc4c3c3ccccc3n4-c3ccccc3)cccc21]

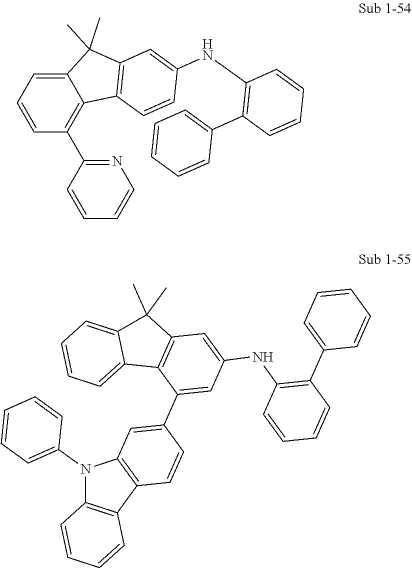 [SMILES: CC1(C)c2cc(Nc3ccccc3-c3ccccc3)ccc2-c2c(-c3ccccn3)cccc21 ; CC1(C)c2ccccc2-c2c(-c3ccc4c5ccccc5n(-c5ccccc5)c4c3)cc(Nc3ccccc3-c3ccccc3)cc21]

TABLE 1

| Com- | | Com- | |
|---|---|---|---|
| pound | FD-MS | pound | FD-MS |
| Sub 1-1 | m/z = 437.21(C₃₃H₂₇N = 437.59) | Sub 1-2 | m/z = 437.21(C₃₃H₂₇N = 437.59) |
| Sub 1-3 | m/z = 437.21(C₃₃H₂₇N = 437.59) | Sub 1-4 | m/z = 361.18(C₂₇H₂₃N = 361.49) |
| Sub 1-5 | m/z = 513.25(C₃₉H₃₁N = 513.68) | Sub 1-6 | m/z = 437.21(C₃₃H₂₇N = 437.59) |
| Sub 1-7 | m/z = 437.21(C₃₃H₂₇N = 437.59) | Sub 1-8 | m/z = 375.2(C₂₈H₂₅N = 375.51) |
| Sub 1-9 | m/z = 513.25(C₃₉H₃₁N = 513.68) | Sub 1-10 | m/z = 513.25(C₃₉H₃₁N = 513.68) |
| Sub 1-11 | m/z = 443.26(C₃₃H₃₃N = 443.63) | Sub 1-12 | m/z = 525.34(C₃₉H₄₃N = 525.78) |
| Sub 1-13 | m/z = 437.21(C₃₃H₂₇N = 437.59) | Sub 1-14 | m/z = 513.25(C₃₉H₃₁N = 513.68) |
| Sub 1-15 | m/z = 417.25(C₃₁H₃₁N = 417.6) | Sub 1-16 | m/z = 443.26(C₃₃H₃₃N = 443.63) |
| Sub 1-17 | m/z = 455.26(C₃₄H₃₃N = 455.64) | Sub 1-18 | m/z = 389.21(C₂₉H₂₇N = 389.54) |
| Sub 1-19 | m/z = 389.21(C₂₉H₂₇N = 389.54) | Sub 1-20 | m/z = 403.23(C₃₀H₂₉N = 403.57) |
| Sub 1-21 | m/z = 429.25(C₃₂H₃₁N = 429.61) | Sub 1-22 | m/z = 429.25(C₃₂H₃₁N = 429.61) |
| Sub 1-23 | m/z = 495.29(C₃₇H₃₇N = 495.71) | Sub 1-24 | m/z = 493.28(C₃₇H₃₅N = 493.69) |
| Sub 1-25 | m/z = 441.24(C₃₃H₂₃D₄N = 441.61) | Sub 1-26 | m/z = 365.21(C₂₇H₁₉D₄N = 365.51) |
| Sub 1-27 | m/z = 589.28(C₄₅H₃₅N = 589.78) | Sub 1-28 | m/z = 518.28(C₃₉H₂₆D₅N = 518.71) |
| Sub 1-29 | m/z = 487.23(C₃₇H₂₉N = 487.65) | Sub 1-30 | m/z = 487.23(C₃₇H₂₉N = 487.65) |
| Sub 1-31 | m/z = 487.23(C₃₇H₂₉N = 487.65) | Sub 1-32 | m/z = 639.29(C₄₉H₃₇N = 639.84) |
| Sub 1-33 | m/z = 563.26(C₄₃H₃₃N = 563.74) | Sub 1-34 | m/z = 513.25(C₃₉H₃₁N = 513.68) |
| Sub 1-35 | m/z = 513.25(C₃₉H₃₁N = 513.68) | Sub 1-36 | m/z = 613.28(C₄₇H₃₅N = 613.8) |
| Sub 1-37 | m/z = 513.25(C₃₉H₃₁N = 513.68) | Sub 1-38 | m/z = 417.25(C₃₁H₃₁N = 417.6) |
| Sub 1-39 | m/z = 368.23(C₂₇H₁₆D₇N = 368.53) | Sub 1-40 | m/z = 372.25(C₂₇H₁₂D₁₁N = 372.56) |
| Sub 1-41 | m/z = 442.25(C₃₃H₂₂D₅N = 442.62) | Sub 1-42 | m/z = 441.24(C₃₃H₂₃D₄N = 441.61) |
| Sub 1-43 | m/z = 445.28(C₃₃H₃₅N = 445.65) | Sub 1-44 | m/z = 378.22(C₂₈H₂₂D₃N = 378.53) |
| Sub 1-45 | m/z = 543.26(C₄₀H₃₃NO = 543.71) | Sub 1-46 | m/z = 463.23(C₃₅H₂₉N = 463.62) |
| Sub 1-47 | m/z = 443.26(C₃₃H₃₃N = 443.63) | Sub 1-48 | m/z = 630.3(C₄₅H₂₂D₁₁NS = 630.89) |
| Sub 1-49 | m/z = 553.28(C₄₂H₃₅N = 553.75) | Sub 1-50 | m/z = 537.25(C₄₁H₃₁N = 537.71) |
| Sub 1-51 | m/z = 602.27(C₄₅H₃₄N₂ = 602.78) | Sub 1-52 | m/z = 577.24(C₄₃H₃₁NO = 577.73) |
| Sub 1-53 | m/z = 602.27(C₄₅H₃₄N₂ = 602.78) | Sub 1-54 | m/z = 438.21(C₃₂H₂₆N₂ = 438.57) |
| Sub 1-55 | m/z = 602.27(C₄₅H₃₄N₂ = 602.78) | | |

### II. Synthesis of Sub-2

Sub 2 of Reaction Scheme 1 is synthesized through the reaction route of Reaction Scheme 3, but is not limited thereto. Hal is I, Br or Cl.

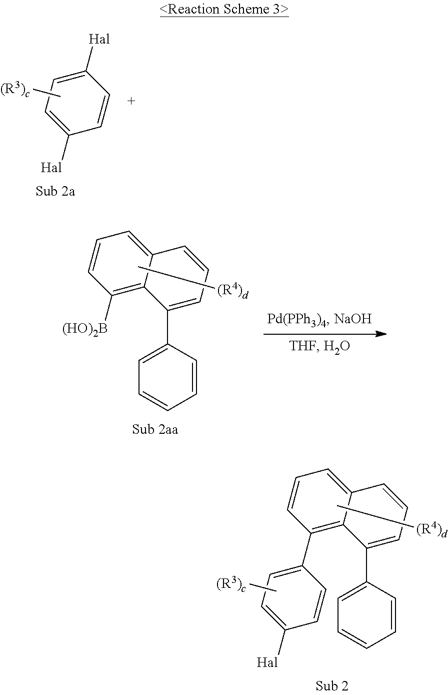

### 1. Synthesis Example of Sub 2-1

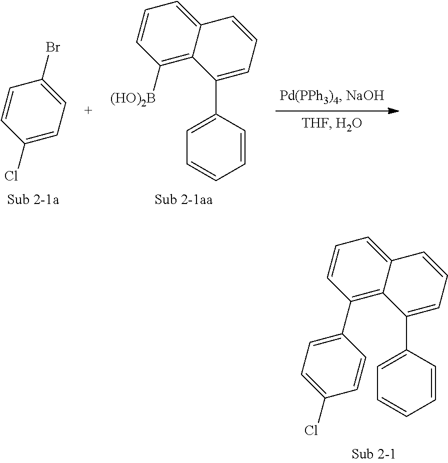 [SMILES: Clc1ccc(Br)cc1 ; OB(O)c1cccc2cccc(-c3ccccc3)c12 ; Clc1ccc(-c2cccc3cccc(-c4ccccc4)c23)cc1]

Sub 2-1a (50.0 g, 261.2 mmol) was dissolved in THF (1306 mL) in a round bottom flask, Sub 2-1aa (64.8 g, 261.2 mmol), Pd(PPh₃)₄ (18.1 g, 15.7 mmol), NaOH (31.3 g, 783.5 mmol), water (653 ml) were added and 66.8 g of product was obtained by performing the same manner as Sub 1-13a. (Yield: 81.2%)

### 2. Synthesis Example of Sub 2-6

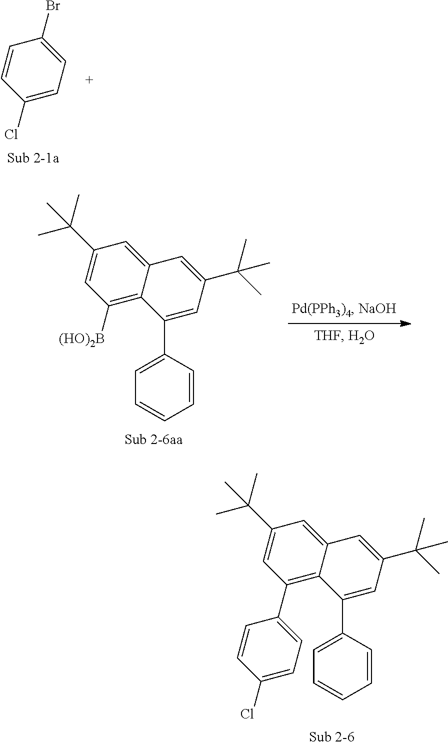 [SMILES: Clc1ccc(Br)cc1 ; CC(C)(C)c1cc(B(O)O)c2c(-c3ccccc3)cc(C(C)(C)C)cc2c1 ; CC(C)(C)c1cc(-c2ccccc2)c2c(-c3ccc(Cl)cc3)cc(C(C)(C)C)cc2c1]

Sub 2-1a (50.0 g, 261.2 mmol) was dissolved in THF (1306 mL) in a round bottom flask, Sub 2-6aa (94.1 g, 261.2 mmol), Pd(PPh₃)₄ (18.1 g, 15.7 mmol), NaOH (31.3 g, 783.5 mmol), water (653 ml) were added and 87.4 g of product was obtained by performing the same manner as Sub 1-13a. (Yield: 78.4%)

### 3. Synthesis Example of Sub 2-17

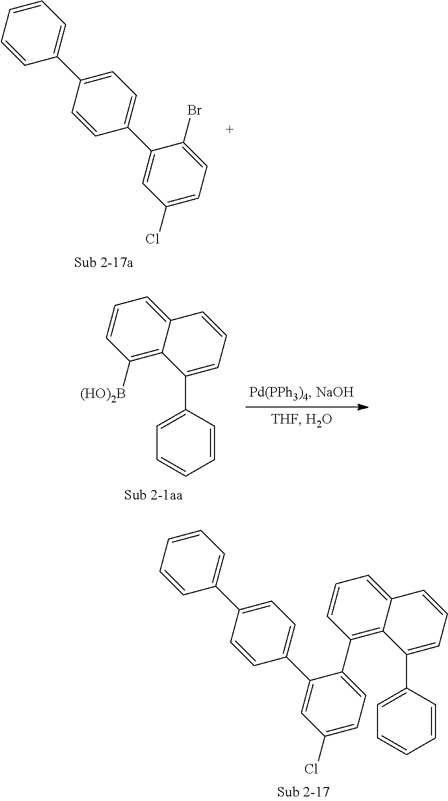 [SMILES: Clc1ccc(Br)c(-c2ccc(-c3ccccc3)cc2)c1 ; OB(O)c1cccc2cccc(-c3ccccc3)c12 ; Clc1ccc(-c2cccc3cccc(-c4ccccc4)c23)c(-c2ccc(-c3ccccc3)cc2)c1]

Sub 2-17a (50.0 g, 145.5 mmol) was dissolved in THF (727 mL) in a round bottom flask, Sub 2-1aa (36.1 g, 145.5 mmol), Pd(PPh₃)₄ (10.1 g, 8.7 mmol), NaOH (17.5 g, 436.5 mmol), water (364 ml) were added and 54.6 g of product was obtained by performing the same manner as Sub 1-13a. (Yield: 80.3%)

Compounds belonging to Sub 2 may be the following compounds, but are not limited thereto, and Table 2 shows the FD-MS (Field Desorption-Mass Spectrometry) values of the compounds belonging to Sub 2.

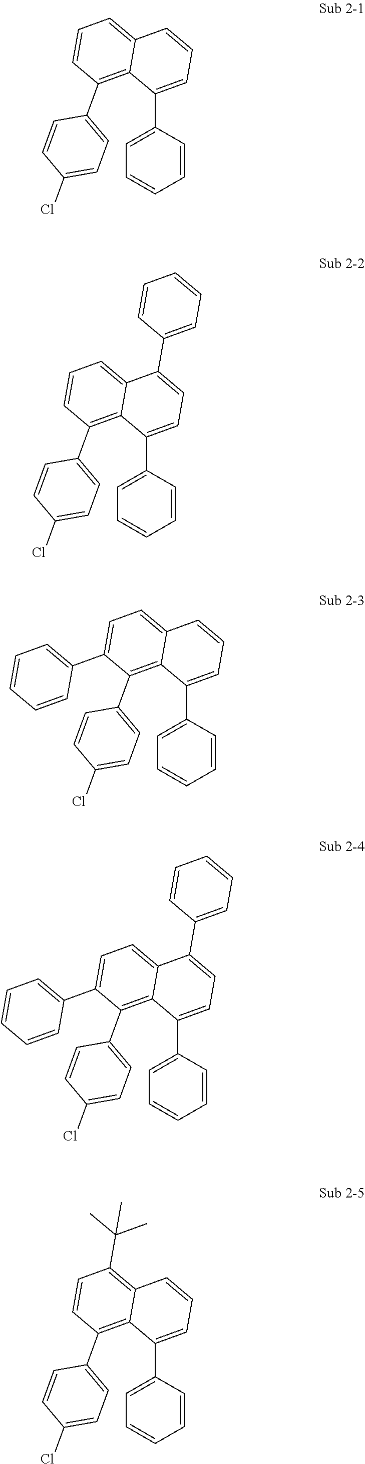 [SMILES: Clc1ccc(-c2cccc3cccc(-c4ccccc4)c23)cc1 ; Clc1ccc(-c2cccc3c(-c4ccccc4)ccc(-c4ccccc4)c23)cc1 ; Clc1ccc(-c2c(-c3ccccc3)ccc3cccc(-c4ccccc4)c23)cc1 ; Clc1ccc(-c2c(-c3ccccc3)ccc3c(-c4ccccc4)ccc(-c4ccccc4)c23)cc1 ; CC(C)(C)c1ccc(-c2ccc(Cl)cc2)c2c(-c3ccccc3)cccc12]

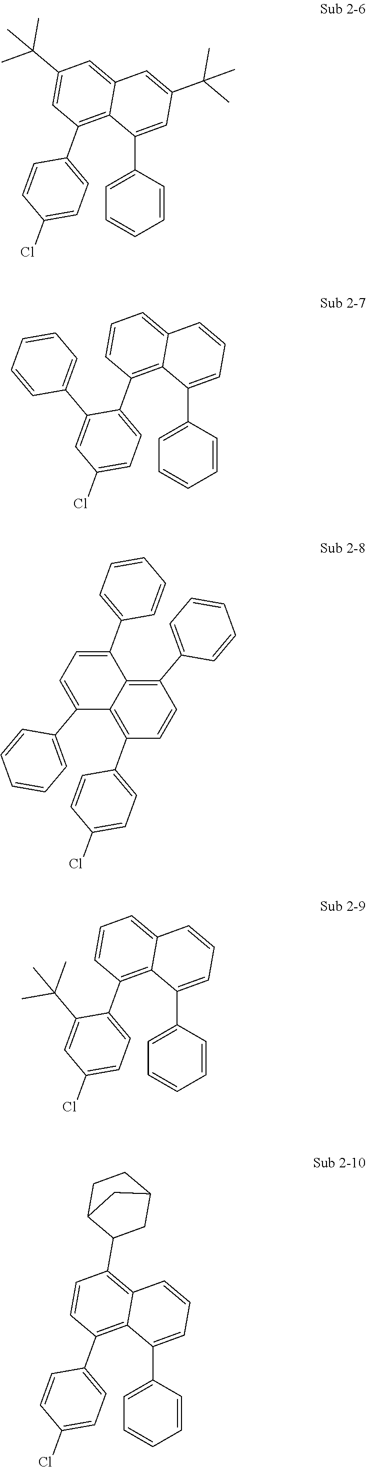

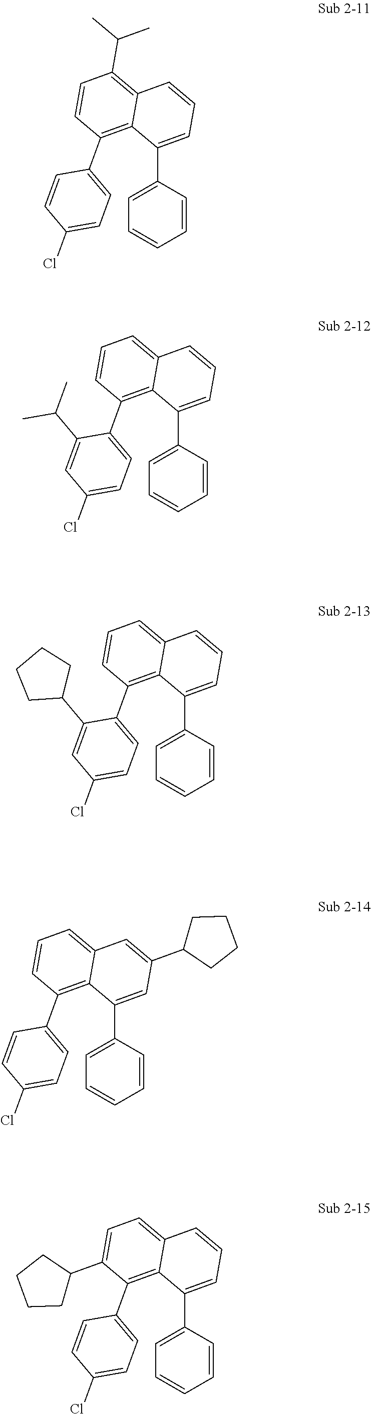 [SMILES: CC(C)c1ccc(-c2ccc(Cl)cc2)c2c(-c3ccccc3)cccc12 ; CC(C)c1cc(Cl)ccc1-c1cccc2cccc(-c3ccccc3)c12 ; Clc1ccc(-c2cccc3cccc(-c4ccccc4)c23)c(C2CCCC2)c1 ; Clc1ccc(-c2cccc3cc(C4CCCC4)cc(-c4ccccc4)c23)cc1 ; Clc1ccc(-c2c(C3CCCC3)ccc3cccc(-c4ccccc4)c23)cc1]

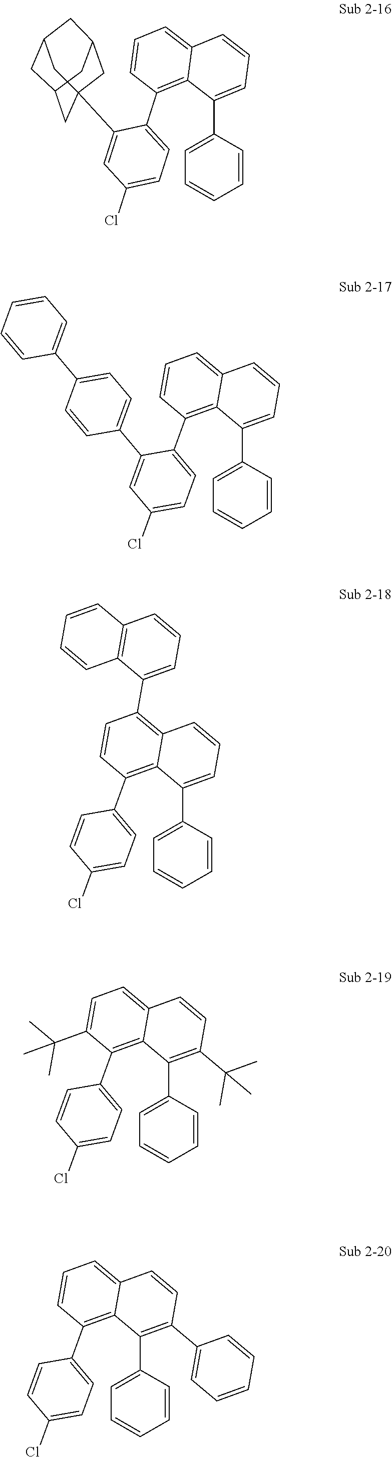 [SMILES: Clc1ccc(-c2cccc3cccc(-c4ccccc4)c23)c(C23CC4CC(CC(C4)C2)C3)c1 ; Clc1ccc(-c2cccc3cccc(-c4ccccc4)c23)c(-c2ccc(-c3ccccc3)cc2)c1 ; Clc1ccc(-c2ccc(-c3cccc4ccccc34)c3cccc(-c4ccccc4)c23)cc1 ; CC(C)(C)c1ccc2ccc(C(C)(C)C)c(-c3ccc(Cl)cc3)c2c1-c1ccccc1 ; Clc1ccc(-c2cccc3ccc(-c4ccccc4)c(-c4ccccc4)c23)cc1]

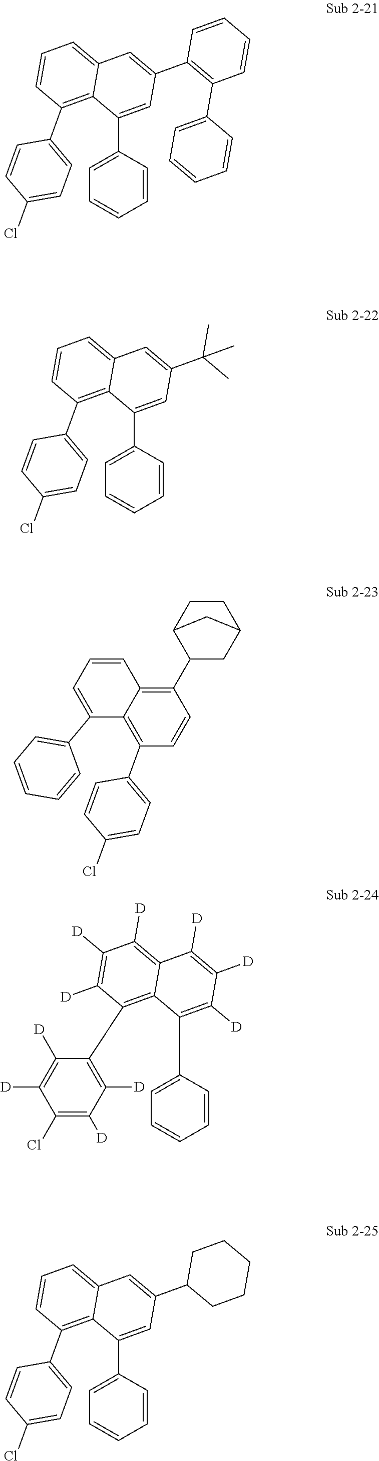 [SMILES: Clc1ccc(-c2cccc3cc(-c4ccccc4-c4ccccc4)cc(-c4ccccc4)c23)cc1 ; CC(C)(C)c1cc(-c2ccccc2)c2c(-c3ccc(Cl)cc3)cccc2c1 ; Clc1ccc(-c2ccc(C3CC4CCC3C4)c3cccc(-c4ccccc4)c23)cc1 ; [2H]c1c([2H])c(-c2c([2H])c([2H])c([2H])c3c([2H])c([2H])c([2H])c(-c4ccccc4)c23)c([2H])c([2H])c1Cl ; Clc1ccc(-c2cccc3cc(C4CCCCC4)cc(-c4ccccc4)c23)cc1]

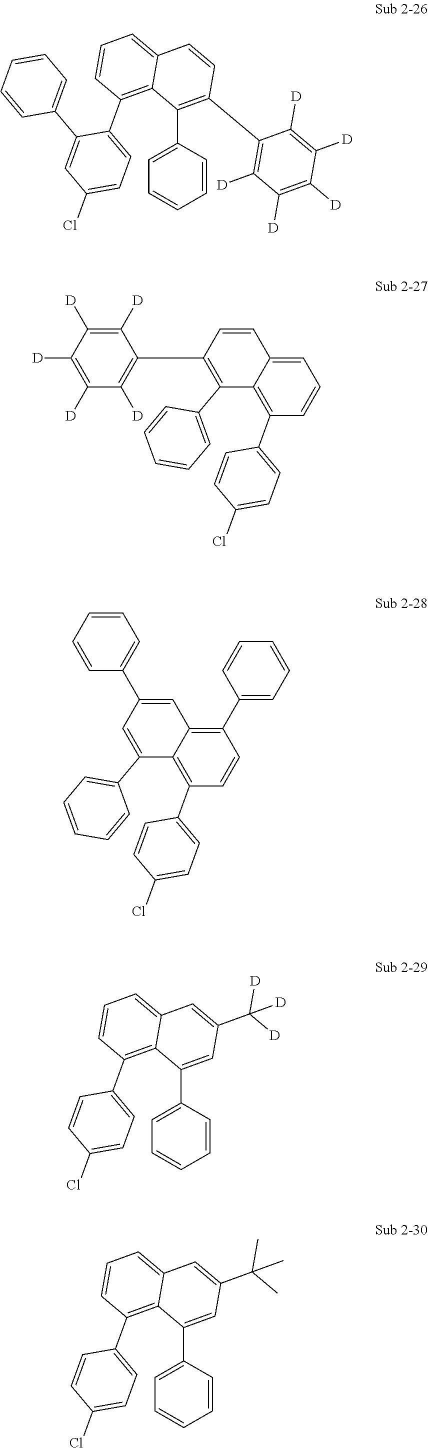 [SMILES: [2H]c1c([2H])c([2H])c(-c2ccc3cccc(-c4ccc(Cl)cc4-c4ccccc4)c3c2-c2ccccc2)c([2H])c1[2H] ; [2H]c1c([2H])c([2H])c(-c2ccc3cccc(-c4ccc(Cl)cc4)c3c2-c2ccccc2)c([2H])c1[2H] ; Clc1ccc(-c2ccc(-c3ccccc3)c3cc(-c4ccccc4)cc(-c4ccccc4)c23)cc1 ; [2H]C([2H])([2H])c1cc(-c2ccccc2)c2c(-c3ccc(Cl)cc3)cccc2c1 ; CC(C)(C)c1cc(-c2ccccc2)c2c(-c3ccc(Cl)cc3)cccc2c1]

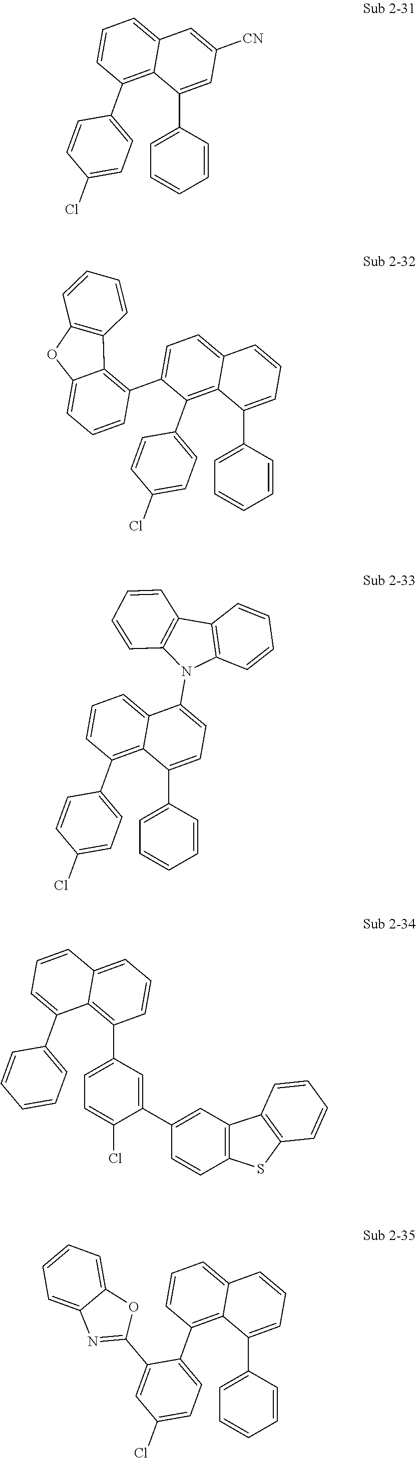 [SMILES: N#Cc1cc(-c2ccccc2)c2c(-c3ccc(Cl)cc3)cccc2c1 ; Clc1ccc(-c2c(-c3cccc4oc5ccccc5c34)ccc3cccc(-c4ccccc4)c23)cc1 ; Clc1ccc(-c2cccc3c(-n4c5ccccc5c5ccccc54)ccc(-c4ccccc4)c23)cc1 ; Clc1ccc(-c2cccc3cccc(-c4ccccc4)c23)cc1-c1ccc2sc3ccccc3c2c1 ; Clc1ccc(-c2cccc3cccc(-c4ccccc4)c23)c(-c2nc3ccccc3o2)c1]

TABLE 2

| Com- | | Com- | |
|---|---|---|---|
| pound | FD-MS | pound | FD-MS |
| Sub 2-1 | m/z = 314.09(C₂₂H₁₅Cl = 314.81) | Sub 2-2 | m/z = 390.12(C₂₈H₁₉Cl = 390.91) |
| Sub 2-3 | m/z = 390.12(C₂₈H₁₉Cl = 390.91) | Sub 2-4 | m/z = 466.15(C₃₄H₂₃Cl = 467.01) |
| Sub 2-5 | m/z = 370.15(C₂₆H₂₃Cl = 370.92) | Sub 2-6 | m/z = 426.21(C₃₀H₃₁Cl = 427.03) |
| Sub 2-7 | m/z = 390.12(C₂₈H₁₉Cl = 390.91) | Sub 2-8 | m/z = 466.15(C₃₄H₂₃Cl = 467.01) |
| Sub 2-9 | m/z = 370.15(C₂₆H₂₃Cl = 370.92) | Sub 2-10 | m/z = 408.16(C₂₉H₂₅Cl = 408.97) |
| Sub 2-11 | m/z = 356.13(C₂₅H₂₁Cl = 356.89) | Sub 2-12 | m/z = 356.13(C₂₅H₂₁Cl = 356.89) |
| Sub 2-13 | m/z = 382.15(C₂₇H₂₃Cl = 382.93) | Sub 2-14 | m/z = 382.15(C₂₇H₂₃Cl = 382.93) |
| Sub 2-15 | m/z = 382.15(C₂₇H₂₃Cl = 382.93) | Sub 2-16 | m/z = 448.2(C₃₂H₂₉Cl = 449.03) |
| Sub 2-17 | m/z = 466.15(C₃₄H₂₃Cl = 467.01) | Sub 2-18 | m/z = 440.13(C₃₂H₂₁Cl = 440.97) |
| Sub 2-19 | m/z = 426.21(C₃₀H₃₁Cl = 427.03) | Sub 2-20 | m/z = 390.12(C₂₈H₁₉Cl = 390.91) |
| Sub 2-21 | m/z = 466.15(C₃₄H₂₃Cl = 467.01) | Sub 2-22 | m/z = 370.15(C₂₆H₂₃Cl = 370.92) |
| Sub 2-23 | m/z = 408.16(C₂₉H₂₅Cl = 408.97) | Sub 2-24 | m/z = 324.15(C₂₂H₅D₁₀Cl = 324.87) |
| Sub 2-25 | m/z = 396.16(C₂₈H₂₅Cl = 396.96) | Sub 2-26 | m/z = 471.18(C₃₄H₁₈D₅Cl = 472.04) |
| Sub 2-27 | m/z = 395.15(C₂₈H₁₄D₅Cl = 395.94) | Sub 2-28 | m/z = 466.15(C₃₄H₂₃Cl = 467.01) |
| Sub 2-29 | m/z = 331.12(C₂₃H₁₄D₃Cl = 331.86) | Sub 2-30 | m/z = 370.15(C₂₆H₂₃Cl = 370.92) |
| Sub 2-31 | m/z = 339.08(C₂₃H₁₄ClN = 339.82) | Sub 2-32 | m/z = 480.13(C₃₄H₂₁ClO = 480.99) |
| Sub 2-33 | m/z = 479.14(C₃₄H₂₂ClN = 480.01) | Sub 2-34 | m/z = 496.11(C₃₄H₂₁ClS = 497.05) |
| Sub 2-35 | m/z = 431.11(C₂₉H₁₈ClNO = 431.92) | | |

### III. Synthesis of Final Product

### 1. Synthesis Example of P-1

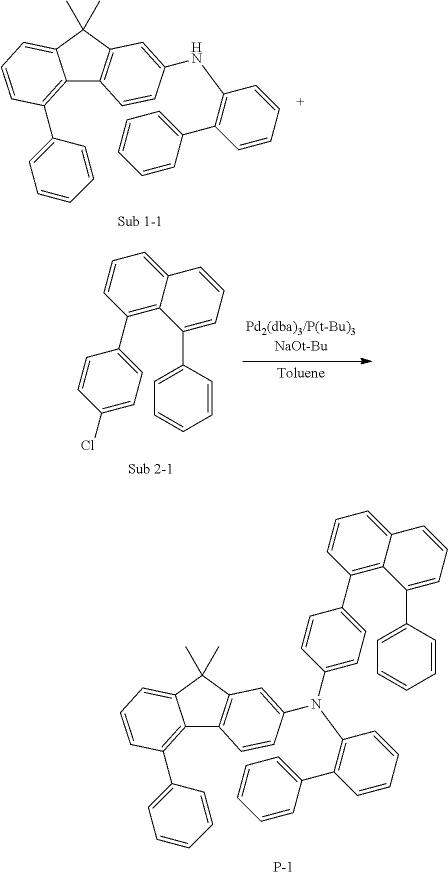

Sub 1-1 (20.0 g, 45.7 mmol) and Sub 2-1 (13.9 g, 45.7 mmol), Pd₂(dba)₃ (1.3 g, 1.4 mmol), P(t-Bu)₃ (0.6 g, 2.7 mmol), NaOt-Bu (8.8 g, 91.4 mmol), toluene (229 mL) were tested in the same manner as Sub 1-4 in a round bottom flask to obtain 23.6 g of product. (Yield: 72.1%)

### 2. Synthesis Example of P-14

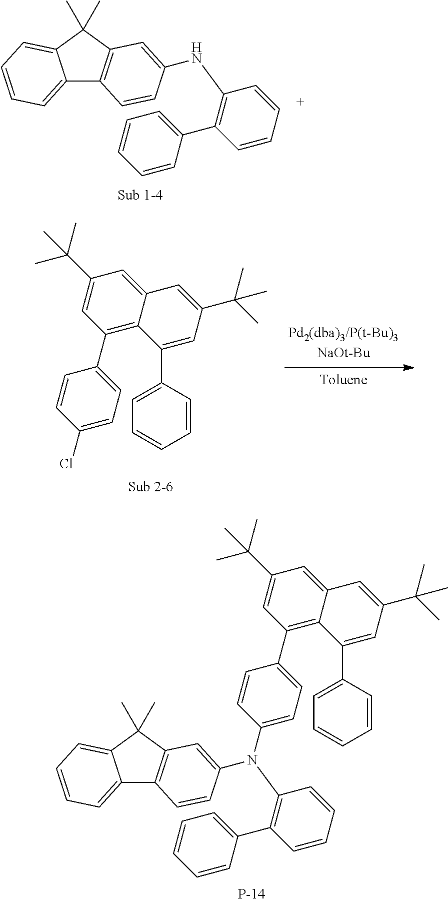

Sub 1-4 (20.0 g, 55.3 mmol) and Sub 2-6 (22.8 g, 55.3 mmol), Pd₂(dba)₃ (1.5 g, 1.7 mmol), P(t-Bu)₃ (0.7 g, 3.3 mmol), NaOt-Bu (10.6 g, 110.7 mmol), toluene (277 mL) were tested in the same manner as Sub 1-4 in a round bottom flask to obtain 28.8 g of product. (Yield: 69.3%)

### 3. Synthesis Example of P-18

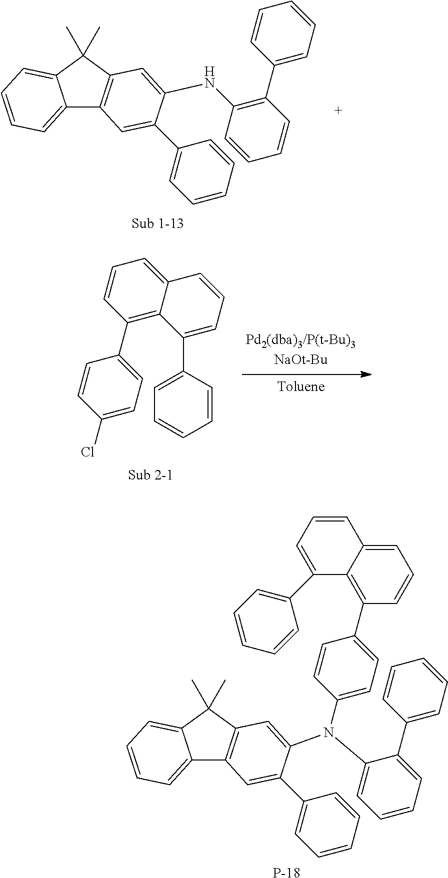

Sub 1-13 (20.0 g, 45.7 mmol) and Sub 2-1 (13.9 g, 45.7 mmol), Pd₂(dba)₃ (1.3 g, 1.4 mmol), P(t-Bu)₃ (0.6 g, 2.7 mmol), NaOt-Bu (8.8 g, 91.4 mmol), toluene (229 mL) were tested in the same manner as Sub 1-4 in a round bottom flask to obtain 22.4 g of product. (Yield: 68.6%)

### 4. Synthesis Example of P-19

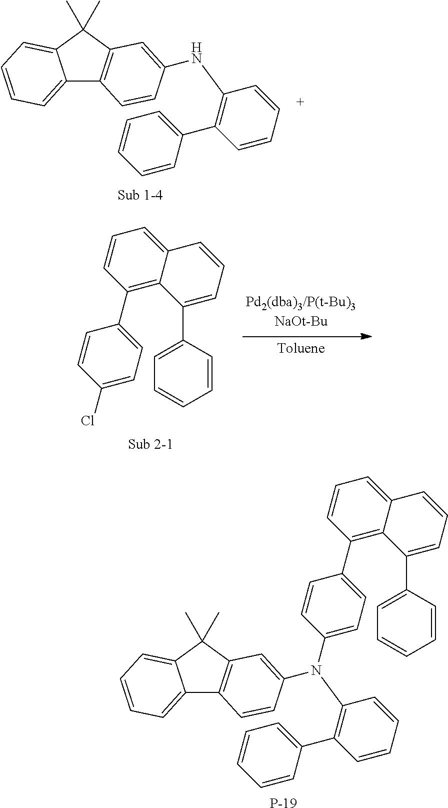

Sub 1-4 (20.0 g, 55.3 mmol) and Sub 2-1 (16.8 g, 55.3 mmol), Pd₂(dba)₃ (1.5 g, 1.7 mmol), P(t-Bu)₃ (0.7 g, 3.3 mmol), NaOt-Bu (10.6 g, 110.7 mmol), toluene (277 mL) were tested in the same manner as Sub 1-4 in a round bottom flask to obtain 26.6 g of product. (Yield: 75.1%)

### 5. Synthesis Example of P-21

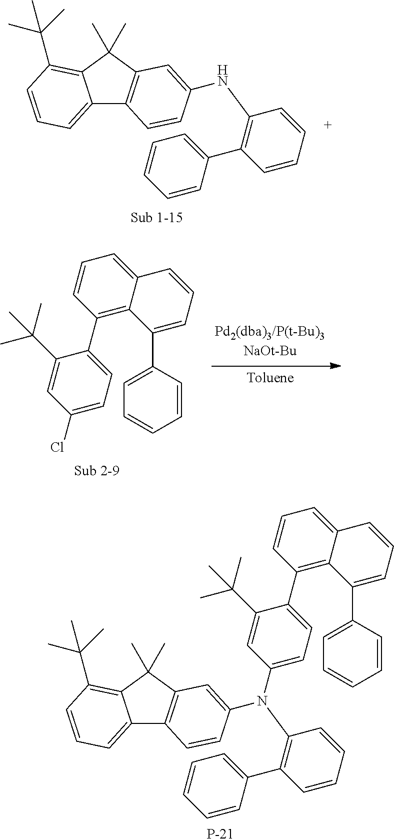

Sub 1-15 (20.0 g, 47.9 mmol) and Sub 2-9 (17.2 g, 47.9 mmol), Pd₂(dba)₃ (1.3 g, 1.4 mmol), P(t-Bu)₃ (0.5 g, 2.9 mmol), NaOt-Bu (9.2 g, 95.8 mmol), toluene (239 mL) were tested in the same manner as Sub 1-4 in a round bottom flask to obtain 25.4 g of product. (Yield: 70.4%)

### 6. Synthesis Example of P-23

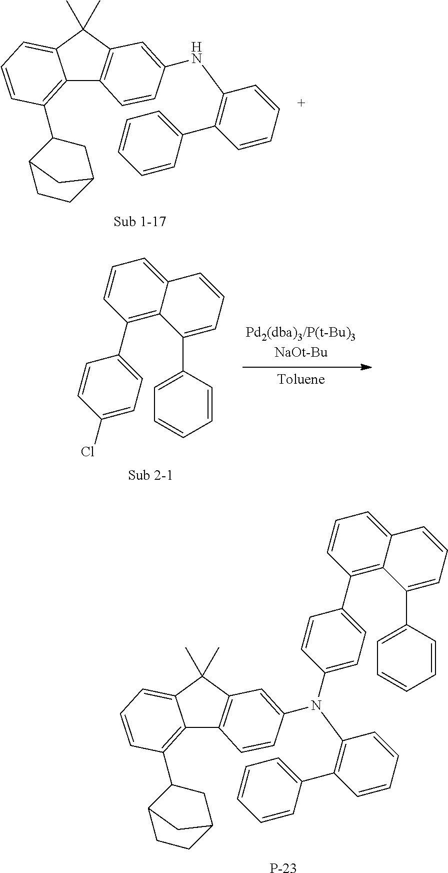

Sub 1-17 (20.0 g, 43.9 mmol) and Sub 2-1 (13.4 g, 43.9 mmol), Pd₂(dba)₃ (1.2 g, 1.3 mmol), P(t-Bu)₃ (0.5 g, 2.6 mmol), NaOt-Bu (8.4 g, 87.8 mmol), toluene (219 mL) were tested in the same manner as Sub 1-4 in a round bottom flask to obtain 23.1 g of product. (Yield: 71.6%) 7. Synthesis example of P-33

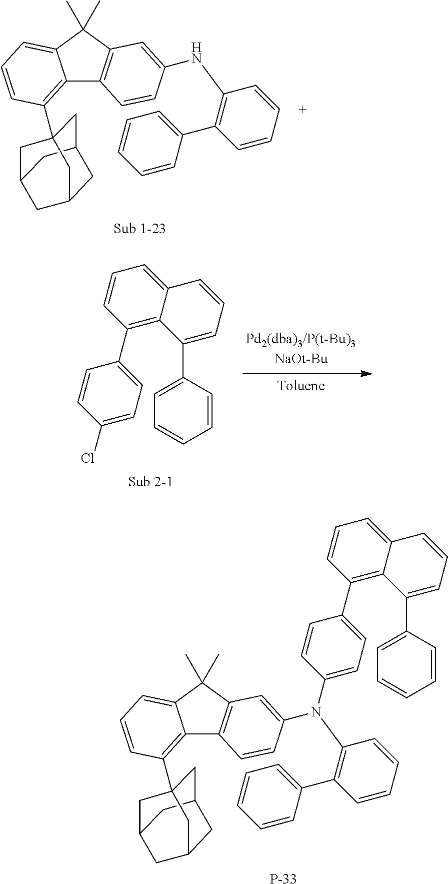

Sub 1-23 (20.0 g, 40.3 mmol) and Sub 2-1 (12.3 g, 40.3 mmol), Pd₂(dba)₃ (1.1 g, 1.2 mmol), P(t-Bu)₃ (0.5 g, 2.4 mmol), NaOt-Bu (7.8 g, 80.7 mmol), toluene (202 mL) were tested in the same manner as Sub 1-4 in a round bottom flask to obtain 22.5 g of product. (Yield: 72.1%)

### 8. Synthesis Example of P-52

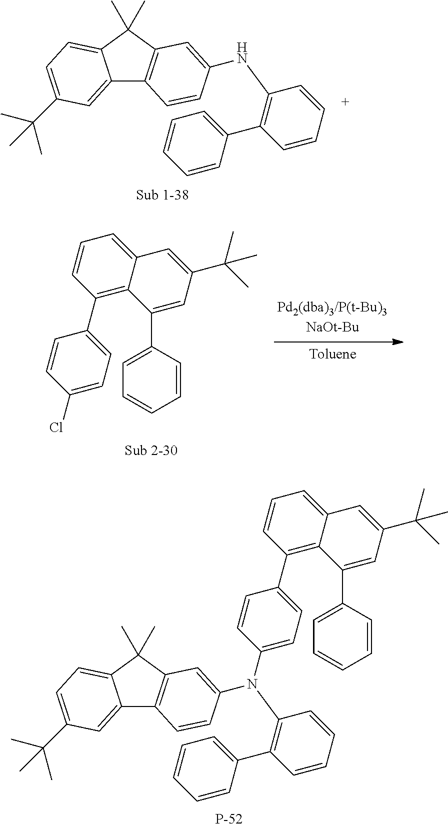

Sub 1-38 (20.0 g, 47.9 mmol) and Sub 2-30 (17.2 g, 47.9 mmol), Pd₂(dba)₃ (1.3 g, 1.4 mmol), P(t-Bu)₃ (0.6 g, 2.9 mmol), NaOt-Bu (9.2 g, 95.8 mmol), toluene (239 mL) were tested in the same manner as Sub 1-4 in a round bottom flask to obtain 25.5 g of product. (Yield: 70.7%)

### 9. Synthesis Example of P-54

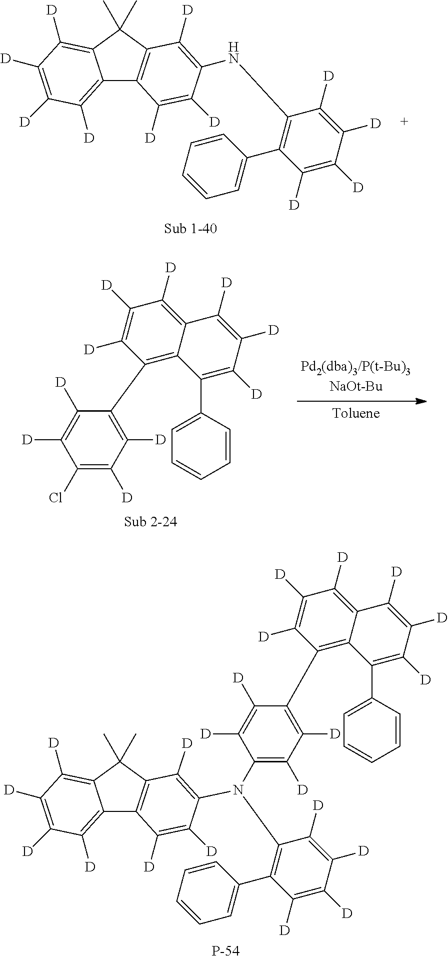

Sub 1-40 (20.0 g, 47.9 mmol) and Sub 2-24 (17.2 g, 47.9 mmol), Pd₂(dba)₃ (1.3 g, 1.4 mmol), P(t-Bu)₃ (0.6 g, 2.9 mmol), NaOt-Bu (9.2 g, 95.8 mmol), toluene (239 mL) were tested in the same manner as Sub 1-4 in a round bottom flask to obtain 25.5 g of product. (Yield: 70.7%)

### 10. Synthesis Example of P-58

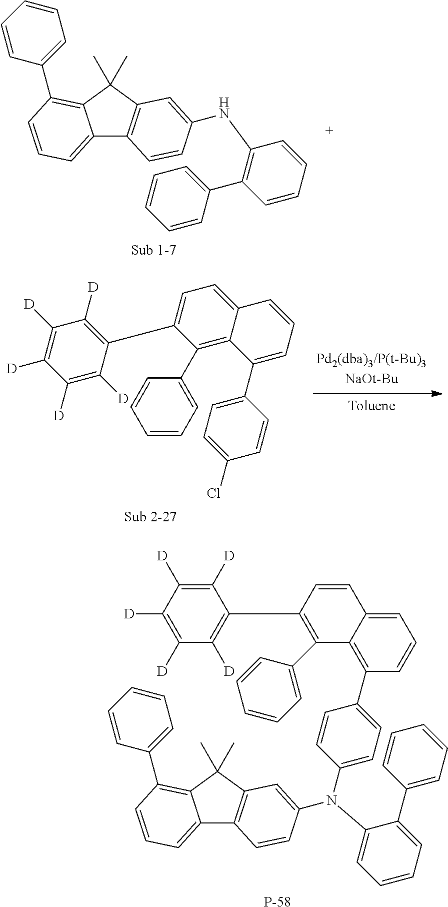

Sub 1-7 (20.0 g, 45.7 mmol) and Sub 2-27 (17.5 g, 45.7 mmol), Pd₂(dba)₃ (1.3 g, 1.4 mmol), P(t-Bu)₃ (0.6 g, 2.7 mmol), NaOt-Bu (8.8 g, 91.4 mmol), toluene (229 mL) were tested in the same manner as Sub 1-4 in a round bottom flask to obtain 25.3 g of product. (Yield: 69.4%)

### 11. Synthesis Example of P-65

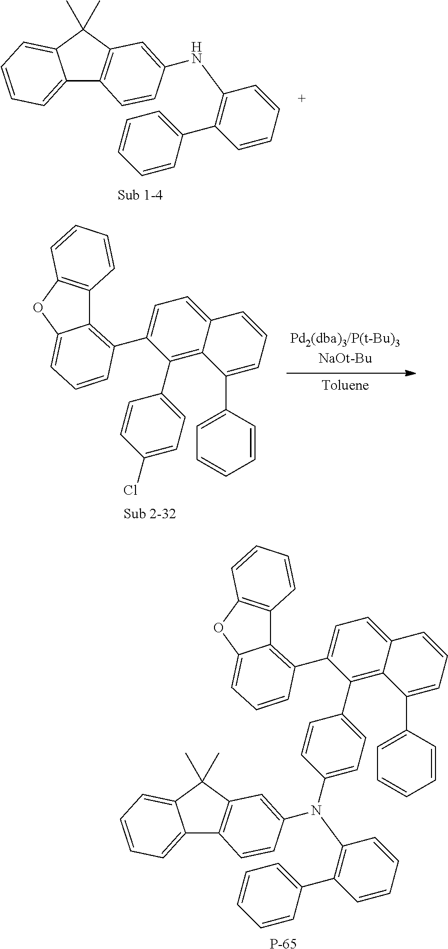

Sub 1-4 (20.0 g, 55.3 mmol) and Sub 2-32 (25.7 g, 55.3 mmol), Pd₂(dba)₃ (1.5 g, 1.7 mmol), P(t-Bu)₃ (0.7 g, 3.3 mmol), NaOt-Bu (10.6 g, 110.7 mmol), toluene (277 mL) were tested in the same manner as Sub 1-4 in a round bottom flask to obtain 32.0 g of product. (Yield: 71.7%)

### 12. Synthesis Example of P-68

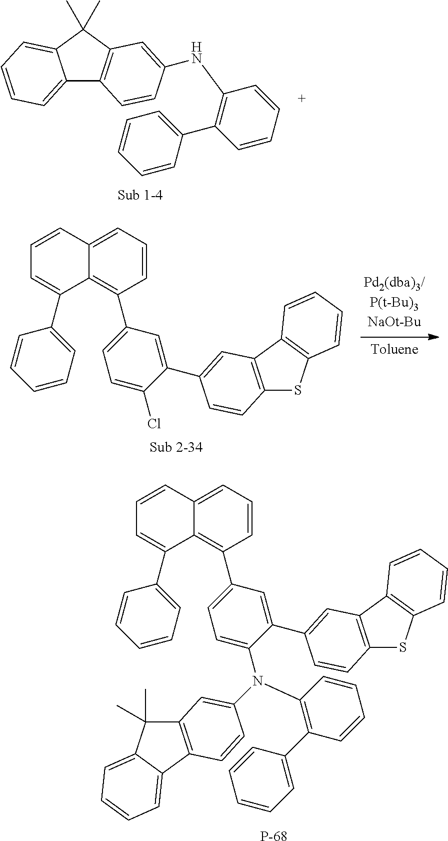

Sub 1-4 (20.0 g, 55.3 mmol) and Sub 2-34 (26.6 g, 55.3 mmol), Pd₂(dba)₃ (1.5 g, 1.7 mmol), P(t-Bu)₃ (0.7 g, 3.3 mmol), NaOt-Bu (10.6 g, 110.7 mmol), toluene (277 mL) were tested in the same manner as Sub 1-4 in a round bottom flask to obtain 31.9 g of product. (Yield: 70.2%)

### 13. Synthesis Example of P-72

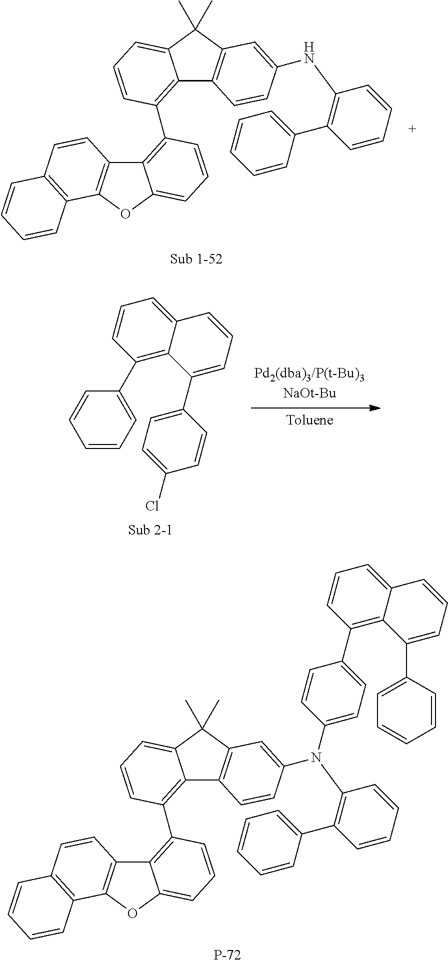

Sub 1-52 (20.0 g, 34.6 mmol) and Sub 2-1 (10.5 g, 34.6 mmol), Pd₂(dba)₃ (1.0 g, 1.0 mmol), P(t-Bu)₃ (0.4 g, 2.1 mmol), NaOt-Bu (6.7 g, 69.2 mmol), toluene (173 mL) were tested in the same manner as Sub 1-4 in a round bottom flask to obtain 20.8 g of product. (Yield: 70.1%)

### 14. Synthesis Example of P-73

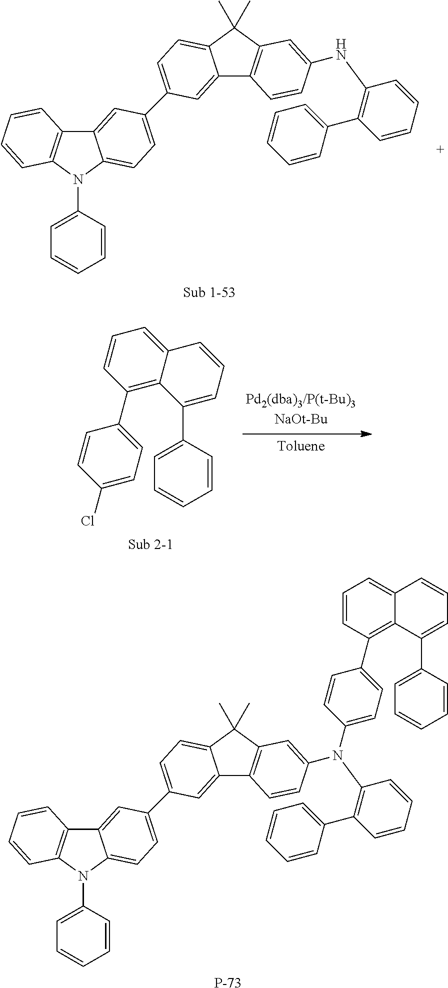

Sub 1-53 (20.0 g, 33.2 mmol) [symbol] Sub 2-1 (10.1 g, 33.2 mmol), Pd₂(dba)₃ (0.9 g, 1.0 mmol), P(t-Bu)₃ (0.4 g, 2.0 mmol), NaOt-Bu (6.4 g, 66.4 mmol), toluene (166 mL) were tested in the same manner as Sub 1-4 in a round bottom flask to obtain 19.7 g of product. (Yield: 67.5%)

Meanwhile, the FD-MS values of compounds P-1 to P-76 of the present invention prepared according to the above synthesis examples are shown in Table 3.

TABLE 3

| Com- | | Com- | |
|---|---|---|---|
| pound | FD-MS | pound | FD-MS |
| P-1 | m/z = 715.32(C₅₅H₄₁N = 715.94) | P-2 | m/z = 715.32(C₅₅H₄₁N = 715.94) |
| P-3 | m/z = 715.32(C₅₅H₄₁N = 715.94) | P-4 | m/z = 715.32(C₅₅H₄₁N = 715.94) |
| P-5 | m/z = 791.36(C₆₁H₄₅N = 792.04) | P-6 | m/z = 715.32(C₅₅H₄₁N = 715.94) |
| P-7 | m/z = 715.32(C₅₅H₄₁N = 715.94) | P-8 | m/z = 715.32(C₅₅H₄₁N = 715.94) |
| P-9 | m/z = 653.31(C₅₀H₃₉N = 653.87) | P-10 | m/z = 791.36(C₆₁H₄₅N = 792.04) |
| P-11 | m/z = 791.36(C₆₁H₄₅N = 792.04) | P-12 | m/z = 867.39(C₆₇H₄₉N = 868.14) |
| P-13 | m/z = 695.36(C₅₃H₄₅N = 695.95) | P-14 | m/z = 751.42(C₅₇H₅₃N = 752.06) |
| P-15 | m/z = 721.37(C₅₅H₄₇N = 721.99) | P-16 | m/z = 879.48(C₆₇H₆₁N = 880.23) |
| P-17 | m/z = 721.37(C₅₅H₄₇N = 721.99) | P-18 | m/z = 715.32(C₅₅H₄₁N = 715.94) |
| P-19 | m/z = 639.29(C₄₉H₃₇N = 639.84) | P-20 | m/z = 943.42(C₇₃H₅₃N = 944.23) |
| P-21 | m/z = 751.42(C₅₇H₅₃N = 752.06) | P-22 | m/z = 721.37(C₅₅H₄₇N = 721.99) |
| P-23 | m/z = 733.37(C₅₆H₄₇N = 734) | P-24 | m/z = 733.37(C₅₆H₄₇N = 734) |
| P-25 | m/z = 667.32(C₅₁H₄₁N = 667.9) | P-26 | m/z = 667.32(C₅₁H₄₁N = 667.9) |
| P-27 | m/z = 681.34(C₅₂H₄₃N = 681.92) | P-28 | m/z = 723.39(C₅₅H₄₉N = 724) |
| P-29 | m/z = 707.36(C₅₄H₄₅N = 707.96) | P-30 | m/z = 707.36(C₅₄H₄₅N = 707.96) |
| P-31 | m/z = 707.36(C₅₄H₄₅N = 707.96) | P-32 | m/z = 775.42(C₅₉H₅₃N = 776.08) |
| P-33 | m/z = 773.4(C₅₉H₅₁N = 774.06) | P-34 | m/z = 773.4(C₅₉H₅₁N = 774.06) |
| P-35 | m/z = 719.35(C₅₅H₃₇D₄N = 719.96) | P-36 | m/z = 795.38(C₆₁H₄₁D₄N = 796.06) |
| P-37 | m/z = 867.39(C₆₇H₄₉N = 868.14) | P-38 | m/z = 796.39(C₆₁H₄₀D₅N = 797.07) |
| P-39 | m/z = 765.34(C₅₉H₄₃N = 766) | P-40 | m/z = 765.34(C₅₉H₄₃N = 766) |
| P-41 | m/z = 765.34(C₅₉H₄₃N = 766) | P-42 | m/z = 917.4(C₇₁H₅₁N = 918.2) |
| P-43 | m/z = 841.37(C₆₅H₄₇N = 842.1) | P-44 | m/z = 765.34(C₅₉H₄₃N = 766) |
| P-45 | m/z = 791.36(C₆₁H₄₅N = 792.04) | P-46 | m/z = 791.36(C₆₁H₄₅N = 792.04) |
| P-47 | m/z = 891.39(C₆₉H₄₉N = 892.16) | P-48 | m/z = 791.36(C₆₁H₄₅N = 792.04) |
| P-49 | m/z = 751.42(C₅₇H₅₃N = 752.06) | P-50 | m/z = 715.32(C₅₅H₄₁N = 715.94) |
| P-51 | m/z = 791.36(C₆₁H₄₅N = 792.04) | P-52 | m/z = 751.42(C₅₇H₅₃N = 752.06) |
| P-53 | m/z = 740.41(C₅₆H₄₀D₇N = 741.04) | P-54 | m/z = 660.42(C₄₉H₁₆D₂₁N = 660.97) |
| P-55 | m/z = 802.43(C₆₁H₄₆D₅N = 803.12) | P-56 | m/z = 796.39(C₆₁H₄₀D₅N = 797.07) |
| P-57 | m/z = 719.35(C₅₅H₃₇D₄N = 719.96) | P-58 | m/z = 796.39(C₆₁H₄₀D₅N = 797.07) |
| P-59 | m/z = 723.39(C₅₅H₄₉N = 724) | P-60 | m/z = 798.4(C₆₁H₃₈D₇N = 799.08) |
| P-61 | m/z = 673.36(C₅₁H₃₅D₆N = 673.93) | P-62 | m/z = 821.37(C₆₂H₄₇NO = 822.06) |
| P-63 | m/z = 797.4(C₆₁H₅₁N = 798.09) | P-64 | m/z = 746.37(C₅₆H₄₆N₂ = 747) |
| P-65 | m/z = 805.33(C₆₁H₄₃NO = 806.02) | P-66 | m/z = 908.41(C₆₇H₃₆D₁₁NS = 909.25) |
| P-67 | m/z = 804.35(C₆₁H₄₄N₂ = 805.04) | P-68 | m/z = 821.31(C₆₁H₄₃NS = 822.08) |
| P-69 | m/z = 831.39(C₆₄H₄₉N = 832.1) | P-70 | m/z = 815.36(C₆₃H₄₅N = 816.06) |
| P-71 | m/z = 880.38(C₆₇H₄₈N₂ = 881.13) | P-72 | m/z = 855.35(C₆₅H₄₅NO = 856.08) |
| P-73 | m/z = 880.38(C₆₇H₄₈N₂ = 881.13) | P-74 | m/z = 716.32(C₅₄H₄₀N₂ = 716.93) |
| P-75 | m/z = 880.38(C₆₇H₄₈N₂ = 881.13) | P-76 | m/z = 756.31(C₅₆H₄₀N₂O = 756.95) |

### Manufacturing Evaluation of Organic Electronic Elevements

### [Example 1] Green Organic Light Emitting Device (Hole Transport Layer)

N¹-(naphthalen-2-yl)-N⁴,N⁴-bis(4-(naphthalen-2-yl(phenyl)amino)phenyl)-N¹-phenylbenzene-1,4-diamine (hereinafter abbreviated as 2-TNATA) was vacuum deposited on the ITO layer (anode) formed on the glass substrate to form a hole injection layer with a thickness of 60 nm. On the hole injection layer, compound P-1 of the present invention represented by Formula 1 was vacuum deposited to a thickness of 60 nm to form a hole transport layer.

Subsequently, 4,4′-N,N′-dicarbazole-biphenyl (hereinafter abbreviated as CPB) was used as the host material of the emitting layer and Ir(ppy)₃ [tris(2-phenylpyridine)-iridium] was used as the dopant material on the hole transport layer, and the dopants were doped in a 90:10 weight ratio to form an emitting layer with a thickness of 30 nm. Next, (1, 1′-biphenyl-4-olato)bis(2-methyl-8-quinolinolato)aluminum (hereinafter abbreviated as BAlq) is vacuum deposited on the emitting layer to form a hole blocking layer with a thickness of 10 nm, and bis(10-hydroxybenzo[h]quinolinato)beryllium (hereinafter abbreviated as BeBq₂) was vacuum deposited to a thickness of 40 nm on the hole blocking layer to form an electron transport layer. Afterwards, LiF was deposited on the electron transport layer to form an electron injection layer with a thickness of 0.2 nm, and then Al was deposited to form a cathode with a thickness of 150 nm.

### [Example 2] to [Example 25]

An organic light emitting device was manufactured in the same manner as in Example 1, except that the compound of the present invention shown in Table 4 was used as the hole transport layer material instead of Compound P-1 of the present invention.

### [Comparative Example 1] and [Comparative Example 4]

An organic light emitting device was manufactured in the same manner as in Example 1, except that Comparative compound A or Comparative compound B was used as the hole transport layer material instead of Compound P-1 of the present invention.

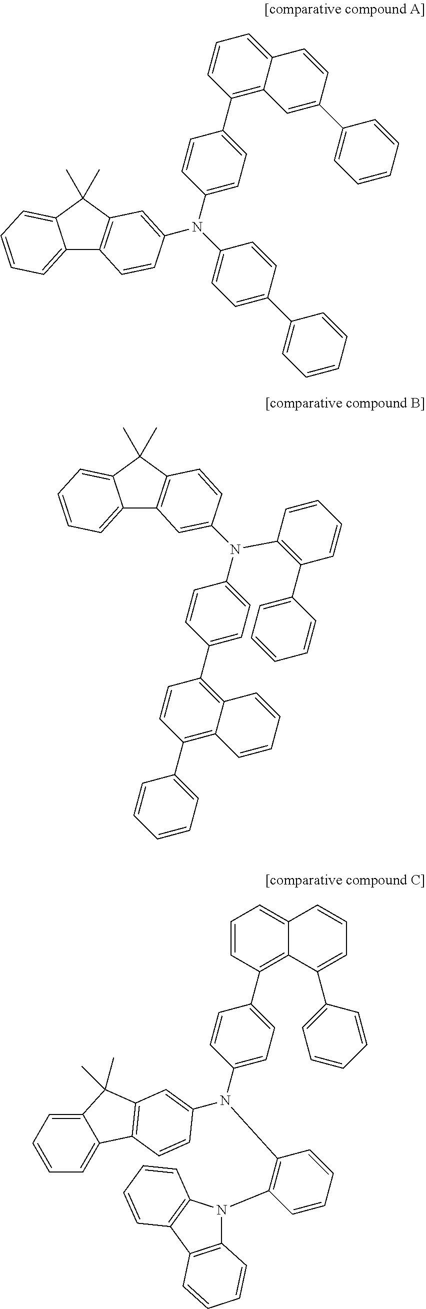 [SMILES: CC1(C)c2ccccc2-c2ccc(N(c3ccc(-c4ccccc4)cc3)c3ccc(-c4cccc5ccc(-c6ccccc6)cc45)cc3)cc21 ; CC1(C)c2ccccc2-c2cc(N(c3ccc(-c4ccc(-c5ccccc5)c5ccccc45)cc3)c3ccccc3-c3ccccc3)ccc21 ; CC1(C)c2ccccc2-c2ccc(N(c3ccc(-c4cccc5cccc(-c6ccccc6)c45)cc3)c3ccccc3-n3c4ccccc4c4ccccc43)cc21]

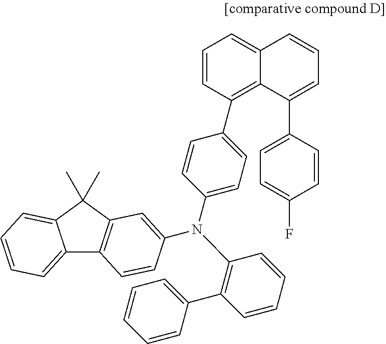 [SMILES: CC1(C)c2ccccc2-c2ccc(N(c3ccc(-c4cccc5cccc(-c6ccc(F)cc6)c45)cc3)c3ccccc3-c3ccccc3)cc21]

To the organic electroluminescent device manufactured by Examples 1 to 25 and Comparative Examples 1 to 4 of the present invention, Electroluminescence (EL) characteristics were measured with a PR-650 of Photoresearch Co., by applying a forward bias DC voltage. As a result of the measurement, T95 life was measured at a standard luminance of 5,000 cd/m² through life measuring apparatus manufactured by McScience. Table 4 shows the results of device fabrication and evaluation.

TABLE 4

| | | | Current | | | |
|---|---|---|---|---|---|---|
| | | | Density | Brightness | Efficiency | |
| | Compound | Voltage | (mA/cm²) | (cd/m²) | (cd/A) | T(95) |
| Comparative | Comparative | 5.8 | 21.1 | 5000.0 | 23.7 | 81.7 |
| example (1) | compound A | | | | | |
| Comparative | Comparative | 5.8 | 19.5 | 5000.0 | 25.6 | 82.2 |
| example (2) | compound B | | | | | |
| Comparative | Comparative | 6.1 | 18.8 | 5000.0 | 26.6 | 83.8 |
| example (3) | compound C | | | | | |
| Comparative | Comparative | 5.9 | 20.2 | 5000.0 | 24.8 | 84.3 |
| example (4) | compound D | | | | | |
| Example (1) | P-1 | 5.4 | 12.0 | 5000.0 | 41.8 | 109.6 |
| Example (2) | P-3 | 5.5 | 12.8 | 5000.0 | 39.0 | 106.6 |
| Example (3) | P-6 | 5.3 | 12.5 | 5000.0 | 39.9 | 107.2 |
| Example (4) | P-13 | 5.3 | 12.3 | 5000.0 | 40.5 | 105.0 |
| Example (5) | P-14 | 5.5 | 12.6 | 5000.0 | 39.6 | 104.3 |
| Example (6) | P-15 | 5.5 | 12.6 | 5000.0 | 39.7 | 103.8 |
| Example (7) | P-16 | 5.5 | 12.8 | 5000.0 | 39.2 | 103.7 |
| Example (8) | P-18 | 5.4 | 12.1 | 5000.0 | 41.3 | 108.9 |
| Example (9) | P-19 | 5.2 | 11.5 | 5000.0 | 43.3 | 110.1 |
| Example (10) | P-21 | 5.5 | 12.8 | 5000.0 | 39.1 | 103.2 |
| Example (11) | P-23 | 5.5 | 12.8 | 5000.0 | 39.1 | 103.2 |
| Example (12) | P-33 | 5.5 | 12.8 | 5000.0 | 39.2 | 102.8 |
| Example (13) | P-34 | 5.3 | 12.3 | 5000.0 | 40.7 | 105.9 |
| Example (14) | P-38 | 5.4 | 12.6 | 5000.0 | 39.8 | 107.6 |
| Example (15) | P-44 | 5.3 | 12.4 | 5000.0 | 40.3 | 108.4 |
| Example (16) | P-50 | 5.3 | 12.7 | 5000.0 | 39.4 | 106.5 |
| Example (17) | P-52 | 5.4 | 12.8 | 5000.0 | 39.1 | 104.8 |
| Example (18) | P-54 | 5.2 | 11.3 | 5000.0 | 44.2 | 111.3 |
| Example (19) | P-58 | 5.5 | 13.0 | 5000.0 | 38.5 | 105.4 |
| Example (20) | P-59 | 5.5 | 13.0 | 5000.0 | 38.6 | 103.2 |
| Example (21) | P-65 | 5.4 | 13.2 | 5000.0 | 37.9 | 100.3 |
| Example (22) | P-66 | 5.5 | 13.3 | 5000.0 | 37.7 | 102.1 |
| Example (23) | P-68 | 5.4 | 13.2 | 5000.0 | 38.0 | 101.7 |
| Example (24) | P-72 | 5.5 | 13.6 | 5000.0 | 36.9 | 100.4 |
| Example (25) | P-73 | 5.5 | 13.6 | 5000.0 | 36.8 | 100.5 |

As can be seen from the results in Table 4, when a green organic electroluminescent device is manufactured using the material for an organic electroluminescent device of the present invention as a hole transport layer material, the driving voltage, luminous efficiency, and lifespan of the organic electroluminescent device can be improved compared to the comparative examples using Comparative Compounds A to Comparative Compounds D, which have similar basic structures to the compound of the present invention. In order to interpret the results in Table 4, the composition of the compound represented by Formula 1 of the present invention will be described as Component 1, Component 2, and Component 3.

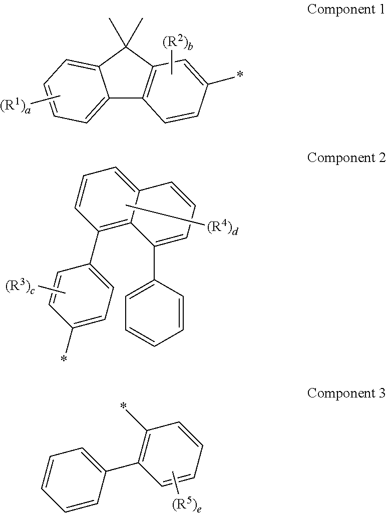

Comparative compound A is a tertiary amine compound comprising the group represented by Component 1 in the molecule, but when compared to the compound of the present invention, it is different from the compound of the present invention in that the moiety corresponding to Component 2 is 4-(7-phenylnaphthalen-1-yl)phenyl, and the moiety corresponding to Component 3 is 1,1′-biphenyl-4-yl.

Comparative compound B is a tertiary amine compound comprising the group represented by Component 3 in the molecule, but when compared to the compound of the present invention, it is different from the compound of the present invention in that the group corresponding to Component 1 is 9,9-dimethylfluorene, which is bonded to an amine group at position 3, and the moiety corresponding to Component 2 is 4-(4-phenylnaphthalen-1-yl)phenyl group.

That is, when compared to compound P-19 of the present invention, Comparative Compound A and Comparative Compound B are isomers with the same molecular formula, but the substitution positions of the groups corresponding to Component 1, Component 2, and Component 3 are different compared with the compound of the present invention.

Due to this difference, the compound of the present invention has greater steric hindrance than Comparative Compound A and Comparative Compound B, which has the effect of lowering the crystallinity of the thin film, that is, creating an amorphous state. Therefore, when applying the compound of the present invention to a device, the stability of the compound itself is higher than that of Comparative Compound A and Comparative Compound B, and the hole mobility is also excellent, improving the charge balance of the entire device, and although the planarity of the molecule is poor, the Tg value is reduced, so the device can be manufactured even at a relatively low temperature during deposition, and the device results are judged to be significantly superior.

Comparative compound C is similar to the compound of the present invention in that it is a tertiary amine compound comprising groups represented by Component 1 and Component 2 in the molecule, but Comparative compound C is different from the compound of the present invention in that the moiety corresponding to Component 3 is a 2-(9H-carbazol-9-yl)phenyl group.

Comparative compound D is similar to the compound of the present invention in that it is a tertiary amine comprising groups represented by Component 1 and Component 3 in the molecule, but Comparative compound D is different from the compound of the present invention in that Component 2, which is an essential component of the present invention, is further substituted with fluorine.

The substitution positions of the compounds are similar to those of Comparative Compound C and Comparative Compound D, but in order to confirm the difference in the energy level of the compound resulting from the difference in the type of substituent, the data measured using the DFT method (B3LYP/6-31g(D)) of the Gaussian program are shown in Table 5.

TABLE 5

| | Comparative | Comparative | |
|---|---|---|---|
| | compound C | compound D | P-19 |
| HOMO | −4.845 | −4.756 | −4.738 |
| (eV) | | | |
| LUMO | −1.208 | −1.265 | −1.203 |
| (eV) | | | |

As can be seen from Table 5, it can be confirmed that there is an energy level difference between the compound of the present invention and the comparative compounds due to some differences in the composition of the compounds. First, when comparing the compound of the present invention with comparative compound C, which has a large difference in HOMO level, since the compound of the present invention has a smaller energy level difference with ITO (anode) than comparative compound C, it is believed to have excellent hole properties, that is, hole injection properties and hole transport properties, and thus have an excellent effect on the device results.

Also, Comparative Compound D is judged to have a lower LUMO level than the compound of the present invention because F (fluorine), which has electron-attracting properties, is substituted within the molecule. Therefore, it is believed that the compound of the present invention, which has a higher LUMO level than Comparative Compound D, has an excellent effect on the device results because it can effectively block electrons coming from the emitting layer.

That is, because the compound of the present invention has a more appropriate energy level as a hole transport layer than Comparative Compound C and Comparative Compound D, it is believed that the charge balance of the entire device is improved and the device results are remarkable.

That is, the compound of the present invention, which satisfies all the relevant compositions, showed significant effects in terms drive, efficiency, and lifespan of device data compared to comparative compounds, even if the basic molecular structure is similar to Comparative Compound A to Comparative Compound D and the compound of the present invention, depending on the position of the substituent and the type of the substituent, the properties of compounds, such as hole characteristics, light efficiency characteristics, energy level, hole injection and mobility characteristics, charge balance between holes and electrons, volume density, and intermolecular distance, can vary significantly enough to be difficult to predict, and additionally, it suggests that one configuration does not affect the results of the entire device, but that the performance of the device can vary due to complex factors.

In addition, in the evaluation results of the above-described device fabrication, the device characteristics were explained by applying the compound of the present invention only to the hole transport layer, but the compound of the present invention can be used by applying it to an emitting-auxiliary layer or by applying it to both a hole transport layer and an emitting-auxiliary layer. However, it is explained through the device results that the preferred layer for use of the compound of the present invention is the hole transport layer.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device with excellent device characteristics such as high brightness, high luminescence, and long lifespan, and thus has industrial applicability.

What is claimed is:

1. A compound represented by Formula 1: 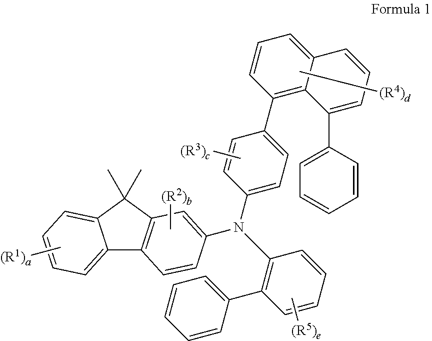
wherein:
1) R¹, R², R³, R⁴ and R⁵ are each independently selected from the group consisting of hydrogen; deuterium; a cyano group: a C₆-C₆₀ aryl group; a fluorenyl group; a C₂-C₆₀ heteroaryl group including at least one heteroatom of O, N, S, Si and P; a C₃-C₆₀ cycloalkyl group; a C₁-C₆₀ alkyl group; a C₂-C₂₀ alkenyl group; a C₂-C₂₀ alkynyl group; a C₁-C₃₀ alkoxyl group; and a C₆-C₃₀ aryloxy group, wherein adjacent groups are not bonded to each other to form a ring,
2) a, c and e are independently an integer of 0 to 4, b is an integer of 0 to 3, d is an integer of 0 to 6,
3) wherein the aryl group, heteroaryl group, fluorenyl group, cycloalkyl group, alkyl group, alkenyl group, alkynyl group, alkoxyl group and aryloxy group may be substituted with one or more substituents selected from the group consisting of deuterium; a cyano group; a nitro group; a C₁-C₂₀ alkylthio group; a C₁-C₂₀ alkoxyl group; a C₆-C₂₀ aryloxy group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ cycloalkyl group; a C₇-C₂₀ arylalkyl group; and a C₈-C₂₀ arylalkenyl group, wherein the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The compound of claim 1, wherein any one of R¹, R², R³, R⁴ and R⁵ is selected from the group consisting of a C₁-C₁₀ alkyl group; a C₃-C₁₀ cycloalkyl group; and a C₆-C₁₈ aryl group.

3. The compound of claim 1, wherein any one of R¹, R², R³, R⁴ and R⁵ is selected from the group consisting of hydrogen; deuterium; and a substituent represented by any of Formulas R-1 to R-13: 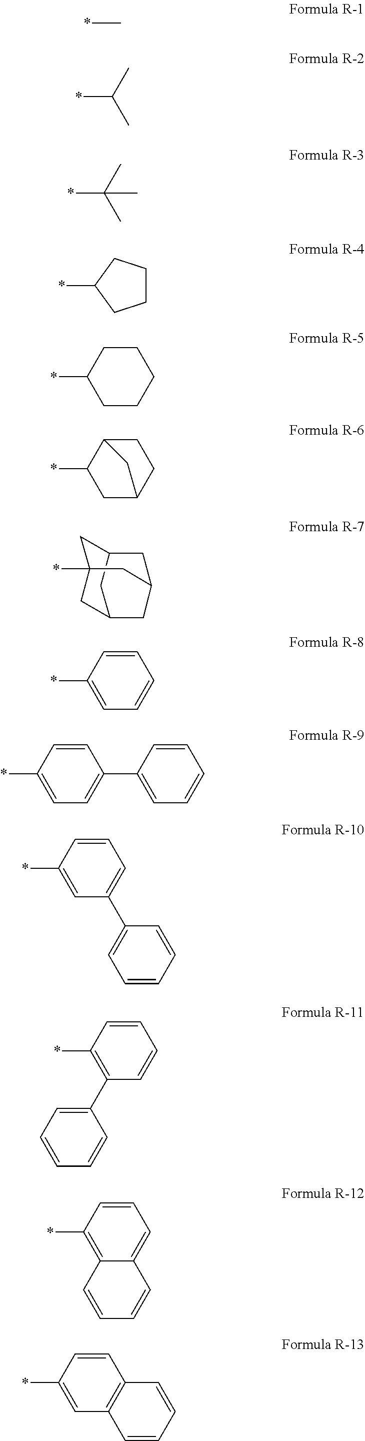
wherein * indicates the position to be bonded.

4. The compound of claim 1, wherein R¹, R², R³, R⁴ and R⁵ are hydrogen or deuterium.

5. The compound of claim 1, wherein Formula 1 is represented by any Formulas 1-1 to 1-9: 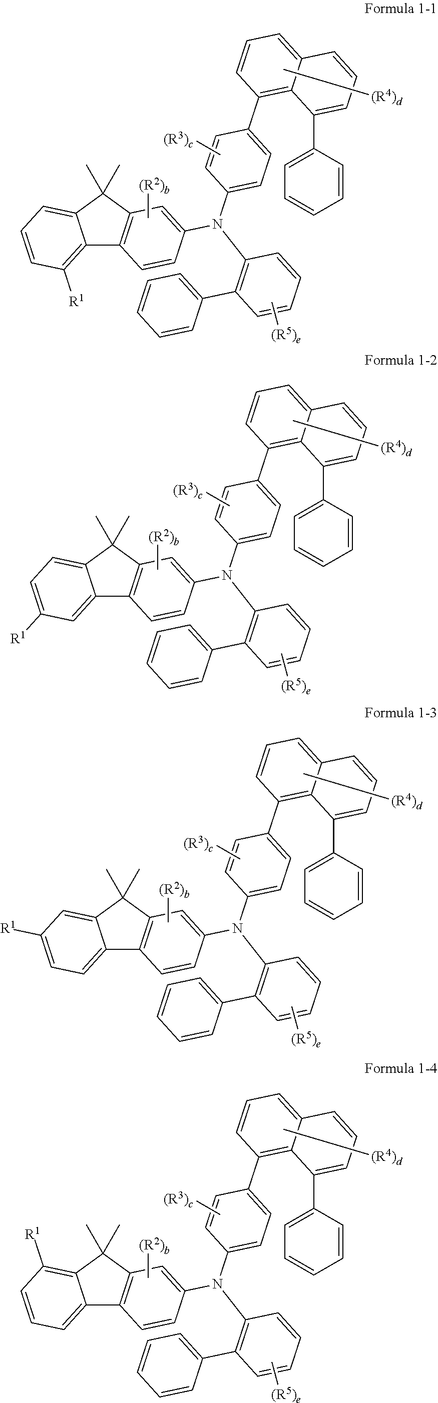 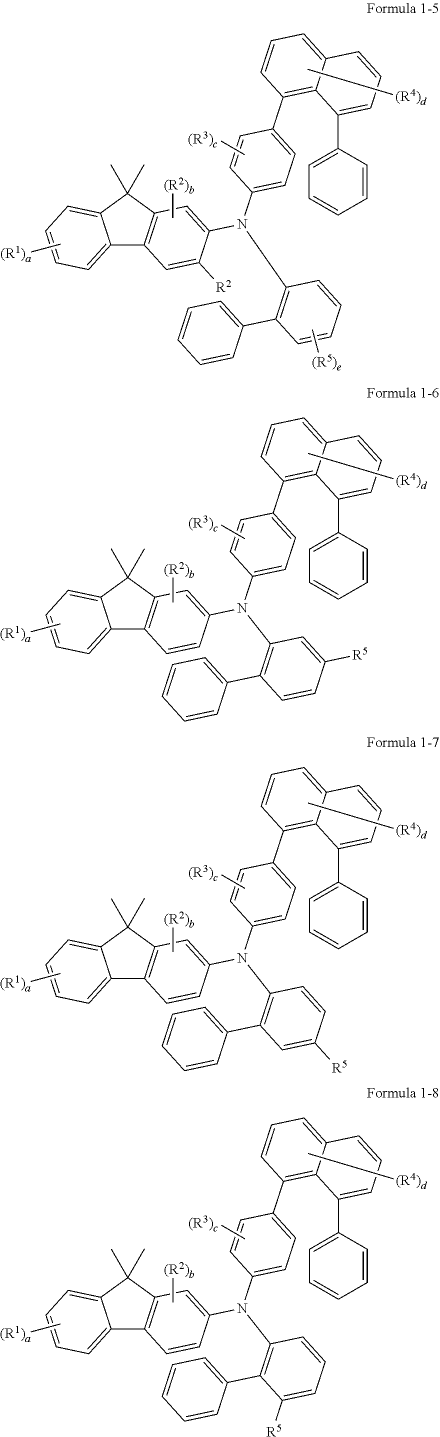 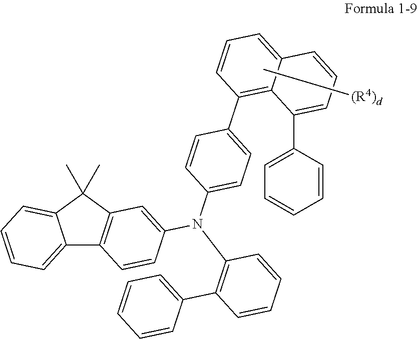
wherein: R¹, R², R³, R⁴, R⁵, a, b, c, d and e are the same as defined in claim 1.

6. The compound of claim 1, wherein Formula 1 is represented by any of the following compounds P-1 to P-76:

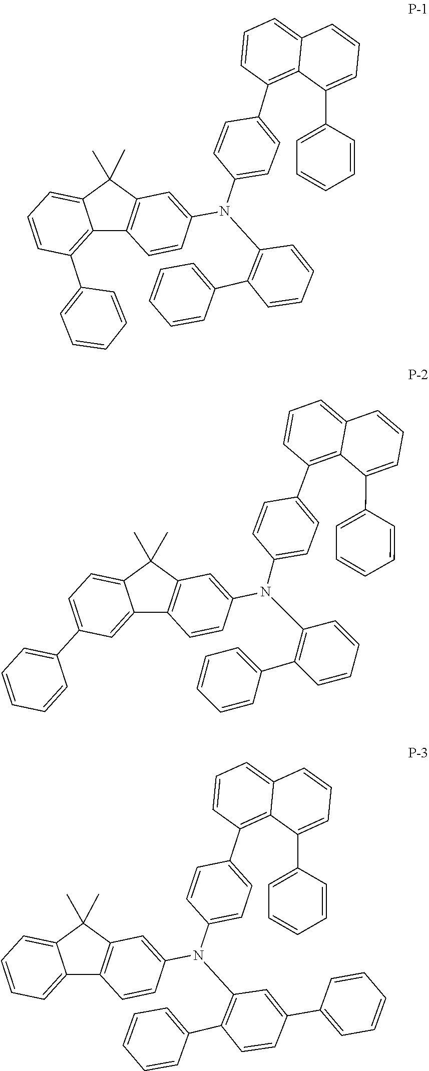 [SMILES: CC1(C)c2cc(N(c3ccc(-c4cccc5cccc(-c6ccccc6)c45)cc3)c3ccccc3-c3ccccc3)ccc2-c2c(-c3ccccc3)cccc21 ; CC1(C)c2ccc(-c3ccccc3)cc2-c2ccc(N(c3ccc(-c4cccc5cccc(-c6ccccc6)c45)cc3)c3ccccc3-c3ccccc3)cc21 ; CC1(C)c2ccccc2-c2ccc(N(c3ccc(-c4cccc5cccc(-c6ccccc6)c45)cc3)c3cc(-c4ccccc4)ccc3-c3ccccc3)cc21]

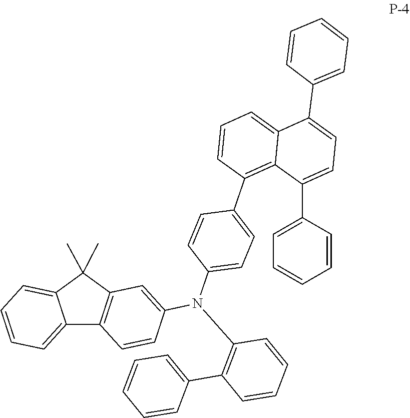 [SMILES: CC1(C)c2ccccc2-c2ccc(N(c3ccc(-c4cccc5c(-c6ccccc6)ccc(-c6ccccc6)c45)cc3)c3ccccc3-c3ccccc3)cc21]

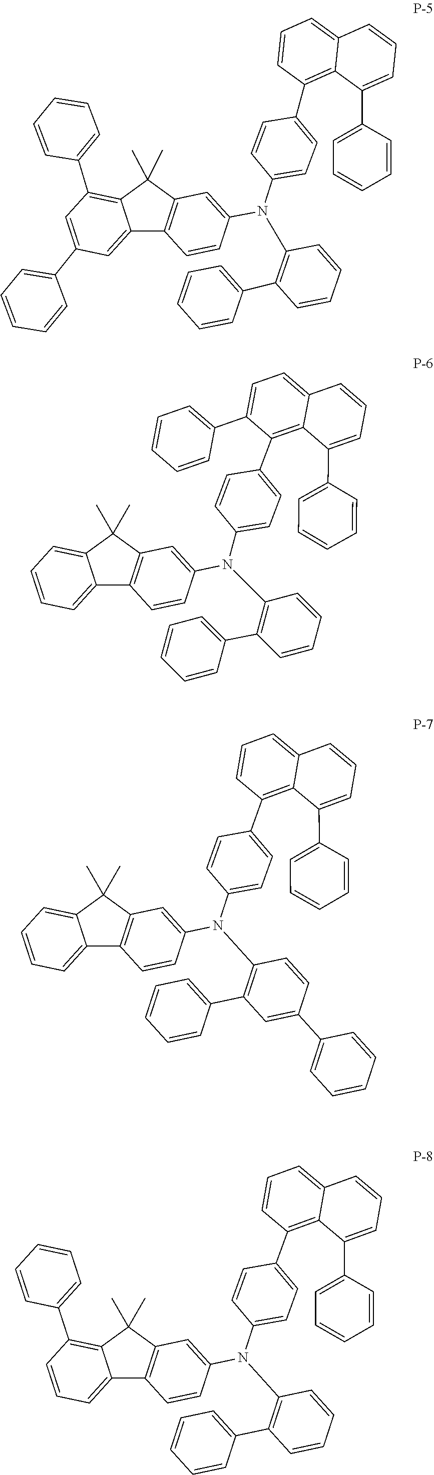 [SMILES: CC1(C)c2cc(N(c3ccc(-c4cccc5cccc(-c6ccccc6)c45)cc3)c3ccccc3-c3ccccc3)ccc2-c2cc(-c3ccccc3)cc(-c3ccccc3)c21 ; CC1(C)c2ccccc2-c2ccc(N(c3ccc(-c4c(-c5ccccc5)ccc5cccc(-c6ccccc6)c45)cc3)c3ccccc3-c3ccccc3)cc21 ; CC1(C)c2ccccc2-c2ccc(N(c3ccc(-c4cccc5cccc(-c6ccccc6)c45)cc3)c3ccc(-c4ccccc4)cc3-c3ccccc3)cc21 ; CC1(C)c2cc(N(c3ccc(-c4cccc5cccc(-c6ccccc6)c45)cc3)c3ccccc3-c3ccccc3)ccc2-c2cccc(-c3ccccc3)c21]

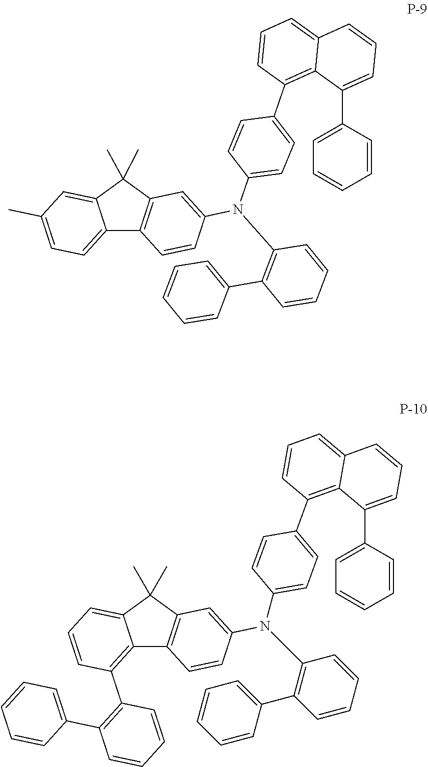 [SMILES: Cc1ccc2c(c1)C(C)(C)c1cc(N(c3ccc(-c4cccc5cccc(-c6ccccc6)c45)cc3)c3ccccc3-c3ccccc3)ccc1-2 ; CC1(C)c2cc(N(c3ccc(-c4cccc5cccc(-c6ccccc6)c45)cc3)c3ccccc3-c3ccccc3)ccc2-c2c(-c3ccccc3-c3ccccc3)cccc21]

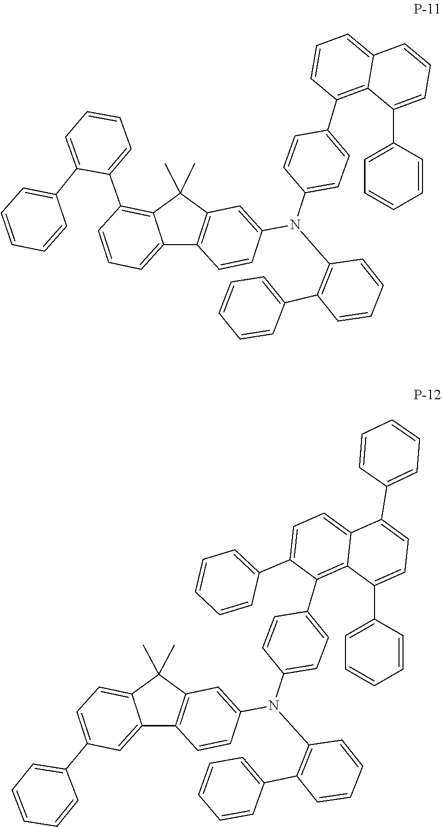 [SMILES: CC1(C)c2cc(N(c3ccc(-c4cccc5cccc(-c6ccccc6)c45)cc3)c3ccccc3-c3ccccc3)ccc2-c2cccc(-c3ccccc3-c3ccccc3)c21 ; CC1(C)c2ccc(-c3ccccc3)cc2-c2ccc(N(c3ccc(-c4c(-c5ccccc5)ccc5c(-c6ccccc6)ccc(-c6ccccc6)c45)cc3)c3ccccc3-c3ccccc3)cc21]

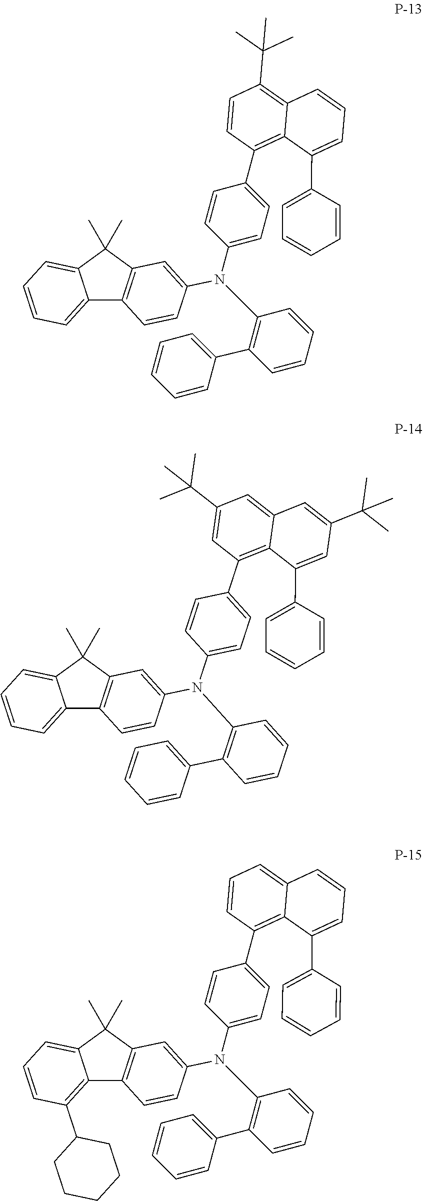 [SMILES: CC(C)(C)c1ccc(-c2ccc(N(c3ccc4c(c3)C(C)(C)c3ccccc3-4)c3ccccc3-c3ccccc3)cc2)c2c(-c3ccccc3)cccc12 ; CC(C)(C)c1cc(-c2ccccc2)c2c(-c3ccc(N(c4ccc5c(c4)C(C)(C)c4ccccc4-5)c4ccccc4-c4ccccc4)cc3)cc(C(C)(C)C)cc2c1 ; CC1(C)c2cc(N(c3ccc(-c4cccc5cccc(-c6ccccc6)c45)cc3)c3ccccc3-c3ccccc3)ccc2-c2c(C3CCCCC3)cccc21]

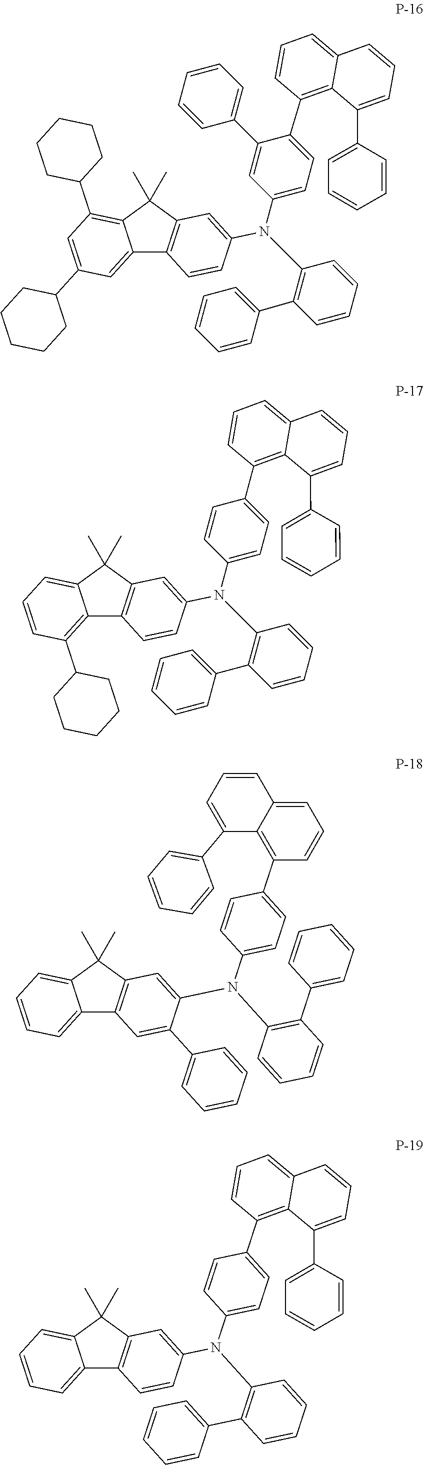 [SMILES: CC1(C)c2cc(N(c3ccc(-c4cccc5cccc(-c6ccccc6)c45)c(-c4ccccc4)c3)c3ccccc3-c3ccccc3)ccc2-c2cc(C3CCCCC3)cc(C3CCCCC3)c21 ; CC1(C)c2cc(N(c3ccc(-c4cccc5cccc(-c6ccccc6)c45)cc3)c3ccccc3-c3ccccc3)ccc2-c2c(C3CCCCC3)cccc21 ; CC1(C)c2ccccc2-c2cc(-c3ccccc3)c(N(c3ccc(-c4cccc5cccc(-c6ccccc6)c45)cc3)c3ccccc3-c3ccccc3)cc21 ; CC1(C)c2ccccc2-c2ccc(N(c3ccc(-c4cccc5cccc(-c6ccccc6)c45)cc3)c3ccccc3-c3ccccc3)cc21]

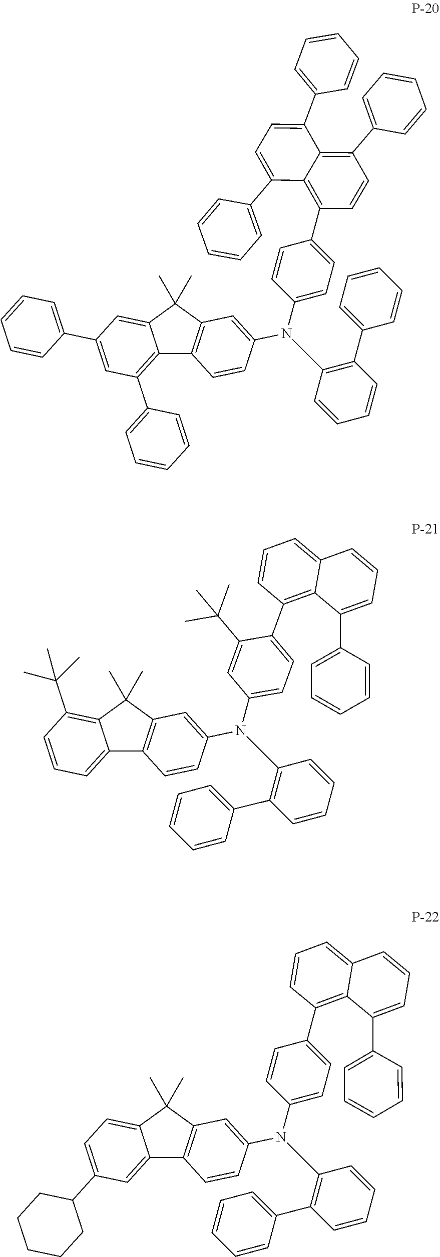 [SMILES: CC1(C)c2cc(N(c3ccc(-c4ccc(-c5ccccc5)c5c(-c6ccccc6)ccc(-c6ccccc6)c45)cc3)c3ccccc3-c3ccccc3)ccc2-c2c(-c3ccccc3)cc(-c3ccccc3)cc21 ; CC(C)(C)c1cc(N(c2ccc3c(c2)C(C)(C)c2c-3cccc2C(C)(C)C)c2ccccc2-c2ccccc2)ccc1-c1cccc2cccc(-c3ccccc3)c12 ; CC1(C)c2ccc(C3CCCCC3)cc2-c2ccc(N(c3ccc(-c4cccc5cccc(-c6ccccc6)c45)cc3)c3ccccc3-c3ccccc3)cc21]

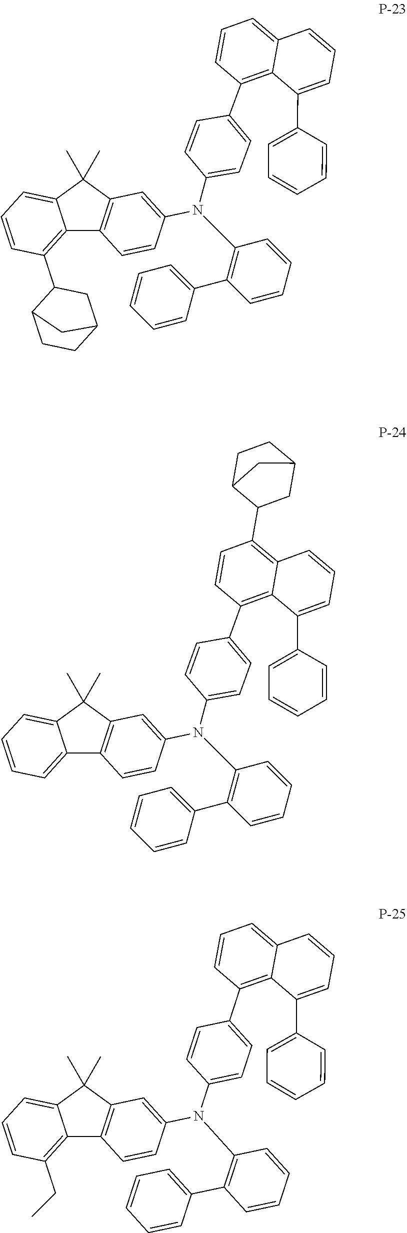 [SMILES: CC1(C)c2cc(N(c3ccc(-c4cccc5cccc(-c6ccccc6)c45)cc3)c3ccccc3-c3ccccc3)ccc2-c2c(C3CC4CCC3C4)cccc21 ; CC1(C)c2ccccc2-c2ccc(N(c3ccc(-c4ccc(C5CC6CCC5C6)c5cccc(-c6ccccc6)c45)cc3)c3ccccc3-c3ccccc3)cc21 ; CCc1cccc2c1-c1ccc(N(c3ccc(-c4cccc5cccc(-c6ccccc6)c45)cc3)c3ccccc3-c3ccccc3)cc1C2(C)C]

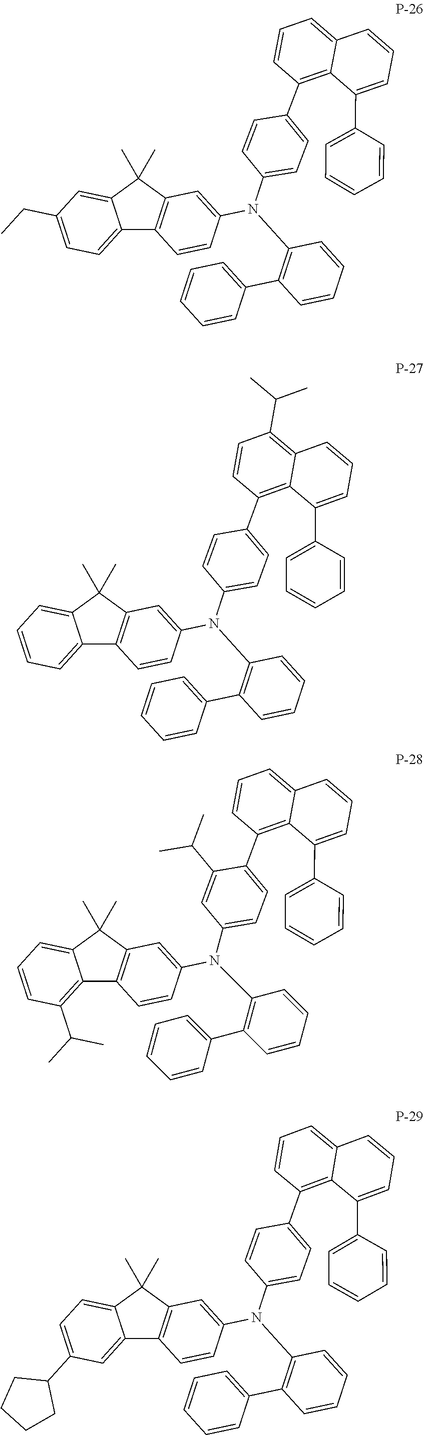 [SMILES: CCc1ccc2c(c1)C(C)(C)c1cc(N(c3ccc(-c4cccc5cccc(-c6ccccc6)c45)cc3)c3ccccc3-c3ccccc3)ccc1-2 ; CC(C)c1ccc(-c2ccc(N(c3ccc4c(c3)C(C)(C)c3ccccc3-4)c3ccccc3-c3ccccc3)cc2)c2c(-c3ccccc3)cccc12 ; CC(C)c1cc(N(c2ccc3c(c2)C(C)(C)c2cccc(C(C)C)c2-3)c2ccccc2-c2ccccc2)ccc1-c1cccc2cccc(-c3ccccc3)c12 ; CC1(C)c2ccc(C3CCCC3)cc2-c2ccc(N(c3ccc(-c4cccc5cccc(-c6ccccc6)c45)cc3)c3ccccc3-c3ccccc3)cc21]

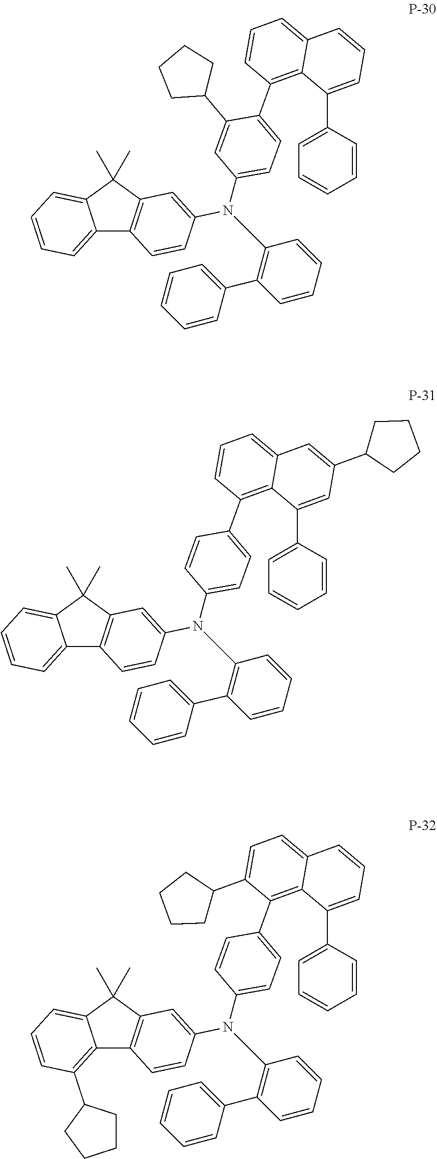 [SMILES: CC1(C)c2ccccc2-c2ccc(N(c3ccc(-c4cccc5cccc(-c6ccccc6)c45)c(C4CCCC4)c3)c3ccccc3-c3ccccc3)cc21 ; CC1(C)c2ccccc2-c2ccc(N(c3ccc(-c4cccc5cc(C6CCCC6)cc(-c6ccccc6)c45)cc3)c3ccccc3-c3ccccc3)cc21 ; CC1(C)c2cc(N(c3ccc(-c4c(C5CCCC5)ccc5cccc(-c6ccccc6)c45)cc3)c3ccccc3-c3ccccc3)ccc2-c2c(C3CCCC3)cccc21]

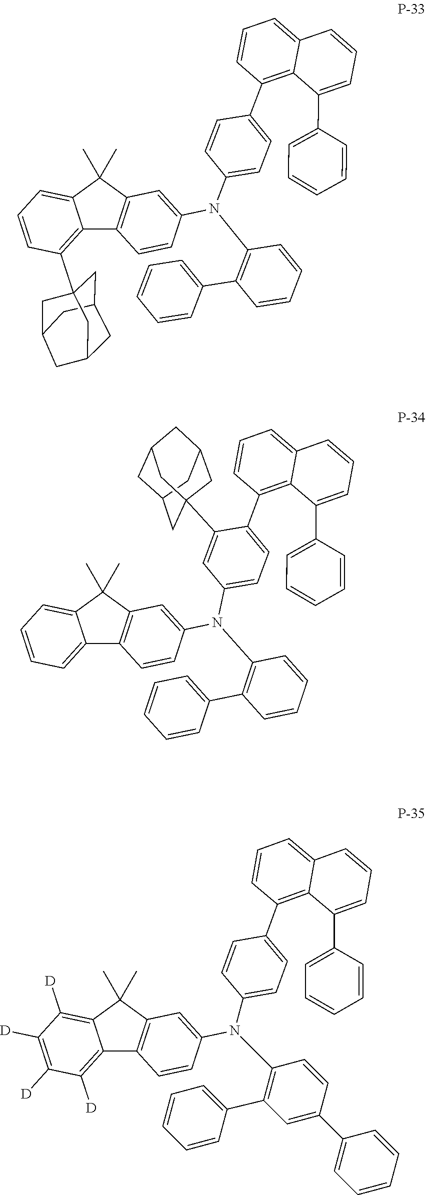 [SMILES: CC1(C)c2cc(N(c3ccc(-c4cccc5cccc(-c6ccccc6)c45)cc3)c3ccccc3-c3ccccc3)ccc2-c2c(C34CC5CC(CC(C5)C3)C4)cccc21 ; CC1(C)c2ccccc2-c2ccc(N(c3ccc(-c4cccc5cccc(-c6ccccc6)c45)c(C45CC6CC(CC(C6)C4)C5)c3)c3ccccc3-c3ccccc3)cc21 ; [2H]c1c([2H])c([2H])c2c(c1[2H])-c1ccc(N(c3ccc(-c4cccc5cccc(-c6ccccc6)c45)cc3)c3ccc(-c4ccccc4)cc3-c3ccccc3)cc1C2(C)C]

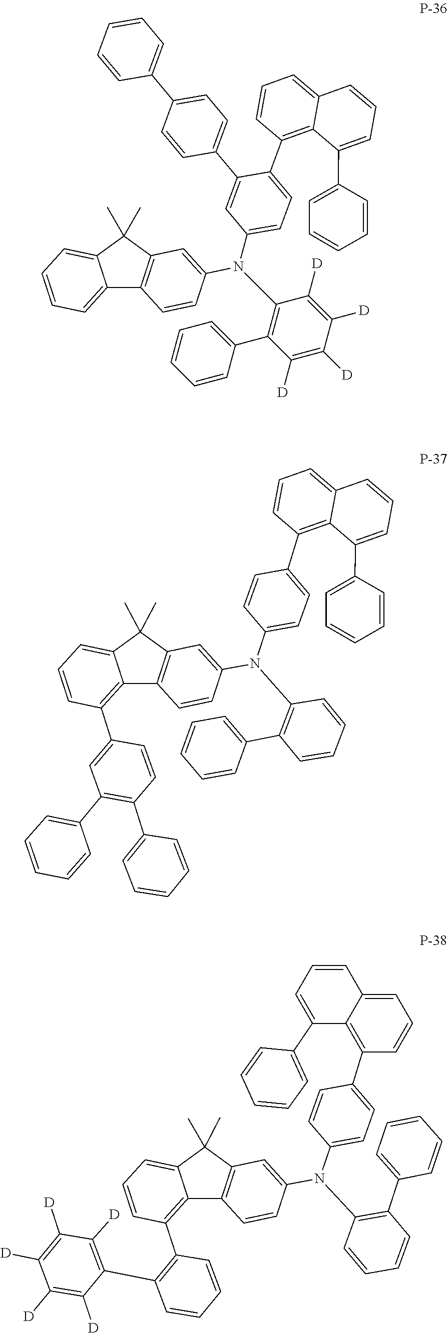 [SMILES: [2H]c1c([2H])c([2H])c(N(c2ccc(-c3cccc4cccc(-c5ccccc5)c34)c(-c3ccc(-c4ccccc4)cc3)c2)c2ccc3c(c2)C(C)(C)c2ccccc2-3)c(-c2ccccc2)c1[2H] ; CC1(C)c2cc(N(c3ccc(-c4cccc5cccc(-c6ccccc6)c45)cc3)c3ccccc3-c3ccccc3)ccc2-c2c(-c3ccc(-c4ccccc4)c(-c4ccccc4)c3)cccc21 ; [2H]c1c([2H])c([2H])c(-c2ccccc2-c2cccc3c2-c2ccc(N(c4ccc(-c5cccc6cccc(-c7ccccc7)c56)cc4)c4ccccc4-c4ccccc4)cc2C3(C)C)c([2H])c1[2H]]

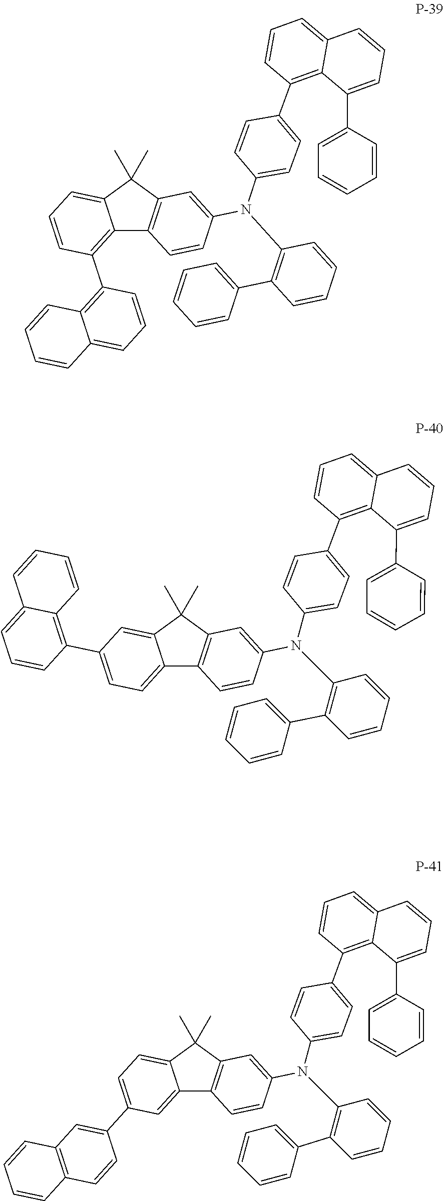 [SMILES: CC1(C)c2cc(N(c3ccc(-c4cccc5cccc(-c6ccccc6)c45)cc3)c3ccccc3-c3ccccc3)ccc2-c2c(-c3cccc4ccccc34)cccc21 ; CC1(C)c2cc(-c3cccc4ccccc34)ccc2-c2ccc(N(c3ccc(-c4cccc5cccc(-c6ccccc6)c45)cc3)c3ccccc3-c3ccccc3)cc21 ; CC1(C)c2ccc(-c3ccc4ccccc4c3)cc2-c2ccc(N(c3ccc(-c4cccc5cccc(-c6ccccc6)c45)cc3)c3ccccc3-c3ccccc3)cc21]

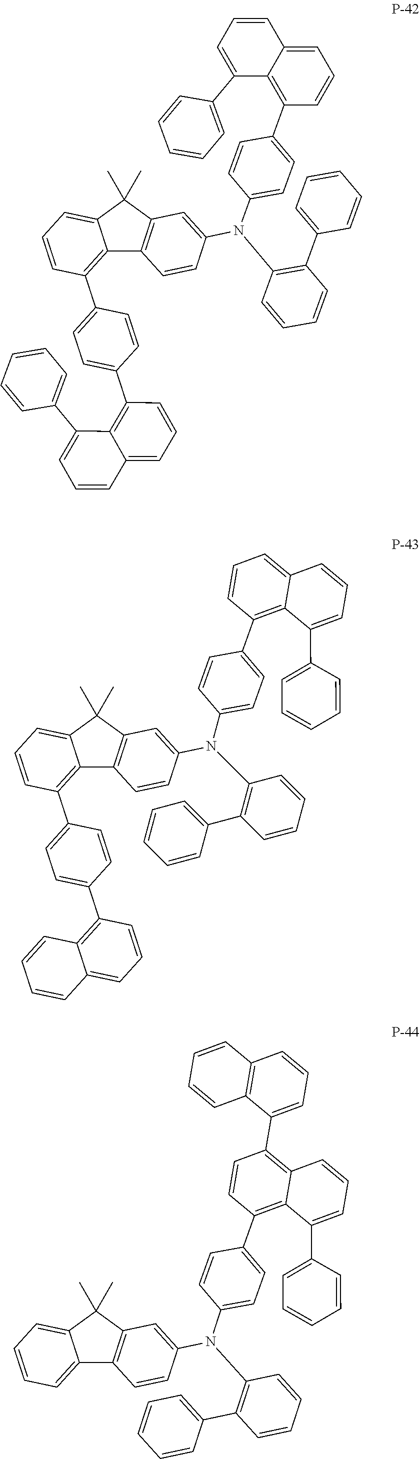 [SMILES: CC1(C)c2cc(N(c3ccc(-c4cccc5cccc(-c6ccccc6)c45)cc3)c3ccccc3-c3ccccc3)ccc2-c2c(-c3ccc(-c4cccc5cccc(-c6ccccc6)c45)cc3)cccc21 ; CC1(C)c2cc(N(c3ccc(-c4cccc5cccc(-c6ccccc6)c45)cc3)c3ccccc3-c3ccccc3)ccc2-c2c(-c3ccc(-c4cccc5ccccc45)cc3)cccc21 ; CC1(C)c2ccccc2-c2ccc(N(c3ccc(-c4ccc(-c5cccc6ccccc56)c5cccc(-c6ccccc6)c45)cc3)c3ccccc3-c3ccccc3)cc21]

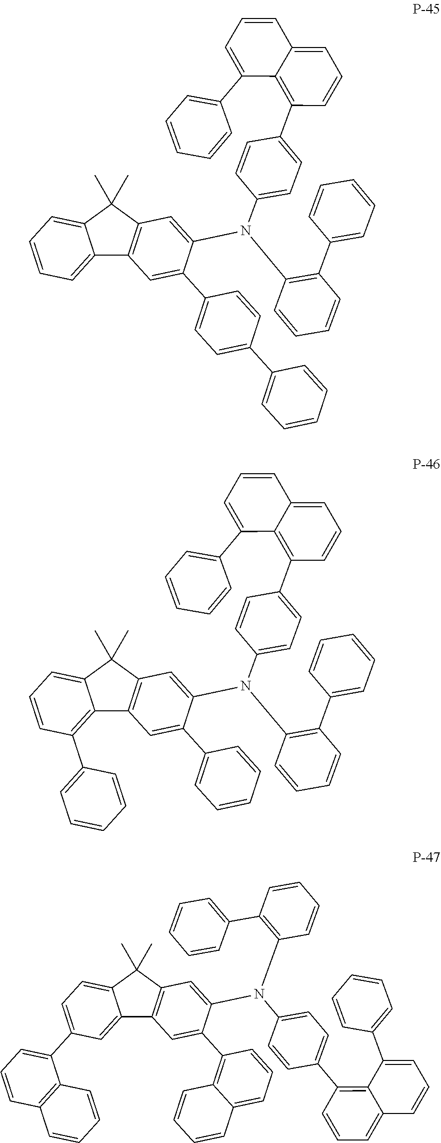

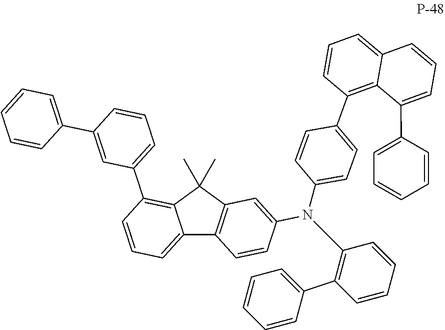 [SMILES: CC1(C)c2cc(N(c3ccc(-c4cccc5cccc(-c6ccccc6)c45)cc3)c3ccccc3-c3ccccc3)ccc2-c2cccc(-c3cccc(-c4ccccc4)c3)c21]

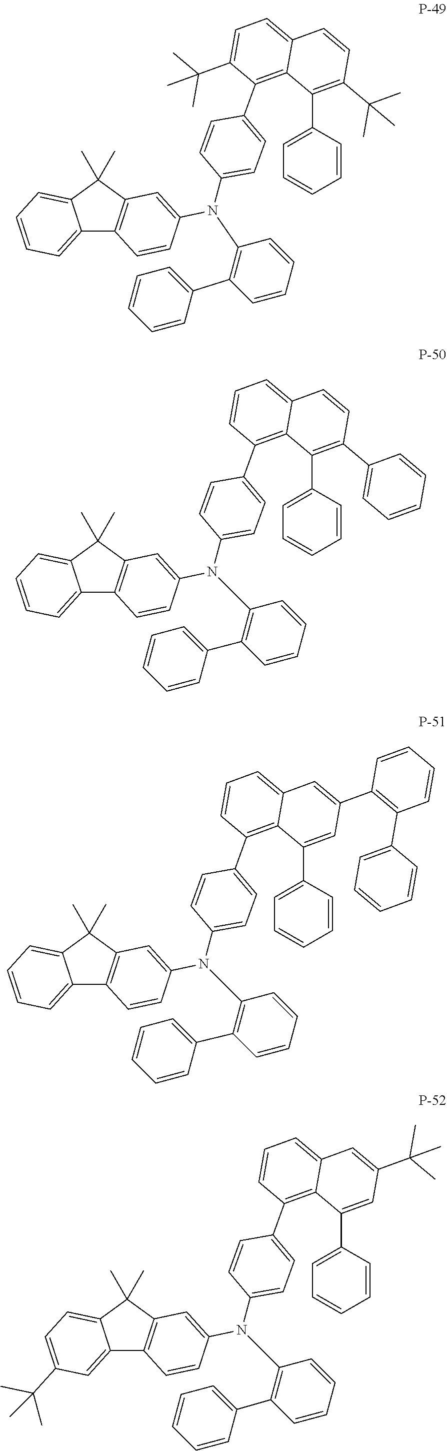

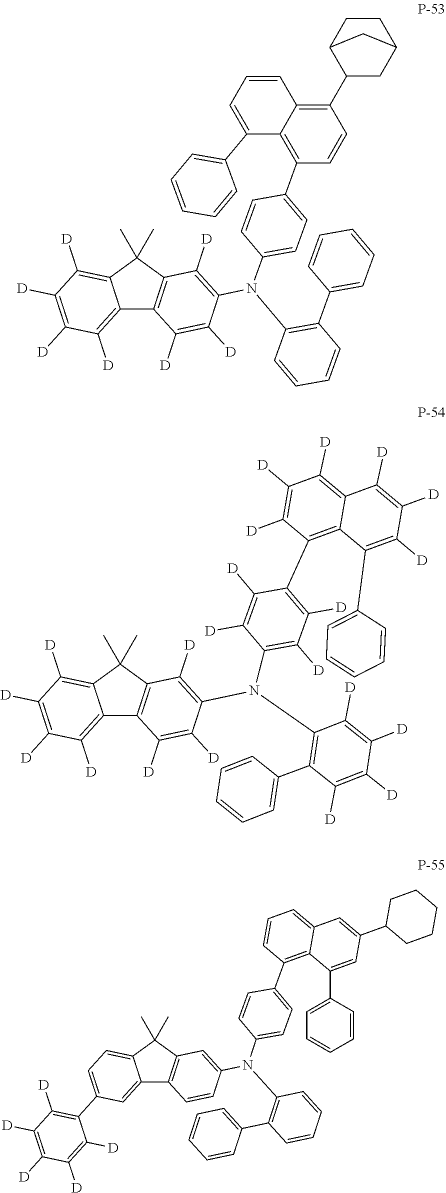 [SMILES: [2H]c1c([2H])c([2H])c2c(c1[2H])-c1c([2H])c([2H])c(N(c3ccc(-c4ccc(C5CC6CCC5C6)c5cccc(-c6ccccc6)c45)cc3)c3ccccc3-c3ccccc3)c([2H])c1C2(C)C ; [2H]c1c([2H])c([2H])c(N(c2c([2H])c([2H])c(-c3c([2H])c([2H])c([2H])c4c([2H])c([2H])c([2H])c(-c5ccccc5)c34)c([2H])c2[2H])c2c([2H])c([2H])c3c(c2[2H])C(C)(C)c2c([2H])c([2H])c([2H])c([2H])c2-3)c(-c2ccccc2)c1[2H] ; [2H]c1c([2H])c([2H])c(-c2ccc3c(c2)-c2ccc(N(c4ccc(-c5cccc6cc(C7CCCCC7)cc(-c7ccccc7)c56)cc4)c4ccccc4-c4ccccc4)cc2C3(C)C)c([2H])c1[2H]]

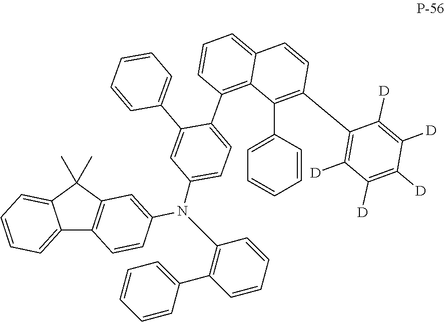 [SMILES: [2H]c1c([2H])c([2H])c(-c2ccc3cccc(-c4ccc(N(c5ccc6c(c5)C(C)(C)c5ccccc5-6)c5ccccc5-c5ccccc5)cc4-c4ccccc4)c3c2-c2ccccc2)c([2H])c1[2H]]

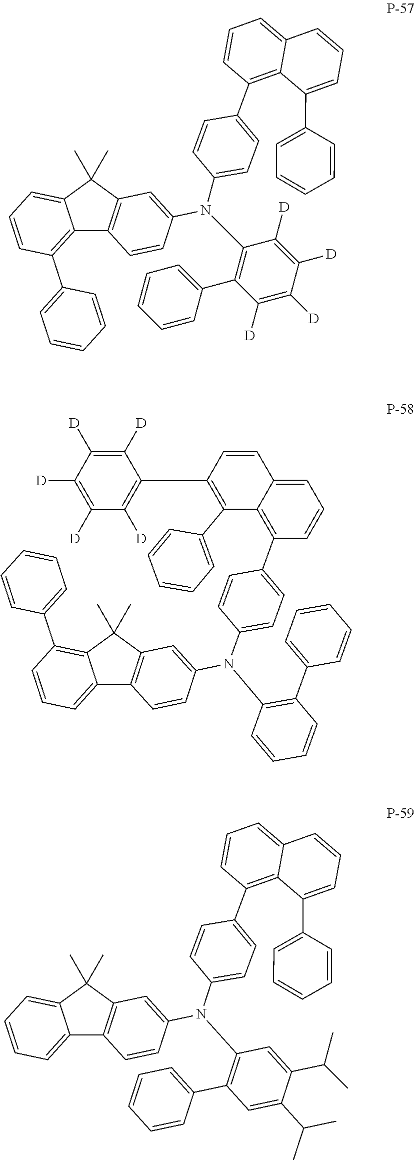 [SMILES: [2H]c1c([2H])c([2H])c(N(c2ccc(-c3cccc4cccc(-c5ccccc5)c34)cc2)c2ccc3c(c2)C(C)(C)c2cccc(-c4ccccc4)c2-3)c(-c2ccccc2)c1[2H] ; [2H]c1c([2H])c([2H])c(-c2ccc3cccc(-c4ccc(N(c5ccc6c(c5)C(C)(C)c5c(-c7ccccc7)cccc5-6)c5ccccc5-c5ccccc5)cc4)c3c2-c2ccccc2)c([2H])c1[2H] ; CC(C)c1cc(-c2ccccc2)c(N(c2ccc(-c3cccc4cccc(-c5ccccc5)c34)cc2)c2ccc3c(c2)C(C)(C)c2ccccc2-3)cc1C(C)C]

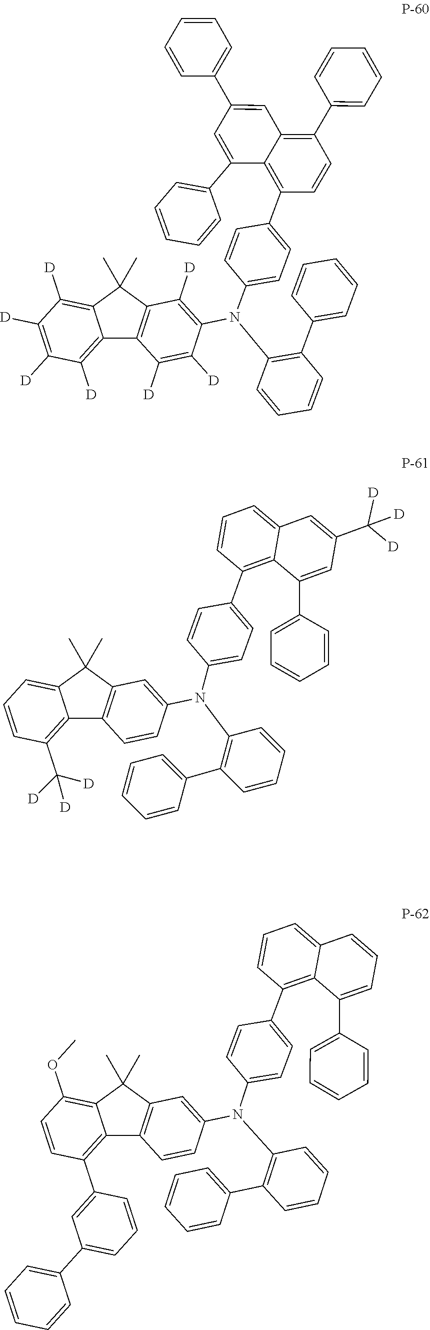 [SMILES: [2H]c1c([2H])c([2H])c2c(c1[2H])-c1c([2H])c([2H])c(N(c3ccc(-c4ccc(-c5ccccc5)c5cc(-c6ccccc6)cc(-c6ccccc6)c45)cc3)c3ccccc3-c3ccccc3)c([2H])c1C2(C)C ; [2H]C([2H])([2H])c1cc(-c2ccccc2)c2c(-c3ccc(N(c4ccc5c(c4)C(C)(C)c4cccc(C([2H])([2H])[2H])c4-5)c4ccccc4-c4ccccc4)cc3)cccc2c1 ; COc1ccc(-c2cccc(-c3ccccc3)c2)c2c1C(C)(C)c1cc(N(c3ccc(-c4cccc5cccc(-c6ccccc6)c45)cc3)c3ccccc3-c3ccccc3)ccc1-2]

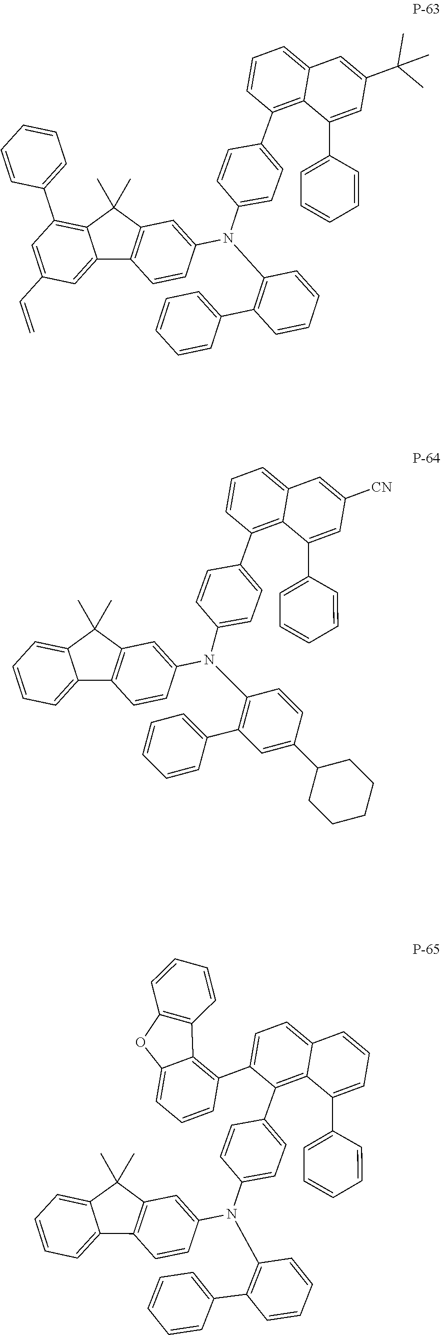 [SMILES: C=Cc1cc(-c2ccccc2)c2c(c1)-c1ccc(N(c3ccc(-c4cccc5cc(C(C)(C)C)cc(-c6ccccc6)c45)cc3)c3ccccc3-c3ccccc3)cc1C2(C)C ; CC1(C)c2ccccc2-c2ccc(N(c3ccc(-c4cccc5cc(C#N)cc(-c6ccccc6)c45)cc3)c3ccc(C4CCCCC4)cc3-c3ccccc3)cc21 ; CC1(C)c2ccccc2-c2ccc(N(c3ccc(-c4c(-c5cccc6oc7ccccc7c56)ccc5cccc(-c6ccccc6)c45)cc3)c3ccccc3-c3ccccc3)cc21]

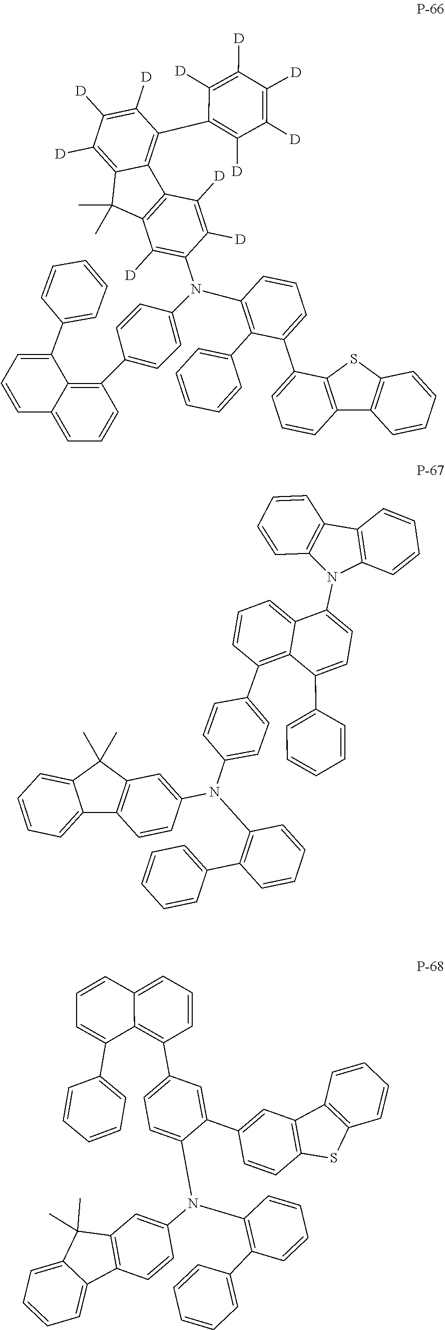 [SMILES: [2H]c1c([2H])c([2H])c(-c2c([2H])c([2H])c([2H])c3c2-c2c([2H])c([2H])c(N(c4ccc(-c5cccc6cccc(-c7ccccc7)c56)cc4)c4cccc(-c5cccc6c5sc5ccccc56)c4-c4ccccc4)c([2H])c2C3(C)C)c([2H])c1[2H] ; CC1(C)c2ccccc2-c2ccc(N(c3ccc(-c4cccc5c(-n6c7ccccc7c7ccccc76)ccc(-c6ccccc6)c45)cc3)c3ccccc3-c3ccccc3)cc21 ; CC1(C)c2ccccc2-c2ccc(N(c3ccccc3-c3ccccc3)c3ccc(-c4cccc5cccc(-c6ccccc6)c45)cc3-c3ccc4sc5ccccc5c4c3)cc21]

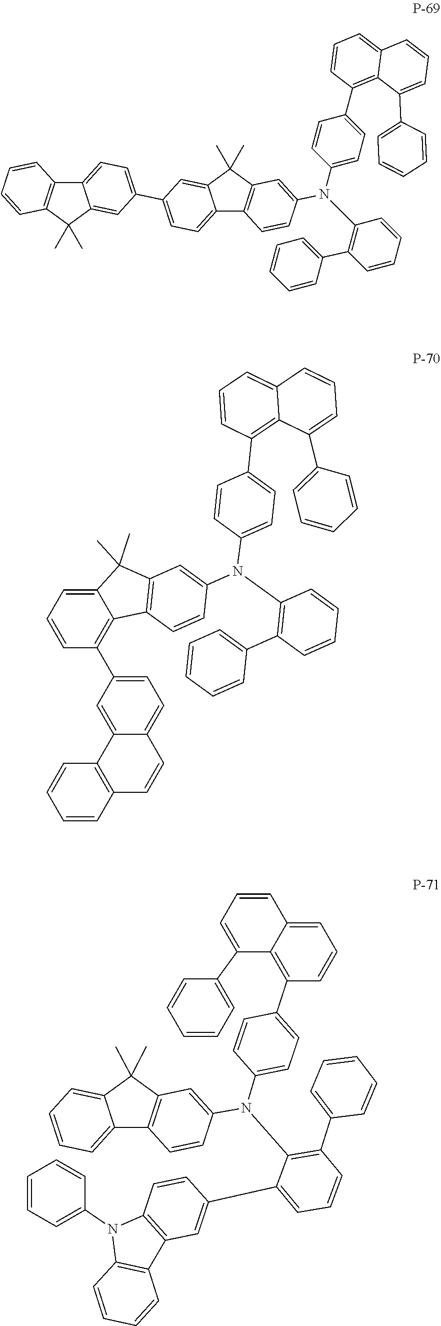 [SMILES: CC1(C)c2ccccc2-c2ccc(-c3ccc4c(c3)C(C)(C)c3cc(N(c5ccc(-c6cccc7cccc(-c8ccccc8)c67)cc5)c5ccccc5-c5ccccc5)ccc3-4)cc21 ; CC1(C)c2cc(N(c3ccc(-c4cccc5cccc(-c6ccccc6)c45)cc3)c3ccccc3-c3ccccc3)ccc2-c2c(-c3ccc4ccc5ccccc5c4c3)cccc21 ; CC1(C)c2ccccc2-c2ccc(N(c3ccc(-c4cccc5cccc(-c6ccccc6)c45)cc3)c3c(-c4ccccc4)cccc3-c3ccc4c(c3)c3ccccc3n4-c3ccccc3)cc21]

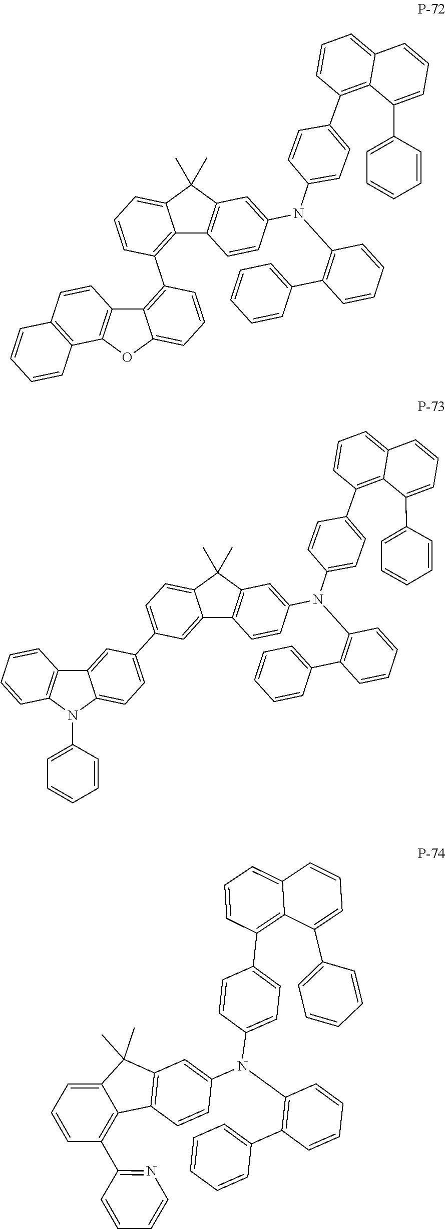 [SMILES: CC1(C)c2cc(N(c3ccc(-c4cccc5cccc(-c6ccccc6)c45)cc3)c3ccccc3-c3ccccc3)ccc2-c2c(-c3cccc4oc5c6ccccc6ccc5c34)cccc21 ; CC1(C)c2ccc(-c3ccc4c(c3)c3ccccc3n4-c3ccccc3)cc2-c2ccc(N(c3ccc(-c4cccc5cccc(-c6ccccc6)c45)cc3)c3ccccc3-c3ccccc3)cc21 ; CC1(C)c2cc(N(c3ccc(-c4cccc5cccc(-c6ccccc6)c45)cc3)c3ccccc3-c3ccccc3)ccc2-c2c(-c3ccccn3)cccc21]

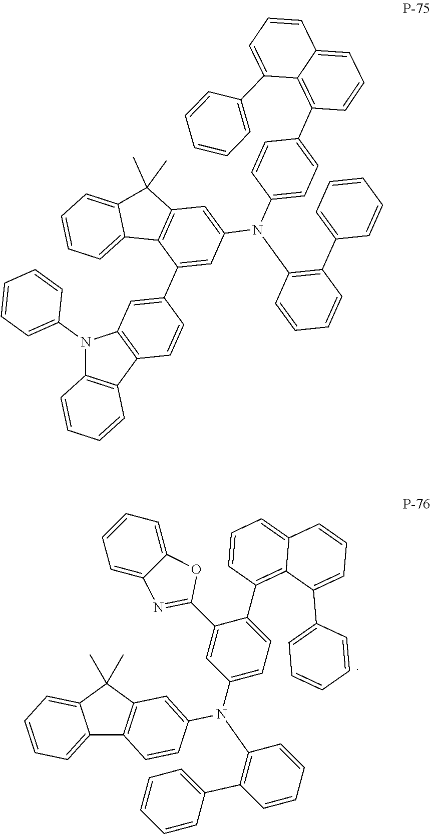 [SMILES: CC1(C)c2ccccc2-c2c(-c3ccc4c5ccccc5n(-c5ccccc5)c4c3)cc(N(c3ccc(-c4cccc5cccc(-c6ccccc6)c45)cc3)c3ccccc3-c3ccccc3)cc21 ; CC1(C)c2ccccc2-c2ccc(N(c3ccc(-c4cccc5cccc(-c6ccccc6)c45)c(-c4nc5ccccc5o4)c3)c3ccccc3-c3ccccc3)cc21]

7. An organic electronic element comprising an anode, a cathode, and an organic material layer formed between the anode and the cathode, wherein the organic material layer comprises a single compound or 2 or more compounds represented by Formula 1 of claim 1.

8. The organic electronic element of claim 7, wherein the organic material layer comprises any one of a hole injection layer, a hole transport layer, an emitting-auxiliary layer, an emitting layer, an electron transport auxiliary layer, an electron transport layer, and an electron injection layer.

9. The organic electronic element of claim 7, wherein the organic material layer is a hole transport layer.

10. The organic electronic element of claim 7, further comprising a light efficiency enhancing layer formed on at least one surface of the anode and the cathode, the surface being opposite to the organic material layer.

11. The organic electronic element according to claim 7, wherein the organic material layer comprises 2 or more stacks comprising a hole transport layer, an emitting layer and an electron transport layer sequentially formed on the anode.

12. The organic electronic element according to claim 11, the organic material layer further comprises a charge generation layer formed between the 2 or more stacks.

13. An electronic device comprising a display device comprising the organic electronic element of claim 7; and a control unit for driving the display device.

14. An electronic device according to claim 13, wherein the organic electronic element is at least one of an OLED, an organic solar cell, an organic photo conductor (OPC), organic transistor (organic TFT) and an element for monochromic or white illumination.

15. A method for reusing a compound represented by Formula 1 of claim 1 comprising:
recovering a crude organic light emitting material comprising the compound of Formula 1 from a deposition apparatus used in a process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.
